SEQUENCE LISTING (50 sequences)

SEQ ID NO: 1 - RNA, 23 nt, Homo Sapiens
ugugcaaaucuaugcaaaacuga

SEQ ID NO: 2 - RNA, 23 nt, Homo Sapiens
ugugcaaauccaugcaaaacuga

SEQ ID NO: 3 - RNA, 22 nt, Homo Sapiens
uagcaccaucugaaaucgguua

SEQ ID NO: 4 - RNA, 23 nt, Homo Sapiens
uguaaacauccuacacucucagc

SEQ ID NO: 5 - RNA, 22 nt, Homo Sapiens
uggcagugucuuagcugguugu

SEQ ID NO: 6 - RNA, 22 nt, Homo Sapiens
uuaaggcacgcggugaaugcca

SEQ ID NO: 7 - RNA, 22 nt, Homo Sapiens
uuugguccccuucaaccagcug

SEQ ID NO: 8 - RNA, 22 nt, Homo Sapiens
uuugguccccuucaaccagcua

SEQ ID NO: 9 - RNA, 21 nt, Homo Sapiens
cuagacugaagcuccuugagg

SEQ ID NO: 10 - RNA, 23 nt, Homo Sapiens
caacggaaucccaaaagcagcug

SEQ ID NO: 11 - RNA, 21 nt, Homo Sapiens
gccccugggccuauccuagaa

SEQ ID NO: 12 - RNA, 23 nt, Homo Sapiens
ggcagguucucacccucucuagg

SEQ ID NO: 13 - RNA, 23 nt, Homo Sapiens
uauucauuuauccccagccuaca

SEQ ID NO: 14 - RNA, 21 nt, Homo Sapiens
ccuggaaacacugagguugug

SEQ ID NO: 15 - RNA, 21 nt, Homo Sapiens
uagcagcacagaaauauuggc

SEQ ID NO: 16 - RNA, 22 nt, Homo Sapiens
auauaauacaaccugcuaagug

SEQ ID NO: 17 - RNA, 22 nt, Homo Sapiens
gagcuuauucauaaaagugcag

SEQ ID NO: 18 - RNA, 21 nt, Homo Sapiens
uucaaguaauucaggauaggu

SEQ ID NO: 19 - RNA, 22 nt, Homo Sapiens
uaaugccccuaaaaauccuuau

SEQ ID NO: 20 - RNA, 22 nt, Homo Sapiens
uagcaccauuugaaaucgguua

SEQ ID NO: 21 - RNA, 22 nt, Homo Sapiens
aacccguagauccgaucuugug

SEQ ID NO: 22 - RNA, 22 nt, Homo Sapiens
cuccugacuccagguccugugu

SEQ ID NO: 23 - RNA, 23 nt, Homo Sapiens
uagugcaauauugcuuauagggu

SEQ ID NO: 24 - RNA, 82 nt, Homo Sapiens
gcaguccucuguuaguuuugcauaguugcacuacaagaagaauguaguugugcaaaucuaugcaaaacugaugguggccugc

SEQ ID NO: 25 - RNA, 87 nt, Homo Sapiens
cacuguucuaugguuaguuuugcagguuugcauccagcugugugauauucugcugugcaaauccaugcaaaacugacugugguagug

SEQ ID NO: 26 - RNA, 96 nt, Homo Sapiens
acauugcuacuuacaauuaguuuugcagguuugcauuucagcguauauauguauauguggcugugcaaauccaugcaaaacugauugugauaaugu

SEQ ID NO: 27 - RNA, 64 nt, Homo Sapiens
augacugauuucuuuugguguucagagucaauauaauuuucuagcaccaucugaaaucgguuau

SEQ ID NO: 28 - RNA, 89 nt, Homo Sapiens
accaugcuguaguguguguaaacauccuacacucucagcugugagcucaagguggcugggagaggguuguuuacuccuucugccaugga

SEQ ID NO: 29 - RNA, 72 nt, Homo Sapiens
agauacuguaaacauccuacacucucagcuguggaaaguaagaaagcugggagaaggcuguuuacucuuucu

SEQ ID NO: 30 - RNA, 110 nt, Homo Sapiens
ggccagcugugaguguuucuuuggcagugucuuagcugguuguugugagcaauaguaaggaagcaaucagcaaguauacugcccuagaagugcugcacguuguggggccc

SEQ ID NO: 31 - RNA, 107 nt, Homo Sapiens
aucaagaucagaggcucugcccuccguguucacagcggaccuugauuuaaugucauacaauuaaggcacgcggugaaugccaagagcggagccuacggcugcacuug

SEQ ID NO: 32 - RNA, 88 nt, Homo Sapiens
acaaugcuuugcuagagcugguaaaauggaaccaaaucgccucuucaauggauuugguccccuucaaccagcuguagcuaugcauuga

SEQ ID NO: 33 - RNA, 102 nt, Homo Sapiens
gggagccaaaugcuuugcuagagcugguaaaauggaaccaaaucgacuguccaauggauuugguccccuucaaccagcuguagcugugcauugauggcgccg

SEQ ID NO: 34 - RNA, 119 nt, Homo Sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 35 - RNA, 90 nt, Homo Sapiens
uuuccugcccucgaggagcucacagucuaguaugucucauccccuacuagacugaagcuccuugaggacagggauggucauacucaccuc

SEQ ID NO: 36 - RNA, 92 nt, Homo Sapiens
cggcuggacagcgggcaacggaaucccaaaagcagcuguugucuccagagcauuccagcugcgcuuggauuucguccccugcucuccugccu

SEQ ID NO: 37 - RNA, 94 nt, Homo Sapiens
gaguuugguuuuguuuggguuuguucuagguauggucccagggaucccagaucaaaccaggccccugggccuauccuagaaccaaccuaagcuc

SEQ ID NO: 38 - RNA, 98 nt, Homo Sapiens
guguaguagagcuaggaggagaggguccuggagaagcguggaccgguccggguggguuccggcagguucucacccucucuaggccccauucuccucug

SEQ ID NO: 39 - RNA, 82 nt, Homo Sapiens
gaacauugaaacuggcuagggaaaaugauuggauagaaacuauuauucuauucauuuauccccagccuacaaaaugaaaaaa

SEQ ID NO: 40 - RNA, 76 nt, Homo Sapiens
uuagugguacuauaccucaguuuuaucagguguucuuaaaaucaccuggaaacacugagguugugucucacugaac

SEQ ID NO: 41 - RNA, 87 nt, Homo Sapiens
agcuucccuggcucuagcagcacagaaauauuggcacagggaagcgagucugccaauauuggcugugcugcuccaggcaggguggug

SEQ ID NO: 42 - RNA, 72 nt, Homo Sapiens
acucggauggauauaauacaaccugcuaaguguccuagcacuuagcagguuguauuaucauuguccgugucu

SEQ ID NO: 43 - RNA, 97 nt, Homo Sapiens
uagccagucagaaaugagcuuauucauaaaagugcaguauggugaagucaaucuguaauuuuauguauaagcuagucucugauugaaacaugcagca

SEQ ID NO: 44 - RNA, 77 nt, Homo Sapiens
ccgggacccaguucaaguaauucaggauagguugugugcuguccagccuguucuccauuacuuggcucggggaccgg

SEQ ID NO: 45 - RNA, 87 nt, Homo Sapiens
accgcagggaaaaugagggacuuuugggggcagauguguuuccauuccacuaucauaaugccccuaaaaauccuuauugcucuugca

SEQ ID NO: 46 - RNA, 111 nt, Homo Sapiens
agaguguucaaggacagcaagaaaaaugagggacuuucaggggcagcuguguuuucugacucagucauaaugccccuaaaaauccuuauuguucuugcagugugcaucggg

SEQ ID NO: 47 - RNA, 88 nt, Homo Sapiens
aucucuuacacaggcugaccgauuucuccugguguucagagucuguuuuugucuagcaccauuugaaaucgguuaugauguaggggga

SEQ ID NO: 48 - RNA, 81 nt, Homo Sapiens
cccauuggcauaaacccguagauccgaucuuguggugaaguggaccgcacaagcucgcuucuaugggucugugucagugug

SEQ ID NO: 49 - RNA, 66 nt, Homo Sapiens
agggcuccugacuccagguccuguguguuaccuagaaauagcacuggacuuggagucagaaggccu

SEQ ID NO: 50 - RNA, 115 nt, Homo Sapiens
ucuguuuaucaccagauccuagaacccuaucaauauugucucugcuguguaaauaguucugaguagugcaauauugcuuauaggguuuugguguuuggaaagaacaaugggcagg

(19) United States
(12) Patent Application Publication
(10) Pub. No.: US 2021/0147944 A1
(43) Pub. Date: May 20, 2021

(54) METHODS FOR MONITORING AND TREATING PROSTATE CANCER

(71) Applicants: UNITY HEALTH TORONTO, Toronto (CA); UNIVERSITY HEALTH NETWORK, Toronto (CA)

(72) Inventors: George M. Yousef, North York (CA); Neil Eric Fleshner, Toronto (CA)

(21) Appl. No.: 17/046,454

(22) PCT Filed: Apr. 10, 2019
(86) PCT No.: PCT/CA2019/050437

§ 371 Date: Oct. 9, 2020

Related U.S. Application Data
(60) Provisional application No. 62/655,443, filed on Apr. 10, 2018.

(51) Int. Cl. C12Q 1/6886; G16B 40/00; G16B 25/10

(52) U.S. Cl. CPC C12Q 1/6886; G16B 40/00; G16B 25/10; C12Q 2600/112; C12Q 2600/158; C12Q 2600/118; C12Q 2600/106; C12Q 2600/178

(57) ABSTRACT

The present disclosure provides methods for monitoring prostate cancer by determining or measuring exosomal miRNA levels in a biological sample to provide a biological sample exosomal miRNA profile comprising exosomal miRNA levels and determining or measuring the level of similarity of said biological sample exosomal miRNA profile to one or more control profiles. The present disclosure also provides kits for monitoring prostate cancer progression and methods for selecting a treatment for prostate cancer.

### RELATED APPLICATION

This application claims priority to U.S. Provisional Patent Application No. 62/655,443 filed on Apr. 10, 2018, the content of which is hereby incorporated by reference in its entirety.

### FIELD

The disclosure relates to methods and kits for monitoring prostate cancer in a human subject. The disclosure provides methods that distinguish between clinically aggressive prostate tumors from tumors that are clinically indolent by measuring levels of exosomal miRNA expression from blood or urine.

### BACKGROUND

Prostate cancer is the most common cancer in adult men worldwide^{2, 18}. The introduction of prostate specific antigen (PSA) testing a couple of decades ago has led to an unprecedented increase in the diagnosis of prostate cancer¹⁴. However, this was associated with the problem of overdiagnosis and over-treatment^{7, 12}. Many of the PSA-identified cancers are discovered in an early phase and are clinically considered as indolent: cancers that will never lead to mortality. A number of studies have shown that patients with indolent (i.e. clinically non-aggressive) prostate cancers are more likely to die from other causes rather than prostate cancer in their life time. Moreover, PSA lacks sensitivity and specificity for the diagnosis of prostate cancer leading to false positive results. Although PSA has been used to assist the estimation of disease aggressiveness, it lacks accuracy in this regard and preoperative PSA levels are not enough to predict tumor prognosis or guide treatment decisions.

More recently, a new trend of conserve management, also known as active surveillance, has been adopted in Canada, the USA and other countries¹². In this approach, clinical assessment is done and patients with predicted clinically indolent disease (i.e. low Gleason and low volume cancer) are given the choice of active surveillance which includes annual biopsy and PSA measurement plus monitoring by imaging. The criteria for active surveillance are, however, not uniform^{4, 13, 19}. Several groups have set different criteria for active surveillance, but none can be considered as a gold standard for this purpose. Most criteria rely on PSA or Gleason grade of the biopsy specimen, either of which suffers from drawbacks. PSA measurement might not be the most accurate. Biopsy specimen is not always representative of the entire lesion that can only be assessed in prostatectomy. This results in inaccuracy in the treatment decision and in certain cases being misassigned to active surveillance only then switched to radical prostatectomy afterwards.

### SUMMARY

The present disclosure provides methods that distinguish between clinically aggressive, i.e. tumors with high chance of relapse and those tumors of high Gleason grade and high tumor volume, and tumors that are clinically indolent, by measuring levels of exosomal miRNA expression from blood or urine in preoperative patients. In this regard, the methods of the present disclosure enable liquid biopsy of tumors which is, without wishing to be bound by theory, predicted to be fully representative of the entire lesion as opposed to the small biopsy specimen that can introduce diagnostic bias. miRNAs are stable molecules and readily detectable. Moreover, methods described herein were developed based on analysis of exosomal miRNAs which have been demonstrated to be more reproducible than freely circulating miRNAs.

Thus, the present disclosure provides a tool for objective assessment of monitoring prostate cancer progression, providing prognosis pre-operatively and accurately assessing patient outcome that can guide treatment decision making. The present inventors describe a novel method for monitoring and treating prostate cancer. As set out in the Examples, the present inventors have determined that it is possible to select a prostate cancer patient pre-operative of prostatectomy with known PSA level for active surveillance or prostatectomy by determining or measuring exosomal miRNA levels in a biological sample to provide a biological sample exosomal miRNA profile and determining or measuring the level of similarity of said biological sample exosomal miRNA profile to one or more control profiles.

Accordingly, the present disclosure provides methods for monitoring prostate cancer progression in a patient preoperative of prostatectomy with a known age and/or PSA level, comprising:
(a) (i) determining or measuring exosomal miRNA levels in a biological sample to provide a biological sample exosomal miRNA profile comprising exosomal miRNA level of at least one, at least two, or at least three exosomal miRNA from miR-19a, miR-19b, miR-29a, miR-30c, miR-34a, miR-124a, miR-133a, miR-133b, miR-151-3p, miR-191, miR-331, miR-657, miR-664a and miR-875-3p, or (ii) determining or measuring exosomal miRNA levels in a biological sample to provide a biological sample exosomal miRNA profile comprising exosomal miRNA level of at least three exosomal miRNA from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a prostatectomy control profile; and/or (iii) a higher level of similarity to an active surveillance control profile than to a prostatectomy control profile indicates low disease aggressiveness; or wherein (iv) a low level of similarity of the sample exosomal miRNA profile to an active surveillance control profile; (v) a high level of similarity to a prostatectomy control profile; and/or (vi) a lower level of similarity to an active surveillance control profile than to a prostatectomy control profile indicates high disease aggressiveness.

In an embodiment, the biological sample exosomal miRNA profile and the one or more control profiles comprise:
(a) the levels of at least two serum exosomal miRNAs selected from miR-29a, miR-30c, miR-151-3p, miR-191 and miR-664a, or at least two urine exosomal miRNAs selected from miR-19a, miR-195, miR-331, miR-374-5p and miR-590-5p;
(b) the levels of at least three serum exosomal miRNAs selected from miR-19a, miR-29a, miR-151-3p and miR-664a; or at least three urine exosomal miRNAs selected from miR-19a, miR-26b, miR-331, miR-374-5p and miR-590-5p; and/or
(c) the levels of at least two serum exosomal miRNAs selected from miR-29a, miR-133a, miR-151-3p and miR-657; or at least one serum exosomal miRNA selected from miR-19a, miR-133a, miR-151-3p and miR-657 and at least one urine exosomal miRNA selected from miR-19a, miR-26a, miR-331 and miR-590.

In another embodiment, the methods for monitoring prostate cancer progression in a patient preoperative of prostatectomy with a known PSA level comprises the steps:
(a) providing at least one biological sample from a patient, wherein the biological sample is serum or urine;
(b) determining or measuring exosomal miRNA levels in a biological sample to provide a biological sample exosomal miRNA profile comprising exosomal miRNA level;
(c) determining or measuring the level of similarity of said biological sample exosomal miRNA profile to one or more control profiles, wherein (i) a high level of similarity of the sample profile to an active surveillance control profile; (ii) a low level of similarity to a prostatectomy control profile; and/or (iii) a higher level of similarity to an active surveillance control profile than to a prostatectomy control profile indicates low disease aggressiveness; or wherein (iv) a low level of similarity of the sample profile to an active surveillance control profile; (v) a high level of similarity to a prostatectomy control profile; and/or (vi) a lower level of similarity to an active surveillance control profile than to a prostatectomy control profile indicates high disease aggressiveness;
(d) selecting said patient for active surveillance when said patient has low disease aggressiveness, or selecting said patient for prostatectomy when said patient has high disease aggressiveness; and
(e) repeating (a)-(d) in about one year when said patient is selected for active surveillance.

In another embodiment, the determining or measuring of exosomal miRNA levels comprises using droplet digital PCR, digital molecular barcoding, or next-generation sequencing.

In another embodiment, the miRNA is measured as miRNA copies/mL sample.

In another embodiment, a numerical score based on biological sample exosomal miRNA profile is calculated according to the following formula:

P=1/[1+exp.(−xβ)]=exp.(xβ)/[1+exp.(xβ)]
where xβ is standard linear form in multivariable logistic regression analysis,
wherein the numerical score is used in Receiver Operating

Characteristic (ROC) analysis.

The present disclosure also provides a method for treating prostate cancer in a patient preoperative of prostatectomy with known age and/or PSA level, comprising:
(A) diagnosing said patient as a candidate for prostatectomy, comprising
   (a) providing at least one biological sample from a patient, wherein the biological sample is serum or urine;
   (b) (i) measuring said biological sample exosomal miRNA levels comprising exosomal miRNA level of at least one, at least two, or at least three exosomal miRNA from miR-19a, miR-19b, miR-29a, miR-30c, miR-34a, miR-124a, miR-133a, miR-133b, miR-151-3p, miR-191, miR-331, miR-657, miR-664a and miR-875-3p, or (ii) measuring said biological sample exosomal miRNA levels comprising exosomal miRNA level of at least three exosomal miRNA from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p;
   (c) determining said biological sample exosomal miRNA profile comprising miRNA levels of (b); and
   (d) determining or measuring the level of similarity of said biological sample exosomal miRNA profile to one or more control profiles, wherein (i) a low level of similarity of the sample profile to an active surveillance control profile; (ii) a high level of similarity to a prostatectomy control profile; and/or (iii) a lower level of similarity to an active surveillance control profile than to a prostatectomy control profile indicates high disease aggressiveness; or wherein (iv) a high level of similarity of the sample exosomal miRNA profile to an active surveillance control profile; (v) a low level of similarity to a prostatectomy control profile; and/or (vi) a higher level of similarity to an active surveillance control profile than to a prostatectomy control profile indicates low disease aggressiveness; and
(B) removing the prostate when said patient has high disease aggressiveness, or
   monitoring prostate cancer progression annually when said patient has low disease aggressiveness.

The present disclosure also provides a method for selecting a treatment for prostate cancer in a patient preoperative of prostatectomy with known PSA level, comprising:
(a) providing at least one biological sample from a patient, wherein the biological sample is serum or urine;
(b) measuring exosomal miRNA levels of said biological sample;
(c) determining exosomal miRNA profile of said biological sample;
(d) determining or measuring the level of similarity of said biological sample exosomal miRNA profile to one or more control profiles, wherein (i) a low level of similarity of the sample profile to an active surveillance control profile; (ii) a high level of similarity to a prostatectomy control profile; and/or (iii) a lower level of similarity to an active surveillance control profile than to a prostatectomy control profile indicates high disease aggressiveness; or wherein (iv) a high level of similarity of the sample exosomal miRNA profile to an active surveillance control profile; (v) a low level of similarity to a prostatectomy control profile; and/or (vi) a higher level of similarity to an active surveillance control profile than to a prostatectomy control profile indicates low disease aggressiveness; and
(e) selecting said patient for prostatectomy when said patient has high disease aggressiveness; or selecting said patient for monitoring prostate cancer progression annually when said patient has low disease aggressiveness.

The present disclosure further provides a kit for analyzing serum or urine sample to monitor prostate cancer progression in a patient comprising:
a probe that detects the presence of exosomal miRNA described herein; and
instructions for use,
wherein the patient is preoperative of prostatectomy with a known PSA level.

In an embodiment, the probe is a set of exosomal miRNA-specific primers.

In another embodiment, the set of exosomal miRNA-specific primers comprises primers targeting miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a or miR-875-3p.

In another embodiment, the set of exosomal miRNA-specific primers comprises primers targeting the exosomal miRNAs described herein.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments are described below in relation to the drawings in which:

FIG. 1 shows a graphical representation of cumulative and progression-free survival (measured in days) based on PSA (FIG. 1A) and Gleason score (FIGS. 1B, C, and D). (A) Using a cut-off of 11 ng/ml preoperative serum PSA was not able to distinguish between patients with biochemical failure from those who did not experience biochemical failure after prostatectomy. (B) The final Gleason score from prostatectomy was able to distinguish the patient cohort into distinct subgroups in terms of possibility of disease progression survival (biochemical failure). (C) Risk of recurrence was determined in Gleason 1+2 compared with Gleason 3+4+5. (D) Relative risk of recurrence was further determined within combined Gleason 3+4+5 subgroups with Gleason 1+2 high risk subgroups separated from Gleason 1+2 low risk subgroups.

FIG. 2 shows a graphical representation of cumulative and progression-free survival (measured in days) relative to: (A) miR-151-3p, (B) miR-664a, (C) miR-29a, (D) miR-191, or (E) miR-30c expression levels measured in preoperative serum.

FIG. 3 shows a graphical representation of cumulative and progression-free survival (measured in days) relative to (A) miR-29a, (B) miR-664a, or (C) miR-151-3p measured in urinary exosomes. Serum exosomal miRNAs had the ability to distinguish patients in Gleason Grade Group 1 into indolent and aggressive subgroups.

FIG. 4 shows a graphical representation of cumulative and progression-free survival (measured in days) relative to miR-331 expression. miR-331 levels were able to group patients in Gleason Grade Group 2 into subgroups in terms of possibility of biochemical failure.

FIG. 5 shows a graphical representation of cumulative and progression-free survival (measured in days) relative to (A) miR-29a, (B) miR-664a, or (C) miR-151-3p expression. Three serum exosomal miRNAs were able to distinguish patients in Gleason Grade Group 3 into distinct prognostic groups (N=56).

FIG. 6 shows a graphical representation of cumulative and progression-free survival (measured in days) relative to (A) miR-29a in Gleason Grade Groups 1+2 (N=374), (B) miR-664a in Gleason Grade Groups 1+2 (N=374), (C) miR-29a in Gleason Grade Groups 2+3 (N=224), and (D) miR-664a in Gleason Grade Groups 2+3 (N=224). Serum exosomal miRNAs had the ability to stratify patients in combined Gleason Grade Group 1+2 and 2+3.

FIG. 7 shows (A) serum preoperative PSA was not able to predict patients in Gleason Grade Group 1 from higher grade groups; and (B) serum PSA did not have the ability to distinguish patients with smaller tumors volume from bigger ones (cut-off=5%).

FIG. 8 shows (A) serum exosomal miR-29a is useful in distinguishing Gleason Grade Group 1 from higher grade groups; (B-C) the combined models for distinguishing Gleason Grade Group 1 from higher grade groups; (D) serum exosomal miR-29a could be used to distinguish indolent tumors from aggressive ones; and (E-F) combined serum exosomal miRNAs improve the ability to predict indolent tumors and distinguish from aggressive ones.

FIG. 9 shows urinary exosomal miRNAs were able to group patients into distinct prognostic groups (normalized by miR-29a) according to individual expression of (A) miR-590-5p, (B) miR-331, (C) miR-19a, (D) miR-374-5p, (E) miR-195, (F) miR-26b, (G) miR-29c, (H) miR-191, (I) miR-378, (J) miR-454, and (K) miR-99a.

FIG. 10 shows miR-590-5p is useful in grouping patients in Gleason Grade Group 2 into subgroups in terms of possibility of biochemical failure (normalized by miR-29a; N=168).

FIG. 11 shows urinary exosomal miRNAs are useful in distinguishing patients in Gleason Grade Group 3 into distinct prognostic groups (normalized by miR-29a; N=56) according to (A) miR-590-5P expression, (B) miR-195 expression, (C) miR-374-5p expression, and (D) miR-26b expression.

FIG. 12 shows urinary exosomal miRNAs are useful in stratifying patients in combined Gleason Grade Group 1+2 and 2+3 (normalized by miR-29a; N=224) according to (A) miR-590-5p expression, (B) miR-195 expression, (C) miR-374-5p expression, (D) miR-331 expression, (E) miR-590-5p expression, (F) miR-195 expression, (G) miR-374-5p expression, (H) miR-26b expression, and (I) miR-331 expression.

FIG. 13 shows urinary exosomal miRNAs are useful in grouping patients into distinct prognostic groups (normalized by creatinine) according to (A) miR-374-5p expression, (B) miR-99a expression, (C) miR-590-5p expression and (D) miR-331 expression.

FIG. 14 shows miR-590-5p and miR-374-5p are useful in grouping patients in Gleason Grade Group 3 into subgroups in terms of possibility of biochemical failure (normalized by creatinine) according to (A) miR-590-5p expression (N=56), and (B) miR-374-5p expression (N=56).

FIG. 15 shows urinary exosomal miRNAs are useful in stratifying patients in combined Gleason Grade Group 1+2 and 2+3 (normalized by creatinine) according to (A) miR-331 expression (Gleason Grade Group 1+2; N=374), (B) miR-374-5p expression (Gleason Grade Group 1+2; N=374), (C) miR-590-5p expression (Gleason Grade Group 1+2; N=374), (D) miR-331 expression (Gleason Grade Group 2+3; N=224), and (E) miR-374-5p expression (Gleason Grade Group 2+3; N=224).

FIG. 16 shows comparisons between serum preoperative PSA, combined models with serum and urinary exosomal miRNAs in terms of distinguishing Gleason Grade Groups from others in the cohort of patients with PSA≥1 ng/ml. (A) Use of serum PSA was not able to predict Gleason Grade Group 1 from higher grade groups (Gleason Grade Groups 2 to 5); (B) Serum miR-29a is useful in distinguishing patients in Gleason Grade Group 1 from others (Gleason Grade Groups 2 to 5); (C) Combined PSA with serum miR-29a is useful in stratifying patients into low grade group and higher grade groups (Gleason Grade Groups 2 to 5); (D) Combining PSA, serum miR-29a and urinary miR-26b (normalized by miR-29a) is useful in distinguishing patients with Gleason Grade Group 1 from others (Gleason Grade Groups 2 to 5); and (E) Combining PSA, serum miR-29a and urinary miR-26b (normalized by creatinine) is useful in distinguishing patients with Gleason Grade Group 1 from others (Gleason Grade Groups 2 to 5).

FIG. 17 shows combining PSA, serum miR-29a and urinary miR-26b normalized by miR-29a (A), or creatinine (B), is useful in predicting patients with indolent tumors from aggressive ones in the cohort with PSA1 ng/ml.

FIG. 18 shows combining PSA, serum miR-29a and urinary miR-26b normalized by miR-29a (A), or creatinine (B), is useful in predicting patients in Gleason Grade Group 1 from higher grade groups (Gleason Grade Groups 2 to 5) in the cohort with PSA1 ng/ml and creatinine level range of 10-90%.

FIG. 19 shows combining PSA, serum miR-29a and urinary miR-26b normalized by miR-29a (A), or creatinine (B), is useful in predicting indolent tumors from aggressive ones in the cohort with PSA1 ng/ml as well as creatinine range 10-90%.

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise. The modifier “about” used in connection with a quantity is inclusive of the stated value and has the meaning dictated by the context (e.g., it includes the degree of error associated with measurement of the particular quantity). When referring to a period such as about a year or annually, it includes a range from 9 months to 15 months. All ranges disclosed herein are inclusive of the endpoints, and the endpoints are independently combinable with each other.

The term “subject”, as used herein, refers to any individual who is the target of monitoring, administration or treatment. The subject can be an animal, for example, a mammal, optionally a human. The term “patient” refers to a subject under the monitoring, care or treatment of a health care professional.

The terms “MicroRNA” and “miRNA” as used herein refer to short, single-stranded RNA molecules approximately 21-23 nucleotides in length which are partially complementary to one or more mRNA molecules (target mRNAs). MiRNAs down-regulate gene expression by inhibiting translation or by targeting the mRNA for degradation or deadenylation. MiRNAs base-pair with miRNA recognition elements (MREs) located on their mRNA targets, usually on the 3′-UTR, through a region called the ‘seed region’ which includes nucleotides 2-8 from the 5′-end of the miRNA. Matches between a miRNA and its target are generally asymmetrical. The complementarity of seven or more bases to the 5′-end miRNA has been found to be sufficient for regulation. The information about miRNAs as disclosed herein are shown in Table 1A.

TABLE 1A
miRNA sequence information and accession numbers.

| | Mature | Mature miRNA | | | |
|---|---|---|---|---|---|
| Mature | miRNA | miRBase | Stem- | | Stem-loop |
| miRNA | Sequences | Accession | loop | | Accession |
| Name | (5′ to 3′) | Number | ID | Stem-loop detail | Number |
| has- | UGUGCAAA | MIMAT0000073 | hsa- | GCAGUCCUCUGUUAGUUU | MI0000073 |
| miR- | UCUAUGCA | | mir- | UGCAUAGUUGCACUACAA | |
| 19a | AAACUGA | | 19a | GAAGAAUGUAGUUGUGCA | |
| | [SEQ ID | | | AAUCUAUGCAAAACUGAU | |
| | NO: 1] | | | GGUGGCCUGC | |
| | | | | [SEQ ID NO: 24] | |
| Has- | UGUGCAAA | MIMAT0000074 | hsa- | CACUGUUCUAUGGUUAGU | MI0000074 |
| miR- | UCCAUGCA | | mir- | UUUGCAGGUUUGCAUCCA | |
| 19b | AAACUGA | | 19b-1 | GCUGUGUGAUAUUCUGCU | |
| | [SEQ ID | | | GUGCAAAUCCAUGCAAAA | |
| | NO: 2] | | | CUGACUGUGGUAGUG | |
| | | | | [SEQ ID NO: 25] | |
| | | | hsa- | ACAUUGCUACUUACAAUU | MI0000075 |
| | | | mir- | AGUUUUGCAGGUUUGCAU | |
| | | | 19b-2 | UUCAGCGUAUAUAUGUAU | |
| | | | | AUGUGGCUGUGCAAAUCC | |
| | | | | AUGCAAAACUGAUUGUGA | |
| | | | | UAAUGU | |
| | | | | [SEQ ID NO: 26] | |
| hsa- | UAGCACCA | MIMAT0000086 | hsa- | AUGACUGAUUUCUUUUGG | MI0000087 |
| miR- | UCUGAAAU | | mir- | UGUUCAGAGUCAAUAUAA | |
| 29a | CGGUUA | | 29a | UUUUCUAGCACCAUCUGA | |
| | [SEQ ID | | | AAUCGGUUAU | |
| | NO: 3] | | | [SEQ ID NO: 27] | |
| Has- | UGUAAACA | MIMAT0000244 | hsa- | ACCAUGCUGUAGUGUGUG | MI0000736 |
| miR- | UCCUACAC | | mir- | UAAACAUCCUACACUCUC | |
| 30c | UCUCAGC | | 30c-1 | AGCUGUGAGCUCAAGGUG | |
| | [SEQ ID | | | GCUGGGAGAGGGUUGUU | |
| | NO: 4] | | | UACUCCUUCUGCCAUGGA | |
| | | | | [SEQ ID NO: 28] | |
| | | | hsa- | AGAUACUGUAAACAUCCU | MI0000254 |
| | | | mir- | ACACUCUCAGCUGUGGAA | |
| | | | 30c-2 | AGUAAGAAAGCUGGGAGA | |
| | | | | AGGCUGUUUACUCUUUCU | |
| | | | | [SEQ ID NO: 29] | |
| Has- | UGGCAGU | MIMAT0000255 | hsa- | GGCCAGCUGUGAGUGUUU | MI0000268 |
| miR- | GUCUUAG | | mir- | CUUUGGCAGUGUCUUAGC | |
| 34a | CUGGUUG | | 34a | UGGUUGUUGUGAGCAAUA | |
| | U | | | GUAAGGAAGCAAUCAGCA | |
| | [SEQ ID | | | AGUAUACUGCCCUAGAAG | |
| | NO: 5] | | | UGCUGCACGUUGUGGGG | |
| | | | | CCC | |
| | | | | [SEQ ID NO: 30] | |
| hsa- | UUAAGGCA | MI0002762 | age- | AUCAAGAUCAGAGGCUCU | MI0002762 |
| miR- | CGCGGUG | | mir- | GCCCUCCGUGUUCACAGC | |
| 124a | AAUGCCA | | 124a | GGACCUUGAUUUAAUGUC | |
| | [SEQ ID | | | AUACAAUUAAGGCACGCG | |
| | NO: 6] | | | GUGAAUGCCAAGAGCGGA | |
| | | | | GCCUACGGCUGCACUUG | |
| | | | | [SEQ ID NO: 31] | |
| hsa- | UUUGGUC | MIMAT0000427 | hsa- | ACAAUGCUUUGCUAGAGC | MI0000450 |
| miR- | CCCUUCAA | | mir- | UGGUAAAAUGGAACCAAA | |
| 133a | CCAGCUG | | 133a- | UCGCCUCUUCAAUGGAUU | |
| | [SEQ ID | | 1 | UGGUCCCCUUCAACCAGC | |
| | NO: 7] | | | UGUAGCUAUGCAUUGA | |
| | | | | [SEQ ID NO: 32] | |
| | | | hsa- | GGGAGCCAAAUGCUUUGC | MI0000451 |
| | | | mir- | UAGAGCUGGUAAAAUGGA | |
| | | | 133a- | ACCAAAUCGACUGUCCAA | |
| | | | 2 | UGGAUUUGGUCCCCUUCA | |
| | | | | ACCAGCUGUAGCUGUGCA | |
| | | | | UUGAUGGCGCCG | |
| | | | | [SEQ ID NO: 33] | |
| hsa- | UUUGGUC | MIMAT0000770 | hsa- | CCUCAGAAGAAAGAUGCC | MI0000822 |
| miR- | CCCUUCAA | | mir- | CCCUGCUCUGGCUGGUCA | |
| 133b | CCAGCUA | | 133b | AACGGAACCAAGUCCGUC | |
| | [SEQ ID | | | UUCCUGAGAGGUUUGGUC | |
| | NO: 8] | | | CCCUUCAACCAGCUACAG | |
| | | | | CAGGGCUGGCAAUGCCCA | |
| | | | | GUCCUUGGAGA | |
| | | | | [SEQ ID NO: 34] | |
| hsa- | CUAGACUG | MIMAT0000757 | hsa- | UUUCCUGCCCUCGAGGAG | MI0000809 |
| miR- | AAGCUCCU | | mir- | CUCACAGUCUAGUAUGUC | |
| 151-3p | UGAGG | | 151a | UCAUCCCCUACUAGACUG | |
| | [SEQ ID | | | AAGCUCCUUGAGGACAGG | |
| | NO: 9] | | | GAUGGUCAUACUCACCUC | |
| | | | | [SEQ ID NO: 35] | |
| hsa- | CAACGGAA | MIMAT0000440 | hsa- | CGGCUGGACAGCGGGCAA | MI0000465 |
| miR- | UCCCAAAA | | mir- | CGGAAUCCCAAAAGCAGC | |
| 191 | GCAGCUG | | 191 | UGUUGUCUCCAGAGCAUU | |
| | [SEQ ID | | | CCAGCUGCGCUUGGAUUU | |
| | NO: 10] | | | CGUCCCCUGCUCUCCUGC | |
| | | | | CU | |
| | | | | [SEQ ID NO: 36] | |
| hsa- | GCCCCUG | MIMAT0000760 | hsa- | GAGUUUGGUUUUGUUUG | MI0000812 |
| miR- | GGCCUAU | | mir- | GGUUUGUUCUAGGUAUGG | |
| 331 | CCUAGAA | | 331 | UCCCAGGGAUCCCAGAUC | |
| | [SEQ ID | | | AAACCAGGCCCCUGGGCC | |
| | NO: 11] | | | UAUCCUAGAACCAACCUA | |
| | | | | AGCUC | |
| | | | | [SEQ ID NO: 37] | |
| hsa- | GGCAGGU | MIMAT0003335 | hsa- | GUGUAGUAGAGCUAGGAG | MI0003681 |
| miR- | UCUCACCC | | mir- | GAGAGGGUCCUGGAGAAG | |
| 657 | UCUCUAG | | 657 | CGUGGACCGGUCCGGGU | |
| | G | | | GGGUUCCGGCAGGUUCU | |
| | [SEQ ID | | | CACCCUCUCUAGGCCCCA | |
| | NO: 12] | | | UUCUCCUCUG | |
| | | | | [SEQ ID NO: 38] | |
| hsa- | UAUUCAUU | MIMAT0005949 | hsa- | GAACAUUGAAACUGGCUA | MI0006442 |
| miR- | UAUCCCCA | | mir- | GGGAAAAUGAUUGGAUAG | |
| 664 | GCCUACA | | 664a | AAACUAUUAUUCUAUUCA | |
| | [SEQ ID | | | UUUAUCCCCAGCCUACAA | |
| | NO: 13] | | | AAUGAAAAAA | |
| | | | | [SEQ ID NO: 39] | |
| hsa- | CCUGGAAA | MIMAT0004923 | hsa- | UUAGUGGUACUAUACCUC | MI0005541 |
| miR- | CACUGAG | | mir- | AGUUUUAUCAGGUGUUCU | |
| 875-3p | GUUGUG | | 875 | UAAAAUCACCUGGAAACA | |
| | [SEQ ID | | | CUGAGGUUGUGUCUCACU | |
| | NO: 14] | | | GAAC | |
| | | | | [SEQ ID NO: 40] | |
| hsa- | UAGCAGCA | MIMAT0000461 | hsa- | AGCUUCCCUGGCUCUAGC | MI0000489 |
| miR- | CAGAAAUA | | mir- | AGCACAGAAAUAUUGGCA | |
| 195 | UUGGC | | 195 | CAGGGAAGCGAGUCUGCC | |
| | [SEQ ID | | | AAUAUUGGCUGUGCUGCU | |
| | NO: 15] | | | CCAGGCAGGGUGGUG | |
| | | | | [SEQ ID NO: 41] | |
| hsa- | AUAUAAUA | MIMAT0004955 | hsa- | ACUCGGAUGGAUAUAAUA | MI0005566 |
| miR- | CAACCUGC | | mir- | CAACCUGCUAAGUGUCCU | |
| 374-5p | UAAGUG | | 374b | AGCACUUAGCAGGUUGUA | |
| | [SEQ ID | | | UUAUCAUUGUCCGUGUCU | |
| | NO: 16] | | | [SEQ ID NO: 42] | |
| hsa- | GAGCUUAU | MIMAT0003258 | hsa- | UAGCCAGUCAGAAAUGAG | MI0003602 |
| miR- | UCAUAAAA | | mir- | CUUAUUCAUAAAAGUGCA | |
| 590-5p | GUGCAG | | 590 | GUAUGGUGAAGUCAAUCU | |
| | [SEQ ID | | | GUAAUUUUAUGUAUAAGC | |
| | NO: 17] | | | UAGUCUCUGAUUGAAACA | |
| | | | | UGCAGCA | |
| | | | | [SEQ ID NO: 43] | |
| hsa- | UUCAAGUA | MIMAT0000083 | hsa- | CCGGGACCCAGUUCAAGU | MI0000084 |
| miR- | AUUCAGGA | | mir- | AAUUCAGGAUAGGUUGUG | |
| 26b | UAGGU | | 26b | UGCUGUCCAGCCUGUUCU | |
| | [SEQ ID | | | CCAUUACUUGGCUCGGGG | |
| | NO: 18] | | | ACCGG | |
| | | | | [SEQ ID NO: 44] | |
| hsa- | UAAUGCCC | MIMAT0000710 | hsa- | ACCGCAGGGAAAAUGAGG | MI0000767 |
| miR- | CUAAAAAU | | mir- | GACUUUUGGGGGCAGAUG | |
| 365 | CCUUAU | | 365a | UGUUUCCAUUCCACUAUC | |
| | [SEQ ID | | | AUAAUGCCCCUAAAAAUC | |
| | NO: 19] | | | CUUAUUGCUCUUGCA | |
| | | | | [SEQ ID NO: 45] | |
| | | | hsa- | AGAGUGUUCAAGGACAGC | MI0000769 |
| | | | mir- | AAGAAAAAUGAGGGACUU | |
| | | | 365b | UCAGGGGCAGCUGUGUUU | |
| | | | | UCUGACUCAGUCAUAAUG | |
| | | | | CCCCUAAAAAUCCUUAUU | |
| | | | | GUUCUUGCAGUGUGCAUC | |
| | | | | GGG | |
| | | | | [SEQ ID NO: 46] | |
| hsa- | UAGCACCA | MIMAT0000681 | hsa- | AUCUCUUACACAGGCUGA | MI0000735 |
| miR- | UUUGAAAU | | mir- | CCGAUUUCUCCUGGUGUU | |
| 29c | CGGUUA | | 29c | CAGAGUCUGUUUUUGUCU | |
| | [SEQ ID | | | AGCACCAUUUGAAAUCGG | |
| | NO: 20] | | | UUAUGAUGUAGGGGGA | |
| | | | | [SEQ ID NO: 47] | |
| hsa- | AACCCGUA | MIMAT0000097 | hsa- | CCCAUUGGCAUAAACCCG | MI0000101 |
| miR- | GAUCCGAU | | mir- | UAGAUCCGAUCUUGUGGU | |
| 99a | CUUGUG | | 99a | GAAGUGGACCGCACAAGC | |
| | [SEQ ID | | | UCGCUUCUAUGGGUCUGU | |
| | NO: 21] | | | GUCAGUGUG | |
| | | | | [SEQ ID NO: 48] | |
| hsa- | CUCCUGAC | MIMAT0000731 | hsa- | AGGGCUCCUGACUCCAGG | MI0000786 |
| miR- | UCCAGGU | | mir- | UCCUGUGUGUUACCUAGA | |
| 378 | CCUGUGU | | 378a | AAUAGCACUGGACUUGGA | |
| | [SEQ ID | | | GUCAGAAGGCCU | |
| | NO: 22] | | | [SEQ ID NO: 49] | |
| hsa- | UAGUGCAA | MIMAT0003885 | hsa- | UCUGUUUAUCACCAGAUC | MI0003820 |
| miR- | UAUUGCUU | | mir- | CUAGAACCCUAUCAAUAU | |
| 454 | AUAGGGU | | 454 | UGUCUCUGCUGUGUAAAU | |
| | [SEQ ID | | | AGUUCUGAGUAGUGCAAU | |
| | NO: 23] | | | AUUGCUUAUAGGGUUUUG | |
| | | | | GUGUUUGGAAAGAACAAU | |
| | | | | GGGCAGG | |
| | | | | [SEQ ID NO: 50] | |

MiRNAs are first transcribed as primary transcripts (pri-miRNA) by RNA polymerase II or RNA polymerase III. Generally, a pri-miRNA comprises a double stranded stem of about 33 base pairs, a terminal loop and two flanking unstructured single-stranded segments. Pri-miRNA is processed by a protein complex which consists of an RNase III enzyme (Drosha), and a double stranded-RNA binding protein (DGCR8 or DiGeorge syndrome critical region 8 gene) resulting in a short 70-nucleotide stem-loop structure called pre-miRNA. The pre-miRNA is transported from the nucleus to the cytoplasm by Exportin-5 (Exp-5) by the action of RanGTPase. In the cytoplasm, Dicer (an RNAse III endonuclease) cleaves the pre-miRNAs into short RNA duplexes termed miRNA duplexes. After cleavage, the miRNA duplex is unwound by an RNA helicase and the mature miRNA strand binds to its target mRNAs, and the complementary strand (i.e. passenger strand) is degraded.

“Pre-miRNA” or “pre-miR” refers to a short 70-nucleotide stem-loop structure processed from a pri-miRNA. A pre-miRNA comprises a stem or double stranded region (i.e., a region of a nucleic acid molecule that is in a double stranded conformation via hydrogen bonding between the nucleotides) and a loop region of unpaired nucleotides at the terminal end of the stem. The double stranded region includes the mature miRNA sequence (that binds to a target mRNA) hydrogen bonded to its complementary sequence.

The present inventors have determined that it is possible to select a prostate cancer patient preoperative of prostatectomy with known PSA level for active surveillance by comparing similarity between a biological sample exosomal miRNA profile to one or more control profiles. While aggressive form of prostate cancer requiring immediate surgery may be readily identified, a vast majority (˜90%) of prostate cancer has indeterminate diagnosis. Patients with indeterminate diagnosis have the options of active surveillance and prostatectomy. However, there is no current test to accurately and reliably offer these options. Decisions are made on a number of clinical criteria that are not always accurate. The term “active surveillance”, as used herein, refers to a clinical option for prostate cancer that is offered to appropriate patients, as identified by the methods described herein, who would also be candidates for prostatectomy if the disease progresses. Active surveillance offers men with a prostate cancer that is considered to be indolent or on a non-progressive course, such that it has a low risk of causing harm in the absence of treatment, a chance to delay or avoid aggressive treatment such as prostatectomy and its associated side effects. Active surveillance as described by the methods disclosed herein involves clinical assessment that includes annual exosomal miRNA and PSA measurement, and optionally monitoring by imaging, for men who are preoperative of prostatectomy. Levels of exosomal miRNAs are determined or measured from biological samples, for example from a liquid biopsy, such as serum or urine. Invasive procedure such as prostate biopsy is avoided.

Accordingly, the present inventors have provide a method for monitoring prostate cancer progression in a patient preoperative of prostatectomy with a known age and/or PSA level, comprising:
(a) (i) determining or measuring exosomal miRNA levels in a biological sample to provide a biological sample exosomal miRNA profile comprising exosomal miRNA level of at least one, at least two, or at least three exosomal miRNA from miR-19a, miR-19b, miR-29a, miR-30c, miR-34a, miR-124a, miR-133a, miR-133b, miR-151-3p, miR-191, miR-331, miR-657, miR-664a and miR-875-3p; or (ii) determining or measuring exosomal miRNA levels in a biological sample to provide a biological sample exosomal miRNA profile comprising exosomal miRNA level of at least three exosomal miRNA from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a prostatectomy control profile; and/or (iii) a higher level of similarity to an active surveillance control profile than to a prostatectomy control profile indicates low disease aggressiveness; or wherein (iv) a low level of similarity of the sample exosomal miRNA profile to an active surveillance control profile; (v) a high level of similarity to a prostatectomy control profile; and/or (vi) a lower level of similarity to an active surveillance control profile than to a prostatectomy control profile indicates high disease aggressiveness.

In an embodiment, the patient has a known age and known PSA level. In an embodiment, the patient has a known age. In an embodiment, the patient has a known PSA level.

In an embodiment, step (a) comprises determining or measuring exosomal miRNA levels in a biological sample to provide a biological sample exosomal miRNA profile comprising exosomal miRNA level of at least three exosomal miRNA from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p. In another embodiment, step (a) comprises determining or measuring exosomal miRNA levels in a biological sample to provide a biological sample exosomal miRNA profile comprising exosomal miRNA level of at least one, at least two, or at least three exosomal miRNA from miR-19a, miR-19b, miR-29a, miR-30c, miR-34a, miR-124a, miR-133a, miR-133b, miR-151-3p, miR-191, miR-331, miR-657, miR-664a and miR-875-3p. In another embodiment, step (a) comprises determining or measuring exosomal miRNA levels in a biological sample to provide a biological sample exosomal miRNA profile comprising biological sample exosomal miRNA profile comprising at least three serum exosomal miRNA selected from miR-29a, miR-34a, miR-331, miR-664a, and miR-875-3p, and/or at least three urine exosomal miRNA selected from miR-26b, miR-29c, miR-99a, miR-195, miR-331, miR-374-5p, miR-454, and miR-590-5p. In another embodiment, step (a) comprises determining or measuring a biological sample exosomal miRNA profile comprising at least three serum exosomal miRNA selected from miR-19b, miR-34a, miR-664a, and miR-875-3p, and at least three urine exosomal miRNA selected from miR-34a, miR-99a, miR-133b, miR-331, miR-374-5p, and miR-454. In another embodiment, step (a) comprises determining or measuring exosomal miRNA levels in a biological sample to provide a biological sample exosomal miRNA profile comprising exosomal miRNA level of miR-19a, miR-19b, miR-29a, miR-30c, miR-34a, miR-124a, miR-133a, miR-133b, miR-151-3p, miR-191, miR-331, miR-657, miR-664a and miR-875-3p. In another embodiment, step (a) comprises determining or measuring exosomal miRNA levels in a biological sample to provide a biological sample exosomal miRNA profile comprising biological sample exosomal miRNA profile comprising serum exosomal miRNA miR-29a, miR-34a, miR-331, miR-664a, and miR-875-3p, and/or at least three urine exosomal miRNA selected from miR-26b, miR-29c, miR-99a, miR-195, miR-331, miR-374-5p, miR-454, and miR-590-5p. In another embodiment, step (a) comprises determining or measuring a biological sample exosomal miRNA profile comprising serum exosomal miRNA miR-19b, miR-34a, miR-664a, and miR-875-3p, and urine exosomal miRNA miR-34a, miR-99a, miR-133b, miR-331, miR-374-5p, and miR-454.

In an embodiment, the biological sample is a liquid biopsy, optionally a serum or urine sample.

Determination of levels of exosomal miRNA allows biological insight into aggressiveness of prostate cancer. miRNAs are important gene regulatory elements that are present in stable forms in serum and urine which may be used as non-invasive biomarkers for prostate cancer diagnosis. Without wishing to be bound by theory, exosomes are said to function as delivery vehicles of circulating exosomal miRNAs and transport them from primary cancer sites to other sites while also shielding exosomal miRNAs from nucleases. Thus, measurements from exosomal miRNAs are more reproducible than freely circulating miRNA which may be subject to degradation. The heterogeneity of prostate cancers poses challenges for existing methods in distinguishing intermediate grades of prostate cancer as aggressive or indolent. In the present disclosure, determination of the expression levels of exosomal miRNA described herein allows distinguishing indolent disease from aggressive forms.

In an embodiment, the determining or measuring exosomal miRNA levels in a biological sample to provide the biological sample exosomal miRNA profile comprising determining or measuring:
(a) (i) at least two serum exosomal miRNAs selected from miR-29a, miR-30c, miR-151-3p, miR-191 and miR-664a, or (ii) at least two urine exosomal miRNAs selected from miR-19a, miR-195, miR-331, miR-374-5p and miR-590-5p;
(b) (i) at least three serum exosomal miRNAs selected from miR-19a, miR-29a, miR-151-3p and miR-664a; or (ii) at least three urine exosomal miRNAs selected from miR-19a, miR-26b, miR-331, miR-374-5p and miR-590-5p; and/or
(c) (i) at least two serum exosomal miRNAs selected from miR-29a, miR-133a, miR-151-3p and miR-657; or (ii) at least one serum exosomal miRNA selected from miR-19a, miR-133a, miR-151-3p and miR-657, and at least one urine exosomal miRNA selected from miR-19a, miR-26a, miR-331 and miR-590.

In an embodiment, the biological sample exosomal miRNA profile comprises serum exosomal miRNAs miR-29a, miR-30c, miR-151-3p, miR-191 and miR-664a. In another embodiment, the biological sample exosomal miRNA profile comprises serum exosomal miRNAs urine exosomal miRNAs miR-19a, miR-195, miR-331, miR-374-5p and miR-590-5p. In another embodiment, the biological sample exosomal miRNA profile comprises urine exosomal miRNAs miR-19a, miR-195, miR-331, miR-374-5p and miR-590-5p. In another embodiment, the biological sample exosomal miRNA profile comprises serum exosomal miRNAs miR-19a, miR-29a, miR-151-3p and miR-664a. In another embodiment. In another embodiment, the biological sample exosomal miRNA profile comprises urine exosomal miRNAs miR-19a, miR-26b, miR-331, miR-374-5p and miR-590-5p. In another embodiment, the biological sample exosomal miRNA profile comprises serum exosomal miRNAs miR-29a, miR-133a, miR-151-3p and miR-657. In another embodiment, the biological sample exosomal miRNA profile comprises serum exosomal miRNA miR-19a, miR-133a, miR-151-3p and miR-657 and urine exosomal miRNA miR-19a, miR-26a, miR-331 and miR-590. In another embodiment, the biological sample exosomal miRNA profile and the one or more control profiles comprise (i) the levels of at least three serum exosomal miRNAs selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p, and/or (ii) at least three urine exosomal miRNA selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p.

In another embodiment, the methods for monitoring prostate cancer progression in a patient preoperative of prostatectomy with a known age and/or PSA level comprises the steps:
(a) providing at least one biological sample from a patient, wherein the biological sample is serum or urine;
(b) determining a biological sample profile comprising exosomal miRNA levels;
(c) determining or measuring the level of similarity of said biological sample exosomal miRNA profile to one or more control profiles, wherein (i) a high level of similarity of the sample profile to an active surveillance control profile; (ii) a low level of similarity to a prostatectomy control profile; and/or (iii) a higher level of similarity to an active surveillance control profile than to a prostatectomy control profile indicates low disease aggressiveness; or wherein (iv) a low level of similarity of the sample profile to an active surveillance control profile; (v) a high level of similarity to a prostatectomy control profile; and/or (vi) a lower level of similarity to an active surveillance control profile than to a prostatectomy control profile indicates high disease aggressiveness;
(d) selecting said patient for active surveillance when said patient has low disease aggressiveness, or selecting said patient for prostatectomy when said patient has high disease aggressiveness; and
(e) repeating (a)-(d) in about one year when said patient is selected for active surveillance.

In an embodiment, the patient has a known age and known PSA level. In an embodiment, the patient has a known age. In an embodiment, the patient has a known PSA level.

The skilled person readily recognizes the use of Receiver Operating Characteristic (ROC) analysis in determining probabilities (P) in the methods described herein. For ROC analysis with two or more independent variables, the probabilities (P) using multivariable logistic regression model was calculated. Afterwards, probability (P) value was used as new independent variables in ROC analysis. The risk scores for individual patients were calculated using xβ, i.e. the cut-off value (P) was calculated using the formula P=1/[1+exp.(−xβ)]=exp.(xβ)/[1+exp.(xβ)], where xβ is standard linear form in multivariable logistic regression analysis. Accordingly, in an embodiment, the probabilities (P) were used for ROC analysis.

The cut-off (P) value as shown in the present disclosure was calculated according to Youden Index (maximum value of (Sensitivity+Specificity−1)), which captures the performance of a dichotomous diagnostic test. However, the skilled person may find that when a cut-off value is set at Youden Index, the dependency on sensitivity or specificity may require adjustment. Hence, the skilled person will alternately adjust the cut-off value according to clinical experience. For example, to balance between specificity and sensitivity, the skilled person may increase specificity while decrease sensitivity if biomarkers lack high specificity.

Accordingly, the methods described herein also generate a numerical score based on biological sample exosomal miRNA profile. For example, in an embodiment, a sample or control profile can generate a numerical score based on biological sample exosomal miRNA profile calculated according to the following formula:

P=1/[1+exp.(−xβ)]=exp.(xβ)/[1+exp.(xβ)]
where xβ is standard linear form in multivariable logistic regression analysis,
wherein the numerical score is used in Receiver Operating Characteristic (ROC) analysis.

The methods described herein can also predict or identify biochemical failure or biochemical recurrence, i.e. disease relapse, that will or is likely to occur after prostatectomy and/or radiation in a patient. In an embodiment, the determining or measuring exosomal miRNA levels in a biological sample to provide a biological sample exosomal miRNA profile predicts biochemical failure of a patient, and allow treatment such as prostatectomy or radical prostatectomy.

Accordingly, also provided is a method for predicting biochemical failure in a prostate cancer patient preoperative of prostatectomy, comprising:
(a) determining or measuring exosomal miRNA levels in a biological sample to provide a biological sample exosomal miRNA profile comprising (i) at least two serum exosomal miRNAs selected from miR-29a, miR-30c, miR-151-3p, miR-191 and miR-664a, or (ii) at least two urine exosomal miRNAs selected from miR-19a, miR-195, miR-331, miR-374-5p and miR-590-5p; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure.

In an embodiment, the biological sample exosomal miRNA profile comprises serum exosomal miRNAs miR-29a, miR-30c, miR-151-3p, miR-191 and miR-664a. In another embodiment, the biological sample exosomal miRNA profile comprises urine exosomal miRNAs miR-19a, miR-195, miR-331, miR-374-5p and miR-590-5p.

In an embodiment, predicting biochemical failure in a prostate cancer patient preoperative of prostatectomy comprises determining or measuring exosomal miRNA levels in a biological sample to provide the biological sample exosomal miRNA profile, further comprises providing at least one biological sample from a patient, wherein the biological sample is serum or urine, and repeating steps (a) and (b) in about one year when said patient has indolent disease.

The Gleason System separates architectural features from biopsy specimen into 1 of 5 histological patterns. These are in decreasing differentiation order but increasing in number: Pattern 1 is the most differentiated and pattern 5 is the least differentiated. A newly published grade grouping system adopted by the International Society of Urologic Pathology (ISUP) groups different scores into a 5-tier system with prognostic significance as follows: Gleason score 3+3=grade group 1; Gleason score 3+4=grade group 2; Gleason score 4+3=grade group 3; Gleason score 4+4 or 3+5 or 5+3=grade group 4; Gleason score >8=grade group 5, where the first Gleason score value is the most prevalent architectural pattern, and the second Gleason score value is the second most prevalent pattern. The methods described herein can also predict or identify biochemical failure or biochemical recurrence, i.e. disease relapse, for a patient classified under Gleason Grade Group as having grade group 1, 2, 3, or 4 prostate cancer. In an embodiment, the determining or measuring of exosomal miRNA levels identifies biochemical failure of a patient, wherein said patient is classified under Gleason Grade Group as having grade 1, 2, 3, or 4 prostate cancer.

Accordingly, also provided is a method for predicting biochemical failure in a prostate cancer patient preoperative of prostatectomy and is classified under Gleason Grade Group as having grade 1, 2, 3, or 4 prostate cancer, comprising:
(a) determining or measuring exosomal miRNA levels in a biological sample to provide a biological sample exosomal miRNA profile comprising (i) at least three serum exosomal miRNAs selected from miR-19a, miR-29a, miR-151-3p and miR-664a; or (ii) at least three urine exosomal miRNAs selected from miR-19a, miR-26b, miR-331, miR-374-5p and miR-590-5p; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure.

In an embodiment, predicting biochemical failure in a prostate cancer patient preoperative of prostatectomy and is classified under Gleason Grade Group as having grade 1, 2, 3, or 4 prostate cancer comprises the determining or measuring exosomal miRNA levels in a biological sample to provide the biological sample exosomal miRNA profile, further comprises providing at least one biological sample from a patient, wherein the biological sample is serum or urine, and repeating steps (a) and (b) in about one year when said patient has indolent disease.

Presently described methods can also predict biochemical failure in a prostate cancer patient preoperative of prostatectomy who has been classified under a specific Gleason Grade Group.

Accordingly, also provided is a method for predicting biochemical failure in a prostate cancer patient preoperative of prostatectomy and is classified under Gleason Grade Group as having grade 1 prostate cancer, comprising:
(a) determining or measuring exosomal miRNA levels in a biological sample to provide a biological sample exosomal miRNA profile comprising (i) at least one of serum exosomal miRNAs miR-29a, miR-664a and miR-151-3p; (ii) serum exosomal miRNA miR-29a; (iii) serum exosomal miRNAs miR-657 and miR-151-3p, or (iv) serum exosomal miRNAs miR-133a and miR-151-3p; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure.

In an embodiment, predicting biochemical failure in a prostate cancer patient preoperative of prostatectomy and is classified under Gleason Grade Group as having grade 1 prostate cancer comprises determining or measuring exosomal miRNA levels in a biological sample to provide the biological sample exosomal miRNA profile, further comprising providing at least one biological sample from a patient, wherein the biological sample is serum or urine, and repeating steps (a) and (b) in about one year when said patient has indolent disease.

Also provided is a method for predicting biochemical failure in a prostate cancer patient preoperative of prostatectomy and is classified under Gleason Grade Group as having grade 2 prostate cancer, comprising:
(a) determining or measuring exosomal miRNA levels in a biological sample to provide a biological sample exosomal miRNA profile comprising (i) serum exosomal miRNAs miR-331; or (ii) urinary exosomal miRNA miR-590-5p normalized by miR-29a; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure.

In an embodiment, predicting biochemical failure in a prostate cancer patient preoperative of prostatectomy and is classified under Gleason Grade Group as having grade 2 prostate cancer comprises determining or measuring exosomal miRNA levels in a biological sample to provide the biological sample exosomal miRNA profile, further comprising providing at least one biological sample from a patient, wherein the biological sample is serum or urine, and repeating steps (a) and (b) in about one year when said patient has indolent disease.

Also provided is a method for predicting biochemical failure in a prostate cancer patient preoperative of prostatectomy and is classified under Gleason Grade Group as having grade 3 prostate cancer, comprising:
(a) determining or measuring exosomal miRNA levels in a biological sample to provide a biological sample exosomal miRNA profile comprising (i) at least one of serum exosomal miRNAs miR-29a, miR-664a, and miR-151-3p; or (ii) at least one of urinary exosomal miRNAs miR-590-5p, miR-195, miR-374-5p and miR-26b normalized by miR-29a; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure.

In an embodiment, a method for predicting biochemical failure comprises determining or measuring exosomal miRNA levels in a biological sample to provide a biological sample exosomal miRNA profile comprising serum exosomal miRNAs miR-29a, miR-664a, and miR-151-3p. In another embodiment, a method for predicting biochemical failure comprises determining or measuring exosomal miRNA levels in a biological sample to provide a biological sample exosomal miRNA profile comprising urine exosomal miRNAs miR-590-5p, miR-195, miR-374-5p and miR-26b normalized by miR-29a. In another embodiment, a method for predicting biochemical failure comprises determining or measuring exosomal miRNA levels in a biological sample to provide a biological sample exosomal miRNA profile comprising serum exosomal miRNAs miR-29a, miR-664a, and miR-151-3p, and urine exosomal miRNAs miR-590-5p, miR-195, miR-374-5p and miR-26b normalized by miR-29a.

In an embodiment, predicting biochemical failure in a prostate cancer patient preoperative of prostatectomy and is classified under Gleason Grade Group as having grade 3 prostate cancer comprises determining or measuring exosomal miRNA levels in a biological sample to provide the biological sample exosomal miRNA profile, further comprising providing at least one biological sample from a patient, wherein the biological sample is serum or urine, and repeating steps (a) and (b) in about one year when said patient has indolent disease.

The decision to actively surveil or perform prostatectomy in previous methods depends on tumor volume in the biopsy, and Gleason score of tumor biopsy specimen. The methods described herein can also predict or identify Gleason score or tumor volume of prostatectomy of a patient without performing a tumor biopsy. In an embodiment, the determining or measuring of exosomal miRNA levels identifies Gleason score or tumor volume of prostatectomy of said patient.

Accordingly, also provided is a method for predicting biochemical failure in a prostate cancer patient preoperative of prostatectomy with a known age and/or PSA level, comprising:
(a) (i) determining or measuring exosomal miRNA levels in a biological sample to provide a biological sample exosomal miRNA profile comprising at least two serum exosomal miRNAs selected from miR-29a, miR-133a, miR-151-3p and miR-657; (ii) determining or measuring exosomal miRNA levels in a biological sample to provide a biological sample exosomal miRNA profile comprising at least one serum exosomal miRNA selected from miR-19a, miR-133a, miR-151-3p and miR-657 and at least one urine exosomal miRNA selected from miR-19a, miR-26a, miR-331 and miR-590; (iii) determining or measuring exosomal miRNA levels in a biological sample to provide a biological sample exosomal miRNA profile comprising at least three serum exosomal miRNA from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p; or (iv) determining or measuring exosomal miRNA levels in a biological sample to provide a biological sample exosomal miRNA profile comprising at least three urine exosomal miRNA from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p; and
(b) determining or measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure.

In an embodiment, the patient has a known age and known PSA level. In an embodiment, the patient has a known age. In an embodiment, the patient has a known PSA level.

In an embodiment, step (a) comprises determining or measuring exosomal miRNA levels in a biological sample to provide a biological sample exosomal miRNA profile comprising at least two serum exosomal miRNAs selected from miR-29a, miR-133a, miR-151-3p and miR-657. In another embodiment, step (a) comprises determining or measuring exosomal miRNA levels in a biological sample to provide a biological sample exosomal miRNA profile comprising at least one serum exosomal miRNA selected from miR-19a, miR-133a, miR-151-3p and miR-657 and at least one urine exosomal miRNA selected from miR-19a, miR-26a, miR-331 and miR-590. In another embodiment, step (a) comprises determining or measuring exosomal miRNA levels in a biological sample to provide a biological sample exosomal miRNA profile comprising at least three serum exosomal miRNA selected from miR-29a, miR-34a, miR-331, miR-664a, and miR-875-3p, and/or at least three urine exosomal miRNA selected from miR-26b, miR-29c, miR-99a, miR-195, miR-331, miR-374-5p, miR-454, and miR-590-5p. In another embodiment, step (a) comprises determining or measuring exosomal miRNA levels in a biological sample to provide a biological sample exosomal miRNA profile comprising at least three serum exosomal miRNA selected from miR-19b, miR-34a, miR-664a, and miR-875-3p, and/or at least three urine exosomal miRNA selected from miR-34a, miR-99a, miR-133b, miR-331, miR-374-5p, and miR-454. In another embodiment, step (a) comprises determining or measuring exosomal miRNA levels in a biological sample to provide a biological sample exosomal miRNA profile comprising at least three serum exosomal miRNAs selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p. In another embodiment, step (a) comprises determining or measuring exosomal miRNA levels in a biological sample to provide a biological sample exosomal miRNA profile comprising at least three urine exosomal miRNAs selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p. In another embodiment, step (a) comprises determining or measuring exosomal miRNA levels in a biological sample to provide a biological sample exosomal miRNA profile comprising serum exosomal miRNAs from miR-29a, miR-133a, miR-151-3p and miR-657. In another embodiment, step (a) comprises determining or measuring exosomal miRNA levels in a biological sample to provide a biological sample exosomal miRNA profile comprising serum exosomal miRNA miR-133a, miR-151-3p and miR-657 and urine exosomal miRNA miR-19a, miR-26a, miR-331 and miR-590. In another embodiment, step (a) comprises determining or measuring exosomal miRNA levels in a biological sample to provide a biological sample exosomal miRNA profile comprising serum exosomal miRNA miR-29a, miR-34a, miR-331, miR-664a, and miR-875-3p, and/or urine exosomal miRNA miR-26b, miR-29c, miR-99a, miR-195, miR-331, miR-374-5p, miR-454, and miR-590-5p. In another embodiment, step (a) comprises determining or measuring exosomal miRNA levels in a biological sample to provide a biological sample exosomal miRNA profile comprising serum exosomal miRNA miR-19b, miR-34a, miR-664a, and miR-875-3p, and/or urine exosomal miRNA miR-34a, miR-99a, miR-133b, miR-331, miR-374-5p, and miR-454. In another embodiment, step (a) comprises determining or measuring exosomal miRNA levels in a biological sample to provide a biological sample exosomal miRNA profile comprising serum exosomal miRNAs miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p. In another embodiment, step (a) comprises determining or measuring exosomal miRNA levels in a biological sample to provide a biological sample exosomal miRNA profile comprising urine exosomal miRNAs miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p.

In an embodiment, predicting biochemical failure in a prostate cancer patient preoperative of prostatectomy with a known age and/or PSA level comprises determining or measuring determining or measuring exosomal miRNA levels in a biological sample to provide a biological sample exosomal miRNA profile comprising the biological sample exosomal miRNA profile, further comprising providing at least one biological sample from a patient, wherein the biological sample is serum or urine, and repeating steps (a) and (b) in about one year when said patient has indolent disease. In an embodiment, the patient has a known age and known PSA level. In an embodiment, the patient has a known age. In an embodiment, the patient has a known PSA level.

A prediction of biochemical failure means the patient is in need of immediate surgery such as prostatectomy, followed by closer follow-up after surgery. In an embodiment, a method of selecting a treatment for prostate cancer in a patient preoperative of prostatectomy with known age and/or PSA level comprises (a) predicting biochemical failure described herein, and (b) selecting said patient for prostatectomy when said patient is predicted to have biochemical failure. In another embodiment, a method of selecting a treatment for prostate cancer in a patient preoperative of prostatectomy with known age and/or PSA level comprises (a) predicting indolent disease described herein, and (b) selecting said patient for active surveillance when said patient is predicted to have indolent disease. In an embodiment, the patient has a known age and known PSA level. In an embodiment, the patient has a known age. In an embodiment, the patient has a known PSA level.

Also provided is a method for treating prostate cancer in a patient preoperative of prostatectomy with known age and/or PSA level, comprising:
(A) diagnosing said patient as a candidate for prostatectomy, comprising
   (a) providing at least one biological sample from a patient, wherein the biological sample is serum or urine;
   (b) (ii) measuring said biological sample exosomal miRNA levels comprising exosomal miRNA level of at least one, at least two, or at least three exosomal miRNA from miR-19a, miR-19b, miR-29a, miR-30c, miR-34a, miR-124a, miR-133a, miR-133b, miR-151-3p, miR-191, miR-331, miR-657, miR-664a and miR-875-3p; or (ii) measuring said biological sample exosomal miRNA levels comprising exosomal miRNA level of at least three exosomal miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p;
   (c) determining said biological sample exosomal miRNA profile comprising miRNA levels of (b); and
   (d) determining or measuring the level of similarity of said biological sample exosomal miRNA profile to one or more control profiles, wherein (i) a low level of similarity of the sample profile to an active surveillance control profile; (ii) a high level of similarity to a prostatectomy control profile; and/or (iii) a lower level of similarity to an active surveillance control profile than to a prostatectomy control profile indicates high disease aggressiveness; or wherein (iv) a high level of similarity of the sample exosomal miRNA profile to an active surveillance control profile; (v) a low level of similarity to a prostatectomy control profile; and/or (vi) a higher level of similarity to an active surveillance control profile than to a prostatectomy control profile indicates low disease aggressiveness; and
(B) removing the prostate when said patient has high disease aggressiveness, or
   monitoring prostate cancer progression when said patient has low disease aggressiveness.

In an embodiment, the patient has a known age and known PSA level. In an embodiment, the patient has a known age. In an embodiment, the patient has a known PSA level.

In an embodiment, step (A)(b) comprises measuring said biological sample exosomal miRNA levels comprising exosomal miRNA level of at least one, at least two, or at least three exosomal miRNA from miR-19a, miR-19b, miR-29a, miR-30c, miR-34a, miR-124a, miR-133a, miR-133b, miR-151-3p, miR-191, miR-331, miR-657, miR-664a and miR-875-3p. In another embodiment, step (A)(b) comprises measuring said biological sample exosomal miRNA levels comprising exosomal miRNA level of at least three serum exosomal miRNA selected from miR-29a, miR-34a, miR-331, miR-664a, and miR-875-3p, and/or at least three urine exosomal miRNA selected from miR-26b, miR-29c, miR-99a, miR-195, miR-331, miR-374-5p, miR-454, and miR-590-5p. In another embodiment, step (A)(b) comprise measuring said biological sample exosomal miRNA levels comprising exosomal miRNA level of at least three serum exosomal miRNA selected from miR-19b, miR-34a, miR-664a, and miR-875-3p, and/or at least three urine exosomal miRNA selected from miR-34a, miR-99a, miR-133b, miR-331, miR-374-5p, and miR-454. In another embodiment, step (A)(b) comprises measuring said biological sample exosomal miRNA levels comprising exosomal miRNA level of miR-19a, miR-19b, miR-29a, miR-30c, miR-34a, miR-124a, miR-133a, miR-133b, miR-151-3p, miR-191, miR-331, miR-657, miR-664a and miR-875-3p. In another embodiment, step (A)(b) comprises measuring said biological sample exosomal miRNA levels comprising exosomal miRNA level of serum exosomal miRNA miR-29a, miR-34a, miR-331, miR-664a, and miR-875-3p. In another embodiment, step (A)(b) comprises measuring said biological sample exosomal miRNA levels comprising exosomal miRNA level of urine exosomal miRNA from miR-26b, miR-29c, miR-99a, miR-195, miR-331, miR-374-5p, miR-454, and miR-590-5p. In another embodiment, step (A)(b) comprises measuring said biological sample exosomal miRNA levels comprising exosomal miRNA level of serum exosomal miRNA miR-29a, miR-34a, miR-331, miR-664a, and miR-875-3p, and urine exosomal miRNA from miR-26b, miR-29c, miR-99a, miR-195, miR-331, miR-374-5p, miR-454, and miR-590-5p. In another embodiment, step (A)(b) comprise measuring said biological sample exosomal miRNA levels comprising exosomal miRNA level of serum exosomal miRNA miR-19b, miR-34a, miR-664a, and miR-875-3p, and/or urine exosomal miRNA selected from miR-34a, miR-99a, miR-133b, miR-331, miR-374-5p, and miR-454. In another embodiment, step (A)(b) comprise measuring said biological sample exosomal miRNA levels comprising exosomal miRNA level of serum exosomal miRNA miR-19b, miR-34a, miR-664a, and miR-875-3p. In another embodiment, step (A)(b) comprise measuring said biological sample exosomal miRNA levels comprising exosomal miRNA level of urine exosomal miRNA miR-34a, miR-99a, miR-133b, miR-331, miR-374-5p, and miR-454. In another embodiment, step (A)(b) comprise measuring said biological sample exosomal miRNA levels comprising exosomal miRNA level of serum exosomal miRNA miR-19b, miR-34a, miR-664a, and miR-875-3p, and/or urine exosomal miRNA miR-34a, miR-99a, miR-133b, miR-331, miR-374-5p, and miR-454.

In an embodiment, the biological sample exosomal miRNA profile and the one or more control profiles in (A)(c) and (d) comprise:
(a) the levels of at least two serum exosomal miRNAs selected from miR-29a, miR-30c, miR-151-3p, miR-191 and miR-664a, or at least two urine exosomal miRNAs selected from miR-19a, miR-195, miR-331, miR-374-5p and miR-590-5p;
(b) the levels of at least three serum exosomal miRNAs selected from miR-19a, miR-29a, miR-151-3p and miR-664a; or at least three urine exosomal miRNAs selected from miR-19a, miR-26b, miR-331, miR-374-5p and miR-590-5p; and/or
(c) the levels of at least two serum exosomal miRNAs selected from miR-29a, miR-133a, miR-151-3p and miR-657; or at least one serum exosomal miRNA selected from miR-29a, miR-133a, miR-151-3p and miR-657 and at least one urine exosomal miRNA selected from miR-19a, miR-26a, miR-331 and miR-590.

In an embodiment, the biological sample exosomal miRNA profile comprises the levels of at least two serum exosomal miRNAs selected from miR-29a, miR-30c, miR-151-3p, miR-191 and miR-664a. In another embodiment, the biological sample exosomal miRNA profile comprises the levels of at least urine exosomal miRNAs selected from miR-19a, miR-195, miR-331, miR-374-5p and miR-590-5p. In another embodiment, the biological sample exosomal miRNA profile comprises the levels of at least three serum exosomal miRNAs selected from miR-19a, miR-29a, miR-151-3p and miR-664a. In another embodiment, the biological sample exosomal miRNA profile comprises the levels of at least three urine exosomal miRNAs selected from miR-19a, miR-26b, miR-331, miR-374-5p and miR-590-5p. In another embodiment, the biological sample exosomal miRNA profile comprises the levels of at least two serum exosomal miRNAs selected from miR-29a, miR-133a, miR-151-3p and miR-657. In another embodiment, the biological sample exosomal miRNA profile comprises the levels of at least one serum exosomal miRNA selected from miR-29a, miR-133a, miR-151-3p and miR-657 and at least one urine exosomal miRNA selected from miR-19a, miR-26a, miR-331 and miR-590. In another embodiment, the biological sample exosomal miRNA profile comprises the levels of serum exosomal miRNAs miR-29a, miR-30c, miR-151-3p, miR-191 and miR-664a. In another embodiment, the biological sample exosomal miRNA profile comprises the levels of urine exosomal miRNAs miR-19a, miR-195, miR-331, miR-374-5p and miR-590-5p. In another embodiment, the biological sample exosomal miRNA profile comprises the levels of serum exosomal miRNAs miR-19a, miR-29a, miR-151-3p and miR-664a. In another embodiment, the biological sample exosomal miRNA profile comprises the levels of urine exosomal miRNAs miR-19a, miR-26b, miR-331, miR-374-5p and miR-590-5p. In another embodiment, the biological sample exosomal miRNA profile comprises the levels of serum exosomal miRNAs miR-29a, miR-133a, miR-151-3p and miR-657. In another embodiment, the biological sample exosomal miRNA profile comprises the levels of serum exosomal miRNA miR-29a, miR-133a, miR-151-3p and miR-657 and urine exosomal miRNA miR-19a, miR-26a, miR-331 and miR-590. In another embodiment, the biological sample exosomal miRNA profile and the one or more control profiles comprise (i) the levels of at least three serum exosomal miRNAs selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p, and/or (ii) at least three urine exosomal miRNA selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p.

Also provided is a method for selecting a treatment for prostate cancer in a patient preoperative of prostatectomy with known age and/or PSA level, comprising:
(a) providing at least one biological sample from a patient, wherein the biological sample is serum or urine;
(b) measuring exosomal miRNA levels of said biological sample;
(c) determining exosomal miRNA profile of said biological sample;
(d) determining or measuring the level of similarity of said biological sample exosomal miRNA profile to one or more control profiles, wherein (i) a low level of similarity of the sample profile to an active surveillance control profile; (ii) a high level of similarity to a prostatectomy control profile; and/or (iii) a lower level of similarity to an active surveillance control profile than to a prostatectomy control profile indicates high disease aggressiveness; or wherein (iv) a high level of similarity of the sample exosomal miRNA profile to an active surveillance control profile; (v) a low level of similarity to a prostatectomy control profile; and/or (vi) a higher level of similarity to an active surveillance control profile than to a prostatectomy control profile indicates low disease aggressiveness; and
(e) selecting said patient for prostatectomy when said patient has high disease aggressiveness; or selecting said patient for monitoring prostate cancer progression annually when said patient has low disease aggressiveness.

In an embodiment, the patient has a known age and known PSA level. In an embodiment, the patient has a known age. In an embodiment, the patient has a known PSA level.

In an embodiment, the biological sample exosomal miRNA profile and the one or more control profiles in (c) and (d) comprise:
(a) the levels of at least two serum exosomal miRNAs selected from miR-29a, miR-30c, miR-151-3p, miR-191 and miR-664a, or at least two urine exosomal miRNAs selected from miR-19a, miR-195, miR-331, miR-374-5p and miR-590-5p;
(b) the levels of at least three serum exosomal miRNAs selected from miR-19a, miR-29a, miR-151-3p and miR-664a; or at least three urine exosomal miRNAs selected from miR-19a, miR-26b, miR-331, miR-374-5p and miR-590-5p; and/or
(c) the levels of at least two serum exosomal miRNAs selected from miR-29a, miR-133a, miR-151-3p and miR-657; or at least one serum exosomal miRNA selected from miR-29a, miR-133a, miR-151-3p and miR-657 and at least one urine exosomal miRNA selected from miR-19a, miR-26a, miR-331 and miR-590.

In an embodiment, the biological sample exosomal miRNA profile comprises the levels of at least two serum exosomal miRNAs selected from miR-29a, miR-30c, miR-151-3p, miR-191 and miR-664a. In another embodiment, the biological sample exosomal miRNA profile comprises the levels of at least urine exosomal miRNAs selected from miR-19a, miR-195, miR-331, miR-374-5p and miR-590-5p. In another embodiment, the biological sample exosomal miRNA profile comprises the levels of at least three serum exosomal miRNAs selected from miR-19a, miR-29a, miR-151-3p and miR-664a. In another embodiment, the biological sample exosomal miRNA profile comprises the levels of at least three urine exosomal miRNAs selected from miR-19a, miR-26b, miR-331, miR-374-5p and miR-590-5p. In another embodiment, the biological sample exosomal miRNA profile comprises the levels of at least two serum exosomal miRNAs selected from miR-29a, miR-133a, miR-151-3p and miR-657. In another embodiment, the biological sample exosomal miRNA profile comprises the levels of at least one serum exosomal miRNA selected from miR-29a, miR-133a, miR-151-3p and miR-657 and at least one urine exosomal miRNA selected from miR-19a, miR-26a, miR-331 and miR-590. In another embodiment, the biological sample exosomal miRNA profile comprises the levels of serum exosomal miRNAs miR-29a, miR-30c, miR-151-3p, miR-191 and miR-664a. In another embodiment, the biological sample exosomal miRNA profile comprises the levels of urine exosomal miRNAs miR-19a, miR-195, miR-331, miR-374-5p and miR-590-5p. In another embodiment, the biological sample exosomal miRNA profile comprises the levels of serum exosomal miRNAs miR-19a, miR-29a, miR-151-3p and miR-664a. In another embodiment, the biological sample exosomal miRNA profile comprises the levels of urine exosomal miRNAs miR-19a, miR-26b, miR-331, miR-374-5p and miR-590-5p. In another embodiment, the biological sample exosomal miRNA profile comprises the levels of serum exosomal miRNAs miR-29a, miR-133a, miR-151-3p and miR-657. In another embodiment, the biological sample exosomal miRNA profile comprises the levels of serum exosomal miRNA miR-29a, miR-133a, miR-151-3p and miR-657 and urine exosomal miRNA miR-19a, miR-26a, miR-331 and miR-590. In another embodiment, the biological sample exosomal miRNA profile and the one or more control profiles comprise (i) the levels of at least three serum exosomal miRNAs selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p, and/or (ii) at least three urine exosomal miRNA selected from miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p.

The level or amount of exosomal miRNAs in a biological sample may be determined or measured by any suitable method. Any reliable method for measuring the level or amount of exosomal miRNA in a sample may be used. For example, exosomal miRNA can be isolated by various known methods, including the use of kits such as Norgen Urine Exosome RNA Isolation Kit and RNA Clean-up and Concentration Micro Kit (Norgen biotek, Thorold, Canada), and Qiagen exoRNeasy Serum/Plasma Midi Kit (Qiagen, Hilden, Germany). Exosomal miRNA can be detected and quantified by amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays such as microarrays), NanoStringm analysis, Northern Blot analysis, branched DNA (bDNA) signal amplification, and in situ hybridization), and sequencing-based methods (e.g. next-generation sequencing methods, for example, using the Illumina or IonTorrent platforms). Other exemplary techniques include ribonuclease protection assay (RPA) and mass spectroscopy. However, some of these techniques, such as most variations of PCR, are known to introduce quantification bias during amplification.

One preferred technique as presently disclosed involves the highly specific and sensitive Droplet Digital PCR (ddPCR). Digital PCR takes advantage of nucleic acid amplification on a single molecule level, and offers a highly sensitive method for quantifying low copy number nucleic acid. Fluidigm® Corporation, BioRad's Digital PCR and Raindance technologies all offer systems for the digital analysis of nucleic acids. ddPCR technology has unprecedented sensitivity in addition to the ability for absolute quantification, resulting in great reproducibility. Moreover, ddPCR overcomes most PCR problems, including the bias introduced during the pre-amplification. In addition, other methods that are similarly sensitive, accurate and highly reproducible, such as digital molecular barcoding (e.g. NanoString's nCounter technology) and next-generation sequencing (i.e. High-throughput sequencing), are also useful in measuring and determining levels or amounts of exosomal miRNAs in a biological sample. In an embodiment, the determining or measuring of exosomal miRNA levels involves using ddPCR, digital molecular barcoding, or next-generation sequencing.

Thus, the methods described herein involve monitoring prostate cancer progression for predicting, or identifying patients that are at low risk of disease progression, having indolent disease or low disease aggressiveness. Present methods select active surveillance for said patients having low risk of disease progression, having indolent disease or low disease aggressiveness. As well, the methods described herein also select prostatectomy for said patients having high risk of disease progression, high disease aggressiveness or predicted to have biochemical failure. In an embodiment, the methods described herein involve selecting a patient for active surveillance when said patient has low risk of disease progression, low disease aggressiveness, or indolent disease. In another embodiment, the methods described herein involve selecting a patient for prostatectomy when said patient has high risk of disease progression, high disease aggressiveness, or predicted to have biochemical failure.

The present disclosure also provides a kit for analyzing serum or urine sample for monitoring prostate cancer progression in a patient. In an embodiment, a kit for analyzing serum or urine sample to monitor prostate cancer progression in a patient comprising:
a probe that detects the presence of exosomal miRNA, and
instructions for use,
wherein the patient is preoperative of prostatectomy with a known age and/or PSA level.

In an embodiment of a method disclosed herein, a biological sample exosomal miRNA profile comprises serum exosomal miRNA level of miR-29a. In an embodiment of a method disclosed herein, a biological sample exosomal miRNA profile comprises serum exosomal miRNA level of miR-590-5. In an embodiment of a method disclosed herein, a biological sample exosomal miRNA profile comprises serum exosomal miRNA level of miR-29a and miR-30c. In an embodiment of a method disclosed herein, a biological sample exosomal miRNA profile comprises serum exosomal miRNA level of miR29a and miR-133a. In an embodiment of a method disclosed herein, a biological sample exosomal miRNA profile comprises serum exosomal miRNA level of miR29a and miR-151-3p. In an embodiment of a method disclosed herein, a biological sample exosomal miRNA profile comprises serum exosomal miRNA level of miR-29a and miR-191. In an embodiment of a method disclosed herein, a biological sample exosomal miRNA profile comprises serum exosomal miRNA level of miR29a and miR-657. In an embodiment of a method disclosed herein, a biological sample exosomal miRNA profile comprises serum exosomal miRNA level of miR-29a and miR-664a. In an embodiment of a method disclosed herein, a biological sample exosomal miRNA profile comprises serum exosomal miRNA level of miR-30c and miR-151-3p. In an embodiment of a method disclosed herein, a biological sample exosomal miRNA profile comprises serum exosomal miRNA level of miR-30c and miR-191. In an embodiment of a method disclosed herein, a biological sample exosomal miRNA profile comprises serum exosomal miRNA level of miR-30c and miR-664a. In an embodiment of a method disclosed herein, a biological sample exosomal miRNA profile comprises serum exosomal miRNA level of miR-133a and miR-151-3p. In an embodiment of a method disclosed herein, a biological sample exosomal miRNA profile comprises serum exosomal miRNA level of miR-133a and miR-657. In an embodiment of a method disclosed herein, a biological sample exosomal miRNA profile comprises serum exosomal miRNA level of miR-151-3p and miR-191. In an embodiment of a method disclosed herein, a biological sample exosomal miRNA profile comprises serum exosomal miRNA level of miR-151-3p and miR-657. In an embodiment of a method disclosed herein, a biological sample exosomal miRNA profile comprises serum exosomal miRNA level of miR-151-3p and miR-664a. In an embodiment of a method disclosed herein, a biological sample exosomal miRNA profile comprises serum exosomal miRNA level of miR-191 and miR-664a. In an embodiment of a method disclosed herein, a biological sample exosomal miRNA profile comprises serum exosomal miRNA level of miR-19a, miR-29a and miR-151-3p. In an embodiment of a method disclosed herein, a biological sample exosomal miRNA profile comprises serum exosomal miRNA level of miR-19a, miR-29a and miR-664a. In an embodiment of a method disclosed herein, a biological sample exosomal miRNA profile comprises serum exosomal miRNA level of miR-19a, miR-151-3p and miR-664a. In an embodiment of a method disclosed herein, a biological sample exosomal miRNA profile comprises serum exosomal miRNA level of miR-29a, miR-133a and miR-151-3p. In an embodiment of a method disclosed herein, a biological sample exosomal miRNA profile comprises serum exosomal miRNA level of miR-29a, miR-133a and miR-664a. In an embodiment of a method disclosed herein, a biological sample exosomal miRNA profile comprises serum exosomal miRNA level of miR-29a, miR-151-3p and miR-664a. In an embodiment of a method disclosed herein, a biological sample exosomal miRNA profile comprises serum exosomal miRNA level of miR-151-3p and miR-657. In an embodiment of a method disclosed herein, a biological sample exosomal miRNA profile comprises serum exosomal miRNA level of miR-29a, miR-151-3p and miR-664a. In an embodiment of a method disclosed herein, a biological sample exosomal miRNA profile comprises serum exosomal miRNA level of miR-19a, miR-29a, miR-151-3p and miR-664a. In an embodiment of a method disclosed herein, a biological sample exosomal miRNA profile comprises serum exosomal miRNA level of miR-19a, miR-133a, miR-151-3p and miR-657. In an embodiment of a method disclosed herein, a biological sample exosomal miRNA profile comprises serum exosomal miRNA level of miR-29a, miR-133a, miR-151-3p and miR-657. In an embodiment of a method disclosed herein, a biological sample exosomal miRNA profile comprises serum exosomal miRNA level of miR-19b, miR-34a, miR-664a and miR-875-3p. In an embodiment of a method disclosed herein, a biological sample serum exosomal miRNA profile comprises exosomal miRNA level of miR-29a, miR-30c, miR-151-3p, miR-191 and miR-664a. In an embodiment of a method disclosed herein, a biological sample exosomal miRNA profile comprises serum exosomal miRNA level of miR-29a, miR-34a, miR-331, miR-664a and miR-875-3p. In an embodiment of a method disclosed herein, a biological sample exosomal miRNA profile comprises urine exosomal miRNA level of miR-331. In an embodiment of a method disclosed herein, a biological sample exosomal miRNA profile comprises urine exosomal miRNA level of miR-19a and miR-195. In an embodiment of a method disclosed herein, a biological sample exosomal miRNA profile comprises urine exosomal miRNA level of miR-19a and miR-331. In an embodiment of a method disclosed herein, a biological sample exosomal miRNA profile comprises urine exosomal miRNA level of miR-19a and miR-374-5p. In an embodiment of a method disclosed herein, a biological sample exosomal miRNA profile comprises urine exosomal miRNA level of miR-19a and miR-590-5p. In an embodiment of a method disclosed herein, a biological sample exosomal miRNA profile comprises urine exosomal miRNA level of miR-99a and miR-374-5p. In an embodiment of a method disclosed herein, a biological sample exosomal miRNA profile comprises urine exosomal miRNA level of miR-195 and miR-331. In an embodiment of a method disclosed herein, a biological sample exosomal miRNA profile comprises urine exosomal miRNA level of miR-195 and miR-374-5p. In an embodiment of a method disclosed herein, a biological sample exosomal miRNA profile comprises urine exosomal miRNA level of miR-195 and miR-590-5p. In an embodiment of a method disclosed herein, a biological sample exosomal miRNA profile comprises urine exosomal miRNA level of miR-331 and miR-374-5p. In an embodiment of a method disclosed herein, a biological sample exosomal miRNA profile comprises urine exosomal miRNA level of miR-331 and miR-590-5p. In an embodiment of a method disclosed herein, a biological sample exosomal miRNA profile comprises urine exosomal miRNA level of miR-374-5p and miR-590-5p. In an embodiment of a method disclosed herein, a biological sample exosomal miRNA profile comprises urine exosomal miRNA level of miR-331, miR-374-5p and 590-5p. In an embodiment of a method disclosed herein, a biological sample exosomal miRNA profile comprises urine exosomal miRNA level of miR-19a, miR-26b and miR-311. In an embodiment of a method disclosed herein, a biological sample exosomal miRNA profile comprises urine exosomal miRNA level of miR-19a, miR-26b and miR-374-5p. In an embodiment of a method disclosed herein, a biological sample exosomal miRNA profile comprises urine exosomal miRNA level of miR-19a, miR-26b and miR-590-5p. In an embodiment of a method disclosed herein, a biological sample exosomal miRNA profile comprises urine exosomal miRNA level of miR-26b, miR-311 and miR-374-5p. In an embodiment of a method disclosed herein, a biological sample exosomal miRNA profile comprises urine exosomal miRNA level of miR-26b, miR-311 and miR-590-5p. In an embodiment of a method disclosed herein, a biological sample exosomal miRNA profile comprises urine exosomal miRNA level of miR-331, miR-374-5p and miR-590-5p. In an embodiment of a method disclosed herein, a biological sample exosomal miRNA profile comprises urine exosomal miRNA level miR-19a, miR-26b, miR-331 and miR-590-5p. In an embodiment of a method disclosed herein, a biological sample exosomal miRNA profile comprises urine exosomal miRNA level of miR-19a, miR-26a, miR-331 and miR-590-5p. In an embodiment of a method disclosed herein, a biological sample exosomal miRNA profile comprises urine exosomal miRNA level of miR-19a, miR-29c, miR-99a and miR-590-5p. In an embodiment of a method disclosed herein, a biological sample exosomal miRNA profile comprises urine exosomal miRNA level of miR-26b, miR-195, miR-374-5p and miR-590-5p. In an embodiment of a method disclosed herein, a biological sample exosomal miRNA profile comprises urine exosomal miRNA level of miR-99a, miR-331, miR-374-5p and miR-590-5p. In an embodiment of a method disclosed herein, a biological sample exosomal miRNA profile comprises urine exosomal miRNA level of miR-195, miR-331, miR-374-5p and miR-590-5p. In an embodiment of a method disclosed herein, a biological sample exosomal miRNA profile comprises urine exosomal miRNA level of miR-19a, miR-26b, miR-331, miR-374-5p and miR-590-5p. In an embodiment of a method disclosed herein, a biological sample exosomal miRNA profile comprises urine exosomal miRNA level of miR-19a, miR-195, miR-331, miR-374-5p and miR-590-5p. In an embodiment of a method disclosed herein, a biological sample exosomal miRNA profile comprises urine exosomal miRNA level of miR-26b, miR-195, miR-331, miR-374-5p and miR-590-5p. In an embodiment of a method disclosed herein, a biological sample exosomal miRNA profile comprises urine exosomal miRNA level of miR-34a, miR-99a, miR-133b, miR-331, miR-374-5p and miR-454. In an embodiment of a method disclosed herein, a biological sample exosomal miRNA profile comprises urine exosomal miRNA level of miR-26b, miR-29c, miR-99a, miR-195, miR-331, miR-374-5p, miR-454 and miR-590-5p. In an embodiment of a method disclosed herein, a biological sample exosomal miRNA profile comprises urine exosomal miRNA level of miR-19a, miR-26b, miR-29c, miR-99a, miR-191, miR-195, miR-331, miR-374-5p, miR-378, miR-454 and miR-590-5p. In an embodiment of a method disclosed herein, a biological sample exosomal miRNA profile comprises serum exosomal miRNA level of miR-29a and urine exosomal miRNA level of miR-26b. In an embodiment of a method disclosed herein, a biological sample exosomal miRNA profile comprises miRNA level of at least one serum exosomal miRNA selected from miR-29a, miR-133a, miR-657 and miR-151-3p, and at least one miRNA level of at least one urine exosomal miRNA selected from of miR-19a, miR-26a, miR-331 and miR-590. In an embodiment of a method disclosed herein, the patient has a known age and known PSA level. In an embodiment of a method disclosed herein, the patient has a known age. In an embodiment of a method disclosed herein, the patient has a known PSA level. In an embodiment of a method disclosed herein, the level of miRNA is normalized by the level of miR-29a. In any of the embodiments provided herein, the level of miRNA is normalized by the level of creatinine.

The probe is a set of miRNA-specific primers, suitable for techniques such as qPCR, optionally ddPCR. Specifically, the probe of the present disclosure targets the miRNAs described herein. In an embodiment, the probe is a set of exosomal miRNA-specific primers.

In another embodiment, the set of exosomal miRNA-specific primers comprises primers targeting miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a or miR-875-3p. In another embodiment, the set of exosomal miRNA-specific primers comprises primers targeting at least three of miR-19a, miR-19b, miR-26b, miR-29a, miR-29c, miR-30c, miR-34a, miR-99a, miR-124a, miR-133a, miR-133b, miR-151-3p, miR-191, miR-195, miR-331, miR-365, miR-374-5p, miR-378, miR-454, miR-590-5p, miR-657, miR-664a, and miR-875-3p.

The kits described herein contain combinations of primer sets. In an embodiment, the set of exosomal miRNA-specific primers comprises primers targeting at least two of miR-29a, miR-30c, miR-151-3p, miR-191 and miR-664a, or at least two of miR-19a, miR-195, miR-331, miR-374-5p and miR-590-5p; the set of exosomal miRNA-specific primers comprises primers targeting at least three of miR-19a, miR-29a, miR-151-3p and miR-664a, or at least three of miR-19a, miR-26b, miR-331, miR-374-5p and miR-590-5p; and at least two of miR-29a, miR-133a, miR-151-3p and miR-657; or the set of exosomal miRNA-specific primers comprises primers targeting at least one of miR-29a, miR-133a, miR-151-3p and miR-657 and at least one of miR-19a, miR-26a, miR-331 and miR-590.

In another embodiment, the set of exosomal miRNA-specific primers comprises primers targeting at least two of miR-29a, miR-30c, miR-151-3p, miR-191 and miR-664a, or at least two of miR-19a, miR-195, miR-331, miR-374-5p and miR-590-5p.

In another embodiment, the set of exosomal miRNA-specific primers comprises primers targeting at least three of miR-19a, miR-29a, miR-151-3p and miR-664a, or at least three of miR-19a, miR-26b, miR-331, miR-374-5p and miR-590-5p.

In another embodiment, the set of exosomal miRNA-specific primers comprises primers targeting at least two of miR-29a, miR-133a, miR-151-3p and miR-657; or the set of exosomal miRNA-specific primers comprises primers targeting at least one of miR-29a, miR-133a, miR-151-3p and miR-657 and at least one of miR-19a, miR-26a, miR-331 and miR-590.

In another embodiment, the set of exosomal miRNA-specific primers comprises primers targeting at least three of miR-29a, miR-34a, miR-331, miR-664a, and miR-875-3p; the set of exosomal miRNA-specific primers comprises primers targeting at least three of miR-26b, miR-29c, miR-99a, miR-195, miR-331, miR-374-5p, miR-454, and miR-590-5p; the set of exosomal miRNA-specific primers comprises primers targeting at least three of miR-19b, miR-34a, miR-664a, and miR-875-3p; or the set of exosomal miRNA-specific primers comprises primers targeting at least three of miR-34a, miR-99a, miR-133b, miR-331, miR-374-5p, and miR-454.

The following non-limiting Examples are illustrative of the present disclosure:

### Example

Identification of Exosomal Serum and Urinary miRNA Biomarker Panel to Guide Active Surveillance in Prostate Cancer

In the field of biomarker discovery, liquid biopsy has many advantages, including being non-invasive, less expensive and easier to access, so it can be repeated more frequently⁶. In the era of precision medicine, the promise is to divide patients into biologically distinct groups based on tumor biology rather than relying solely on tumor morphology.

miRNAs are small noncoding RNA molecules (20-25 nucleotides in length)^{3, 5}. Recent studies have shown that miRNAs are promising biomarkers for prostate and other cancers⁹. miRNAs represent attractive biomarker class because they are stable, can be accurately quantified and are actively secreted as exosomal miRNAs in a number of biological fluids including serum and urine¹⁰.

The present Example describes identification of exosomal serum and urinary miRNA biomarker panel to guide active surveillance or prostatectomy in prostate cancer.

### Materials and Methods

### Patient Cohort

This study included 462 cases diagnosed with prostate cancer at University Health Network (UHN), Toronto, Canada. The study was approved by the research ethics boards of St. Michael's Hospital and UHN. All patients underwent prostatectomy. Preoperative urine and serum were collected. Urine samples were centrifuged at 4° C., aliquoted and cryopreserved in −80° C. Serum separator tubes were used to collect serum and centrifuged at 4° C. The resulting supernatant was collected, aliquoted and cryopreserved.

Demographic and clinical data were obtained through interview and Genitourinary Biobank medical record database. All patients gave informed consent to provide specimens for research study. Patient demographics and clinical data are summarized in Table 1B.

TABLE 1B
Clinical parameters of the patient cohort.

| | | | | | Biochemical failure | | | | Tumor volume | |
|---|---|---|---|---|---|---|---|---|---|---|
| Gleason | Number of | Percentage | Age | | Yes | | No | | (%) | |
| score | patients | (%) | Mean | SD | [n, (%)] | | [(n, %)] | | Mean | SD |
| G5 | 7 | 1.56 | 61.29 | 2.81 | 0 | (0) | 7 | (100) | 4.57 | 3.26 |
| G6 | 206 | 43.88 | 58.36 | 7.10 | 8 | (3.88) | 189 | (91.75) | 5.54 | 7.47 |
| G7 | 224 | 49.67 | 62.52 | 6.21 | 72 | (32.14) | 151 | (67.41) | 9.10 | 10.82 |
| G8 | 7 | 1.56 | 61.14 | 9.04 | 7 | (100) | 0 | (0) | 14.86 | 12.88 |
| G9 | 15 | 3.34 | 62.67 | 6.96 | 15 | (100) | 0 | (0) | 21.57 | 23.40 |

| | | | | | | | | |
|---|---|---|---|---|---|---|---|---|
| Total | 459 | 100 | 60.66 | 6.96 | 102 | 347 | 7.88 | 10.53 |

Urine and Serum Exosomal miRNA Isolation

Total exosomal miRNAs were isolated from 1 ml urine and concentrated using Norgen Urine Exosome RNA Isolation Kit and RNA Clean-up and Concentration Micro Kit (Norgen biotek, Thorold, Canada). Serum exosomal miRNAs were extracted using Qiagen exoRNeasy Serum/Plasma Midi Kit (Qiagen, Hilden, Germany). Each of these methods was performed according to manufacturer's instructions. In total, 10 μl of urinal exosomal miRNA and 14 μl of serum exosomal miRNAs were obtained for further analysis.

Reverse Transcription and Droplet Digital PCR (ddPCR)

Exosomal miRNA expression levels were assessed using the Taqman microRNA assays (ThermoFisher). Exosomal miRNA-specific primers were pooled for RT using TaqMan® MicroRNA Reverse Transcription kits (Life Technologies, California, USA). According to the protocol, candidate miRNAs RT Primers were dissolved in 1×TE buffer and pooled. ddPCR was conducted using Taqman probe with FAM or VIC. Optimized PCR conditions were identified by comparing product yields at different annealing temperature, cycling numbers and assay concentrations. Sequence information and accession numbers of miRNA disclosed herein are shown in Table 1A.

Four μl of RNA from each sample was used for reverse transcription (RT) for a total reaction volume of 15 μl. A reaction mixture of 20 μl comprising Bio-Rad ddPCR™ Super Mix for Probes (no UTP) (Bio-Rad, California, USA), exosomal miRNA assay probe and DNA was dispensed into a well of a 96-well plate. Droplets were generated by QX200 Automatic Droplet Generator (Bio-Rad). The PCR reaction was carried out in a Bio-Rad C1000 Touch Thermal Cycler (Bio-Rad Laboratories Inc.) with a 96-deep well reaction module using the following program: (1) 95° C. for 5 minutes, (2) 94° C. for 30 seconds, (3) 60° C. for 90 seconds, (4) steps 2 and 3 repeated for 45 cycles, (5) 98° C. for 10 minutes, and (6) an infinite hold at 4° C. In between each step of the protocol, the ramp rate was 2° C./second to ensure the droplet temperature changes in conjunction with the surrounding oil.

After thermal cycling, the plate was placed in the block of a Bio-Rad QX200 Droplet Reader (Bio-Rad Laboratories Inc.). Droplets were read at a rate of 32 wells/hour and data were analyzed in QuantaSoft version 1.7.4 (Bio-Rad Laboratories Inc.). Single well threshold was used to group droplets using the software's default internal algorithm set, if necessary, set threshold or clusters manually. Poisson statistics were also determined by the software. Droplets which were more than 10,000 will be considered as valid events and counted the detecting exosomal miRNAs copy number.

### Statistics Analysis

Statistical analysis was performed by using IBM SPSS Statistics for Windows, version 20.0 (IBM Corp., Armonk, N.Y., USA). Receiver Operating Characteristic (ROC) curves were generated using and cut-offs were determined to provide maximum sensitivity, specificity or combination of both to evaluate the ability of the chosen exosomal miRNAs or the combined exosomal miRNA models to discriminate patients in terms of Gleason Grade and tumor volume. Kaplan-Meier curves together with Log-rank tests were used to assess exosomal miRNA values in predicting biochemical failure. Cox regression analysis (multiple parameters analysis) was used to determine the prognostic biomarkers for prostate cancer biochemical failure.

Cox proportional hazards regression model and multivariable logistic regression analyses comparing miRNAs and other clinical parameters with relapse-free survival were performed in order to distinguish low-from high-risk prostate cancer (defined as having Gleason Grade Group 3+4+5 assessed in the resected prostate and the occurrence of biochemical failure following treatment). The relationship of each risk factor was compared with outcome in the form of study endpoints. To adjust for any inflated false positive results due to multiple comparisons, this single risk factor analysis was adjusted by false discovery rate (FDR). A 10-fold cross validation to identify the optimal predictive model and internally validate the model performance. A predictive risk score (PRS) was determined using the weighted combination of the risk factors in the selected model. The predictive accuracy and performance characteristics of the model were assessed by using the concordance statistic (C-statistic, also known as c-index) and the area under the curve (AUC) of the receiver operating characteristics and calibration plots. Performance of the model was compared with published risk assessment models.

### Results

Descriptive statistics are shown in Table 1B. The mean age of patients in this study was 60.66 years. Preoperative PSA value, prostatectomy (rather than biopsy) Gleason grade score and tumor volume (involvement percentage) were collected for each patient and used for statistical analysis. Within the cohort, the Gleason scores of 213 patients were less than 6/10 and the Gleason scores of 246 patients were greater than 7/10. 102 patients had biochemical failure, and 347 did not develop biochemical failure. The mean tumor volume was 7.88%.

Based on published reports^{1, 9, 11, 15, 16, 19, 25} dysregulated miRNAs were selected for assessment of their clinical utility as biomarkers in serum (14 miRNAs) and urine (16 miRNAs). Absolute quantification of exosomal miRNAs in serum and urine was determined by droplet digital PCR (ddPCR). Exosomal miRNA expression was determined as a categorical variable (positive vs. negative) based on a statistically determined cut-off value, as shown in Table 2A.

TABLE 2A
Cut-off values for exosomal serum and urine miRNAs.

| | | | Cut-off | Cut-off |
|---|---|---|---|---|
| | | | (normalized by | (normalized |
| | Cut-off value | | creatinine) | by miR-29a) |
| Serum | (Copies/ml | Urine | (Copies/ml | (Copies/ml |
| miRNAs | serum) | miRNAs | urine) | urine) |
| miR-124a | 21175 | miR-133b | 265.38 | 720.83 |
| miR-133a | 1010.625 | miR-191 | 134.74 | 455.61 |
| miR-133b | 9143.75 | miR-195 | 1979.52 | 1196.73 |
| miR-151-3p | 279.125 | miR-19a | 11603.34 | 7596.31 |
| miR-191 | 96.25 | miR-26b | 26056.96 | 20553.21 |
| miR-19a | 13475 | miR-29a | 7119.82 | — |
| miR-19b | 115.5 | miR-29c | 11262.27 | 9618.93 |
| miR-29a | 3465 | miR-331 | 3468.31 | 3295.48 |
| miR-30c | 462 | miR-34a | 694.2 | 850.41 |
| miR-331 | 1270.5 | miR-365 | 2226.71 | 1832.9 |
| miR-34a | 5005 | miR-374-5p | 6609.72 | 6243.86 |
| miR-657 | 16362.5 | miR-378 | 346.83 | 923.79 |
| miR-664a | 327.25 | miR-454 | 1881.54 | 2462.11 |
| miR-875-3p | 288.75 | miR-590-5p | 1243.28 | 1076.95 |
| | | miR-875-3p | 136.95 | 140.93 |
| | | miR-99a | 880.61 | 855.62 |

Preoperative Serum Exosomal miRNAs can Predict Disease Relapse

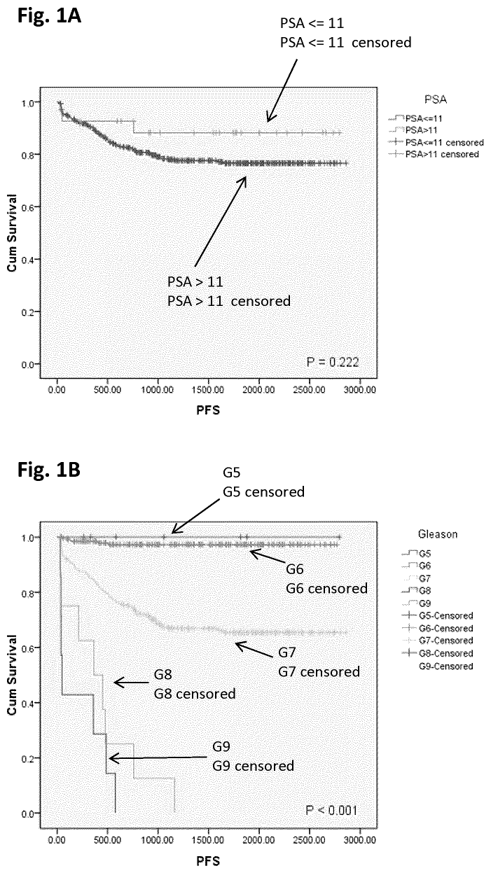

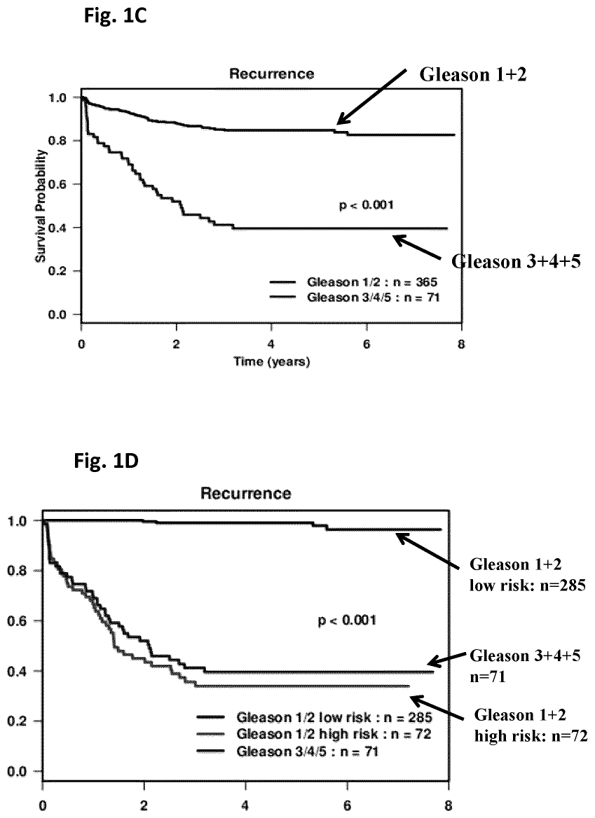

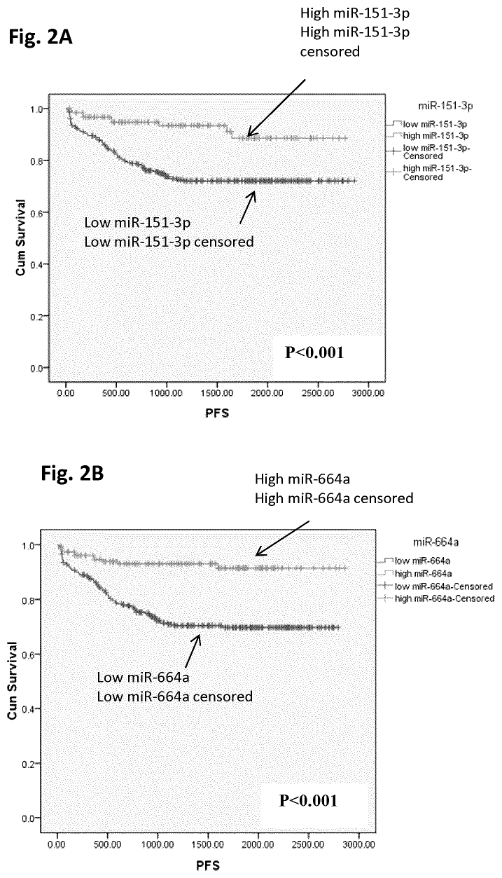

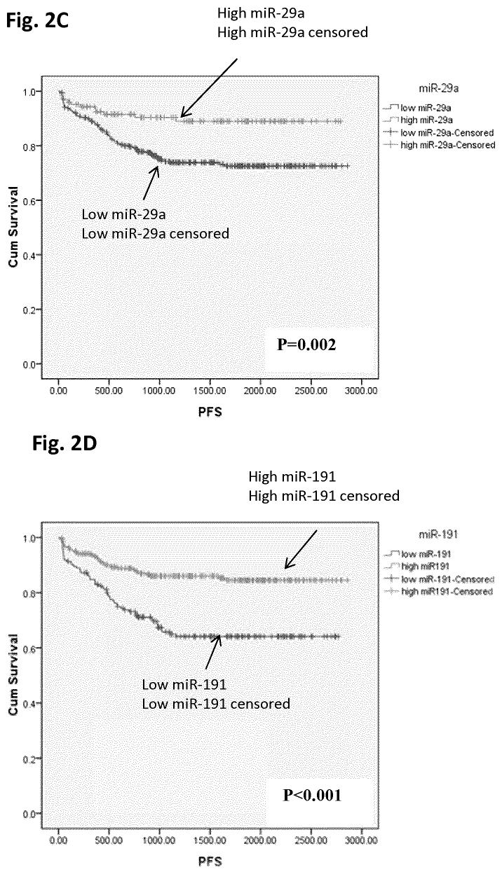

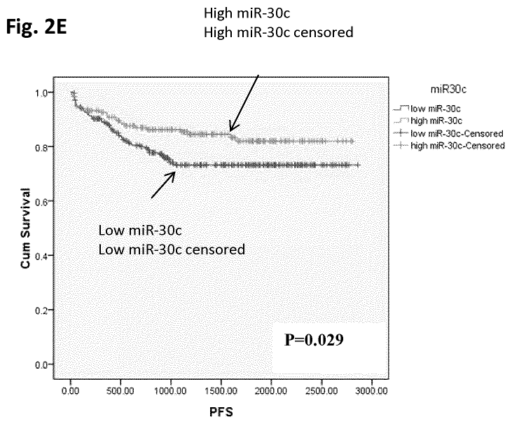

Gleason score has limited clinical utility as a preoperative biomarker to guide the treatment of surgery vs. active surveillance because accurate guidance is based on data from prostatectomy rather than biopsy evaluation of the Gleason score. In the patient cohort, preoperative serum PSA alone was not able to predict the development of biochemical failure (disease relapse) after prostatectomy, as expected (p=0.222, FIG. 1A). High Gleason score, only along with data from prostatectomy, was able to predict biochemical failure and segregate the cohort into distinct prognostic categories (p<0.001, FIG. 1B). By further combining Gleason subgroups 1+2 and 3+4+5, a distinct separation in recurrence is observed (FIG. 1C). Table 2B shows the number of subjects available for analysis in Gleason Grade Group 1+2 and Gleason Grade Group 3+4+5 between each interval in FIG. 1C. Further stratification can be determined within Gleason subgroup 1+2 to identify patients at high risk of biochemical failure (FIG. 1D). Table 2C shows the number of subjects available for analysis in each group between each interval in FIG. 1D. In comparison, five exosomal miRNAs were identified for which preoperative serum levels were able to accurately predict prostate cancer relapse (FIG. 2). These are miR-151-3p (p<0.001) miR-664a (p<0.001), miR-29a (p=0.002), miR-191 (p<0.001) and miR-30c (p=0.029). Moreover, multivariate analysis (Cox regression analysis) indicated that miR-29a and miR-664a are independent markers for disease relapse along with operative tumor volume and prostatectomy Gleason score (Table 3).

TABLE 2B
Values represent the number of subjects available for analysis
in two groups (Gleason Grade Group 1 + 2 and Gleason
Grade Group 3 + 4 + 5) between each interval in FIG. 1C.

| | Interval: | | | |
|---|---|---|---|---|
| | 0 to 2 years | 2 to 4 years | 4 to 6 years | 6 to 8 years |
| Gleason Grade | 365 | 265 | 161 | 58 |
| Group 1 + 2 | | | | |
| Gleason Grade | 71 | 35 | 21 | 5 |
| Group | | | | |
| 3 + 4 + 5 | | | | |

TABLE 2C
Values represent the number of subjects available for analysis
under each group between each interval in FIG. 1D.

Interval:

| | 0 to 2 | 2 to 4 | 4 to 6 | 6 to 8 |
|---|---|---|---|---|
| | years | years | years | years |
| Gleason Grade Group | 285 | 228 | 141 | 51 |
| 1 + 2 low risk | | | | |
| Gleason Grade Group | 72 | 30 | 15 | 5 |
| 3 + 4 + 5 | | | | |
| Gleason Grade Group | 71 | 35 | 21 | 5 |
| 1 + 2 high risk | | | | |

TABLE 3
Cox regression analysis of serum exosomal miRNAs.

95.0% CI

| Serum Variables | p | HR | Lower | Upper |
|---|---|---|---|---|
| Prostatectomy | <0.001* | 20.52 | 7.28 | 57.86 |
| Gleason score | | | | |
| Tumor Volume | <0.001* | 2.21 | 1.30 | 3.77 |
| miR-29a | <0.001* | 3.33 | 1.56 | 7.10 |
| miR-664a | 0.05* | 0.50 | 0.25 | 1.01 |
| miR-124a | 0.87 | 0.96 | 0.58 | 1.58 |
| miR-151-3p | 0.62 | 0.82 | 0.38 | 1.76 |
| miR-191 | 0.10 | 0.68 | 0.44 | 1.07 |

HR = hazard ratio;
*statistically significant value

Serum Exosomal miRNAs Predict Survival in Specific Gleason Grade Groups

A newly published grade grouping system was used in analysis of the cohort. This system groups different scores into a 5-tier system with prognostic significance. It is now adopted by the International Society of Urologic Pathology (ISUP)^{8, 17} (Table 4).

TABLE 4
The updated Gleason Grade Grouping system.

| | Gleason Grade Group |
|---|---|
| Gleason score | pattern |
| 3 + 3 | Grade Group 1 |
| 3 + 4 | Grade Group 2 |
| 4 + 3 | Grade Group 3 |
| 4 + 4 or 3 + 5 or 5 + 3 | Grade Group 4 |
| >8 | Grade Group 5 |

As shown in Table 5A, three exosomal miRNAs were identified as having expression levels that can stratify specific Gleason Grade Group grades into distinct prognostic groups and suggests each of these groups is a heterogeneous population with patients who will develop biochemical failure and those who will not.

TABLE 5A
Ability of serum exosomal miRNAs to predict
survival in specific Gleason Grade Groups.

| Serum | | | | | | |
|---|---|---|---|---|---|---|
| exosomal | All | Grade | Grade | Grade | Grade | Grade |
| miRNA | groups | Group 1 | Group 2 | Group 3 | Group 1 + 2 | Group 2 + 3 |
| miR-29a | 0.002* | 0.015* | 0.022 | 0.040* | <0.001* | <0.001* |
| miR-664a | <0.001* | 0.025* | 0.402 | 0.013* | 0.002* | 0.030* |
| miR-331 | 0.838 | 0.576 | 0.009* | 0.621 | 0.451 | 0.034* |
| miR-151-3p | <0.001* | 0.045* | 0.393 | 0.016* | 0.031* | 0.433 |

*statistically significant value

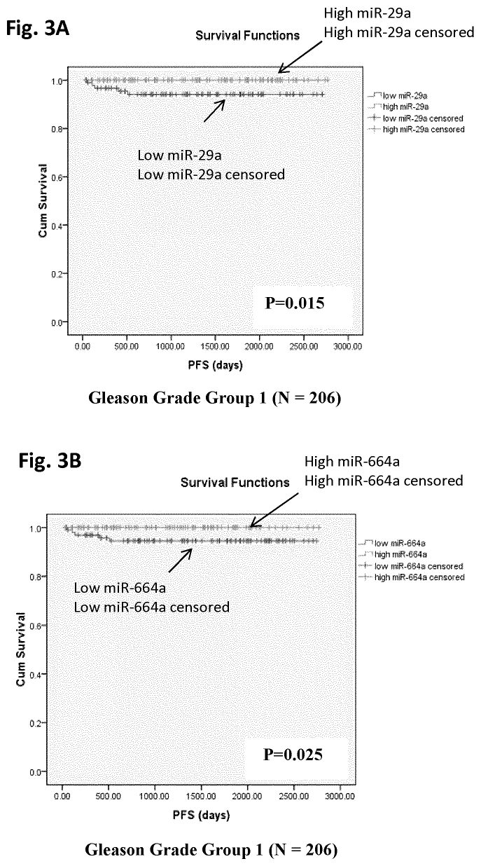

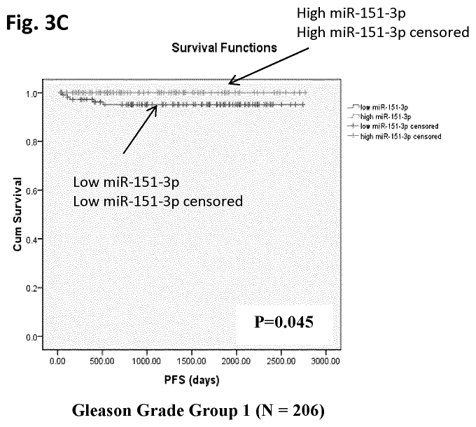

For instance, Gleason Grade Group 1 (Gleason score 3+3=6/10) is always labeled clinically as indolent cancer. However, the present data show that three exosomal miRNAs (miR-29a, miR-664a, and miR-151-3p) were able to distinguish this category into two subgroups (those who will develop relapse vs. no relapse) (p=0.015, 0.025, 0.045, respectively) (FIG. 3). Some members of this group will develop biochemical failure and need to be treated by prostatectomy rather than active surveillance.

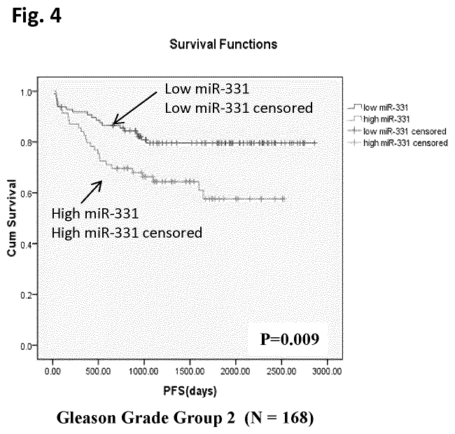

The second challenge is Gleason Grade Group 2 (Gleason score 3+4=7/10). There is no consistent management plan for patients of this group. While some believe that they are still candidates for active surveillance, others suggest that they are candidates for surgical resection. The data showed that miR-331 was able to stratify this into two distinct prognostic subgroups (p=0.009) (FIG. 4).

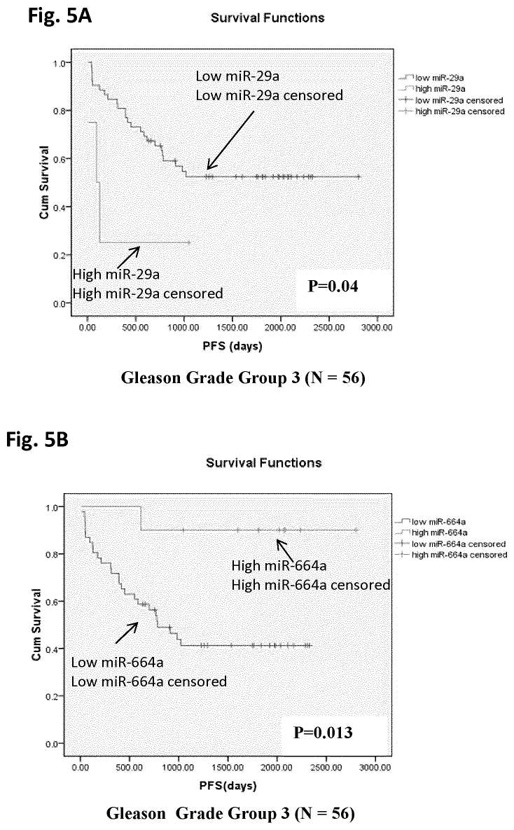

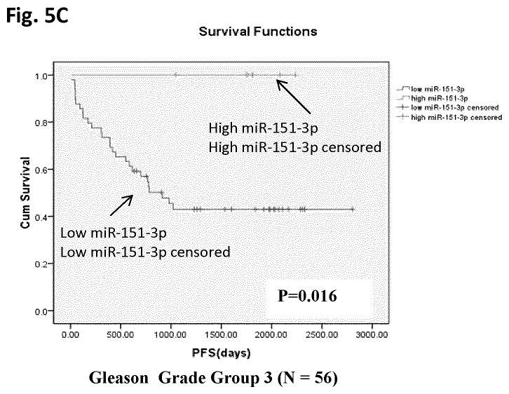

Interestingly, in Gleason Grade Group 3 (Gleason score 4+3=7/10), a subset of this group having indolent disease (non-progressive course) was identified. These patients are candidates for active surveillance rather than surgery. Three exosomal miRNAs (miR-29a, miR-664a, and miR-151-3p) were able to distinguish between the aggressive and non-aggressive patients in this cohort (p=0.040, 0.013, 0.016, respectively). These data suggest that not all patients in this group need radical prostatectomy (FIG. 5).

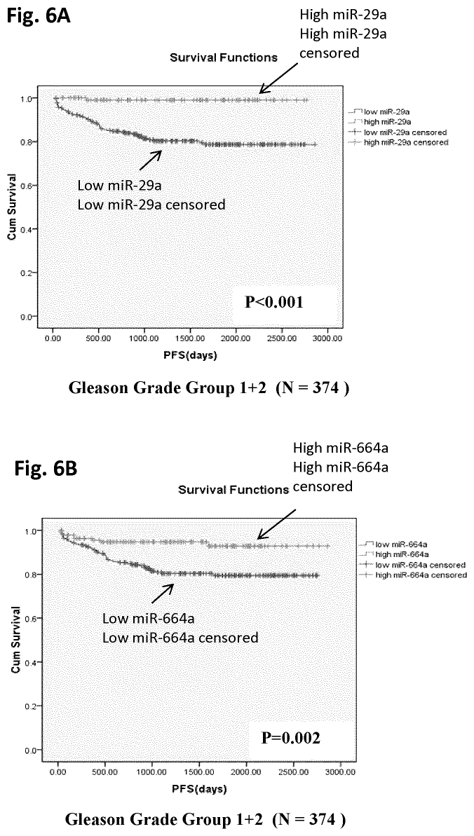

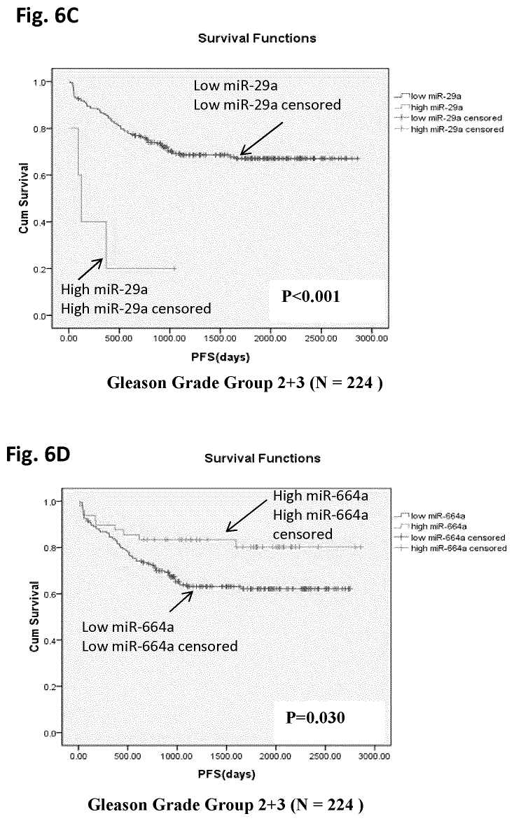

These findings were also valid for a combination of these groups. For example, miR-29a and miR-664a were also able to stratify patients in the combined group 1+2 (p<0.001 and p=0.002, respectively) and the combined group 2+3 (p<0.001 and p=0.030, respectively) into two distinct prognostic subgroups (FIG. 6).

Preoperative Serum Exosomal miRNAs can Predict Gleason Score and Tumor Volume of Prostatectomy

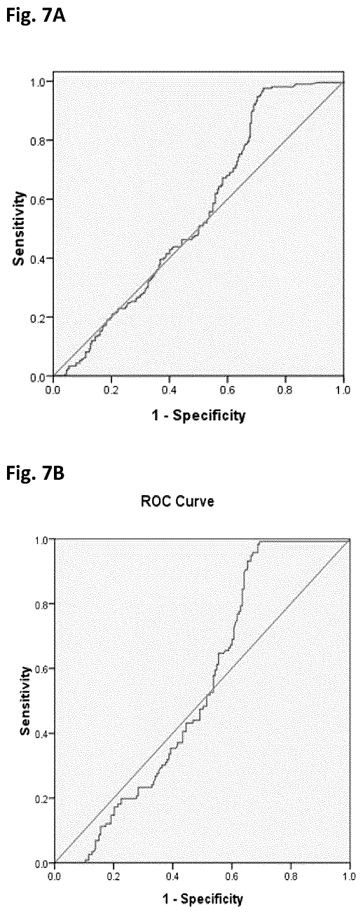

Currently, the decision to actively surveil or perform prostatectomy depends on a number of parameters that are determined from a biopsy. These include tumor volume in the biopsy, and Gleason score of biopsy specimen. However, these parameters do not always provide an accurate assessment of the tumor due to the fact the biopsy may not be generally representative of the entire tumor. The utility of the identified serum exosomal miRNAs to predict disease aggressiveness as indicated by Gleason Grade Group and tumor volume (assessed from the operative specimen which is far more accurate than biopsy) was then determined. In the patient cohort, preoperative serum PSA was not able to distinguish indolent tumors (Gleason≤6) from aggressive ones (Gleason score>6) (p=0.06) (FIG. 7A). The statistics of FIG. 7A are shown in Table 5B. In addition, it was not able to stratify patients according to tumor volume (using 5% as cut-off) (p=0.26) (FIG. 7B). The statistics of FIG. 7B are shown in Table 5C.

TABLE 5B
Statistics from FIG. 7A.

| | | | | 95% CI of AUC | | | |
|---|---|---|---|---|---|---|---|
| | AUC | SD | P | Low | Up | Sensitivity | Specificity |
| PSA | 0.55 | 0.03 | 0.06 | 0.50 | 0.60 | 50.50% | 50.00% |

TABLE 5C
Statistics from FIG. 7B.

| | | | | 95% CI of AUC | | | |
|---|---|---|---|---|---|---|---|
| | AUC | SD | P | Low | Up | Sensitivity | Specificity |
| PSA | 0.54 | 0.03 | 0.26 | 0.47 | 0.61 | 47.40% | 50.90% |

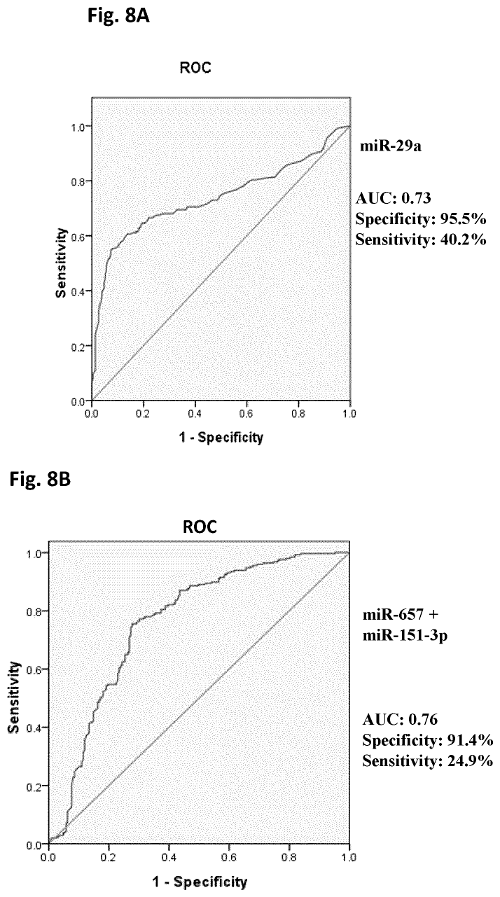

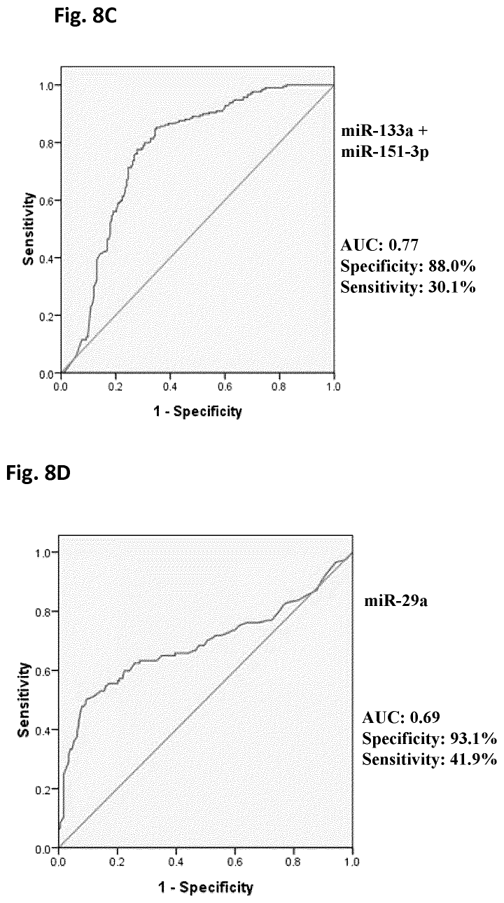

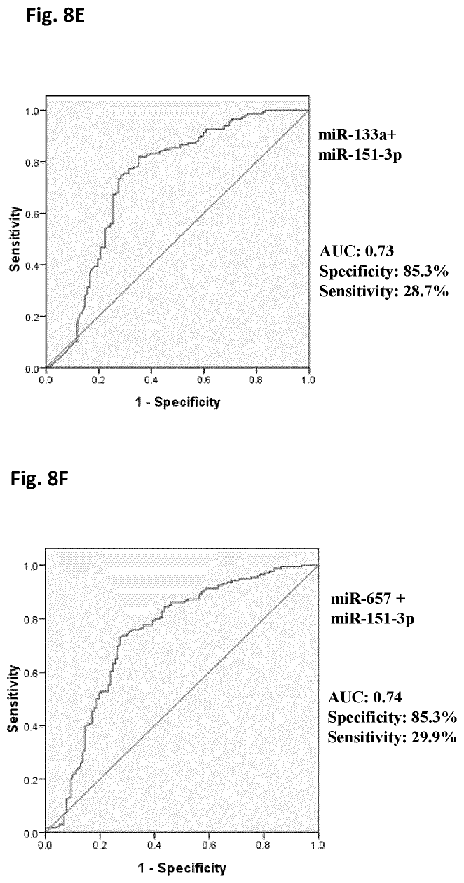

A number of miRNAs having serum exosomal levels able to distinguish Gleason Grade Group 1 from higher grade groups (FIG. 8A) were identified. For example, miR-29a was able to distinguish Gleason Grade Group 1 from higher grade groups (AUC=0.73, specificity=96% and sensitivity is =40%). Moreover, a combination of exosomal miRNAs improved the accuracy of prediction. For instance, the combination of miR-657 and miR-151-3p was able to distinguish between the two populations (AUC=0.76, specificity is =91% and sensitivity is about 25%) (FIG. 8B) as well as the combination of miR-133a and miR-151-3p (AUC=0.77, specificity=88% and sensitivity=30%) (FIG. 8C).

The utility of exosomal miRNA expression levels to predict the combination of Gleason grade and tumor volume in the resection specimen was evaluated. miR-29a was able to distinguish the more indolent tumors (combined Gleason Grade Group 1 and volume <5%) from the more significant ones (combined Gleason Grade Group >1 and volume 5%) (AUC=0.69, specificity is about 93% and sensitivity is about 42%) (FIG. 8D). The model combining miR-133a and miR-151-3p was additionally able to stratify indolent tumors from aggressive ones (AUC=0.73, specificity=85% and sensitivity=29%) (FIG. 8E). Comparable results were obtained from miR-657 and miR-151-3p (AUC=0.74, specificity=85% and sensitivity=30%) (FIG. 8F).

The Survival Prediction Value of Urinary Exosomal miRNAs (Normalized by miR-29a)

The utility of preoperative urinary exosomal miRNAs to predict patients bearing tumors with aggressive behaviors (candidates for prostatectomy) and distinguish them from more indolent cancers (who can be directed to active surveillance) was evaluated. Two approaches were used to normalize the urinary exosomal miRNAs. First, the urinary exosomal miRNAs were normalized by urinary miR-29a. According to the Normfinder software results, miR-29a is the best candidate normalization exosomal miRNA among urine exosomal miRNAs.

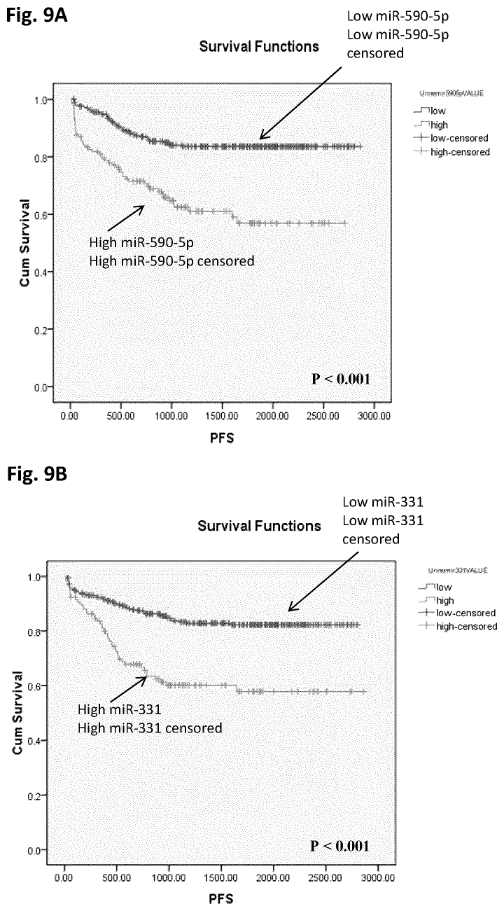

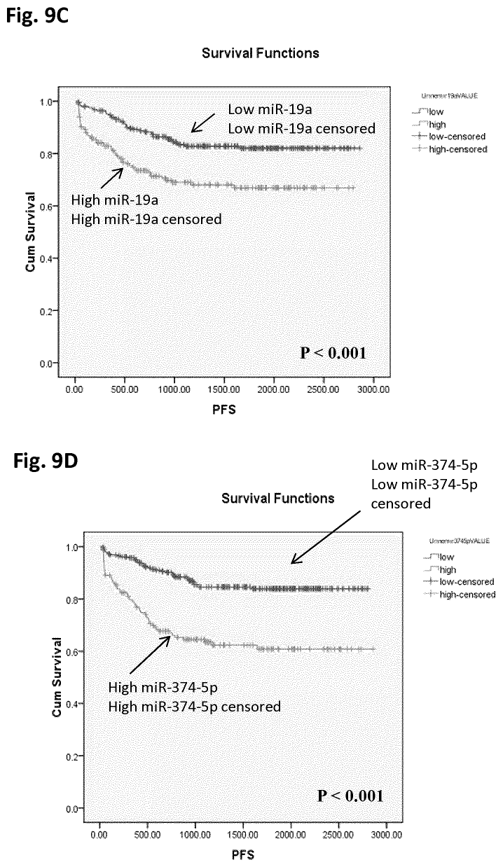

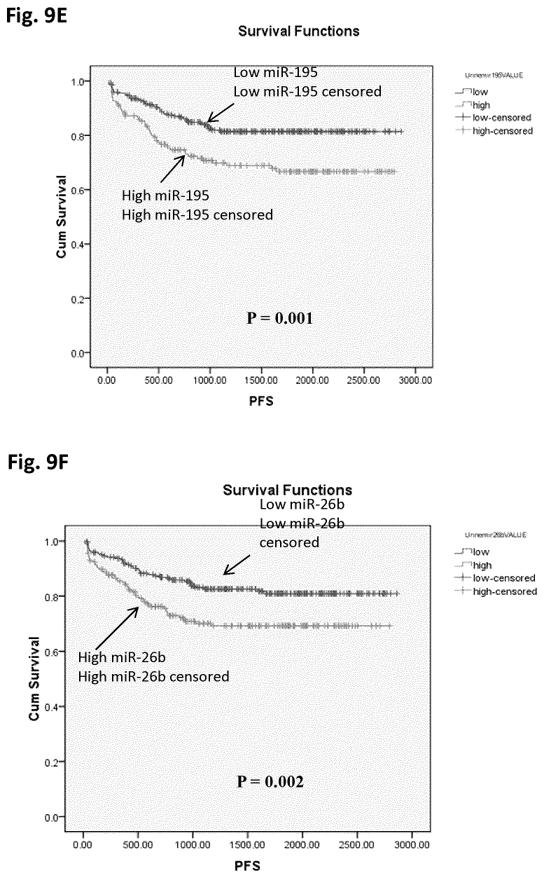

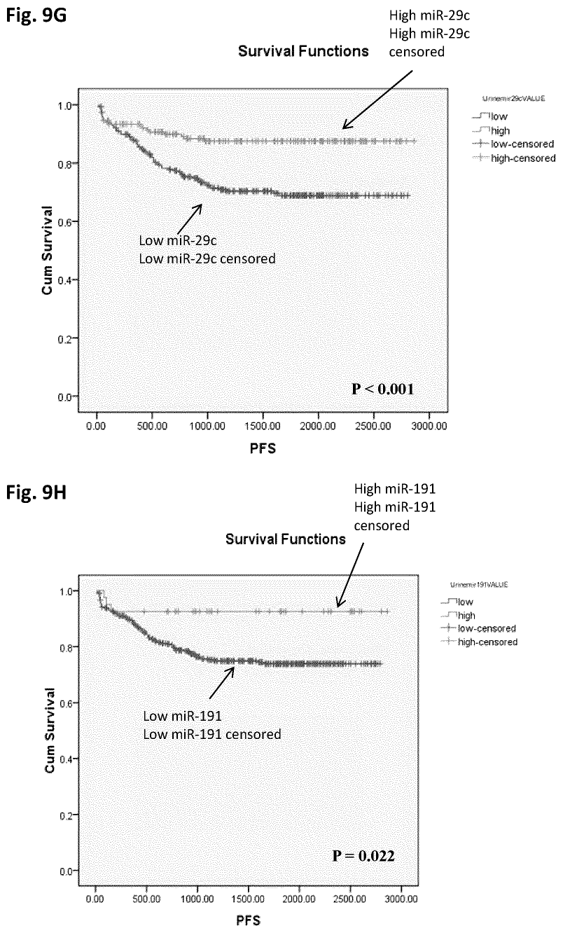

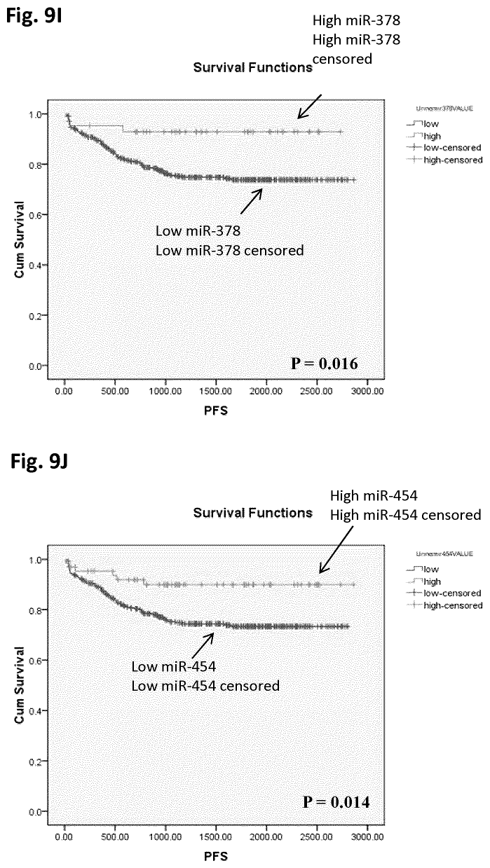

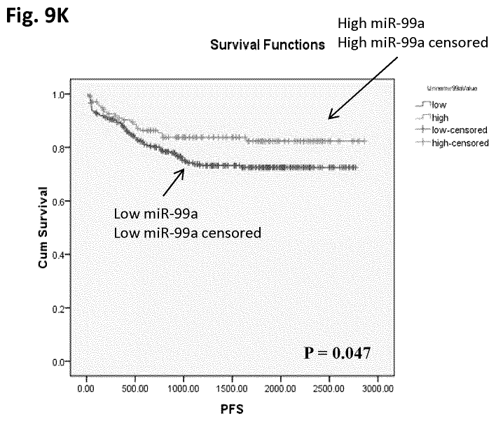

Kaplan-Meier survival analysis showed that eleven urinary exosomal miRNAs were able to predict biochemical failure (normalized by miR-29a) (miR-590-5p, miR-331, miR-19a, miR-374-5p, miR-195, miR-191, miR-26b, miR-29c, miR-378, miR-454 and miR-99a) (p<0.001, p<0.001, p<0.001, p<0.001, p=0.001, p=0.022, p=0.002, p<0.001, p=0.016, p=0.014 and p=0.047) (FIG. 9). Cox regression analysis revealed that miR-19a, miR-29c, miR-590-5p and miR-99a were independent biomarkers to predict biochemical failure after adjusting for Gleason score and tumor volume (p<0.001, p=0.03, p<0.001 and p=0.02) (Table 6).

TABLE 6
Cox Regression analysis of urinary exosomal
miRNAs normalized by miR-29a.

95.0% CI for Exp (B)

| Urine Variables | P | HR | Lower | Upper |
|---|---|---|---|---|
| Gleason | <0.001* | 15.08 | 5.94 | 38.26 |
| Tumor Volume | 0.004* | 2.21 | 1.29 | 3.81 |
| miR-19a | 0.001* | 2.57 | 1.50 | 4.39 |
| miR-29c | 0.03* | 0.55 | 0.32 | 0.95 |
| miR-590-5p | 0.001* | 2.12 | 1.36 | 3.31 |
| miR-99a | 0.02* | 0.53 | 0.31 | 0.92 |
| miR-191 | 0.11 | 0.38 | 0.11 | 1.26 |
| miR-195 | 0.82 | 0.94 | 0.58 | 1.55 |
| miR-26b | 0.43 | 1.23 | 0.74 | 2.06 |
| miR-331 | 0.06 | 1.64 | 0.97 | 2.76 |
| miR-374-5p | 0.05 | 1.66 | 0.99 | 2.79 |
| miR-378 | 0.06 | 0.32 | 0.10 | 1.06 |
| miR-454 | 0.05 | 0.39 | 0.16 | 1.00 |

Urinary Exosomal miRNAs can Predict Disease Relapse in Specific Gleason Grade Groups (Normalized by miR-29a)

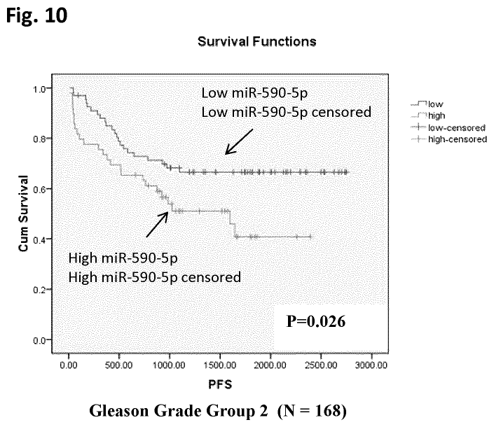

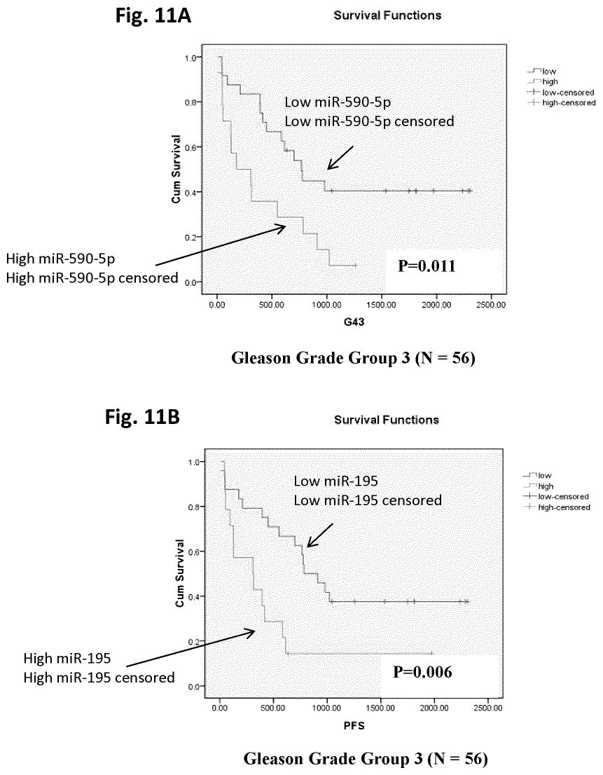

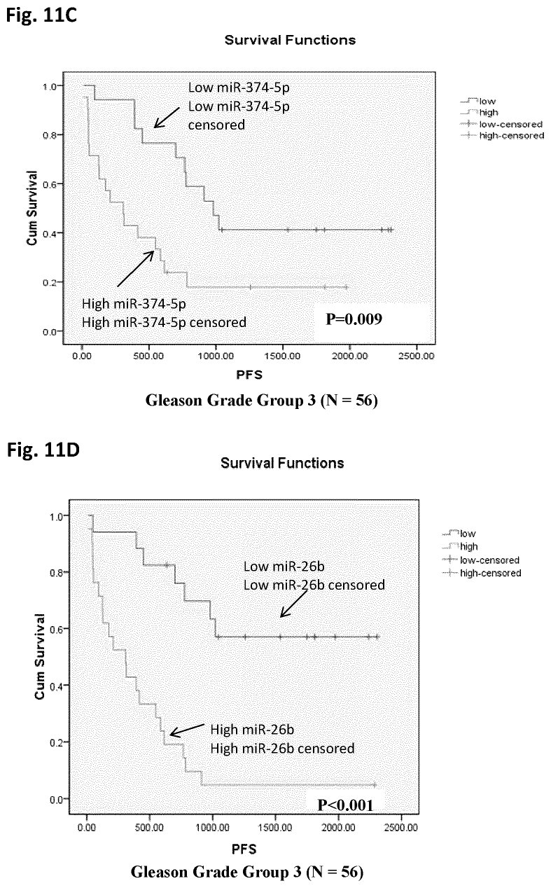

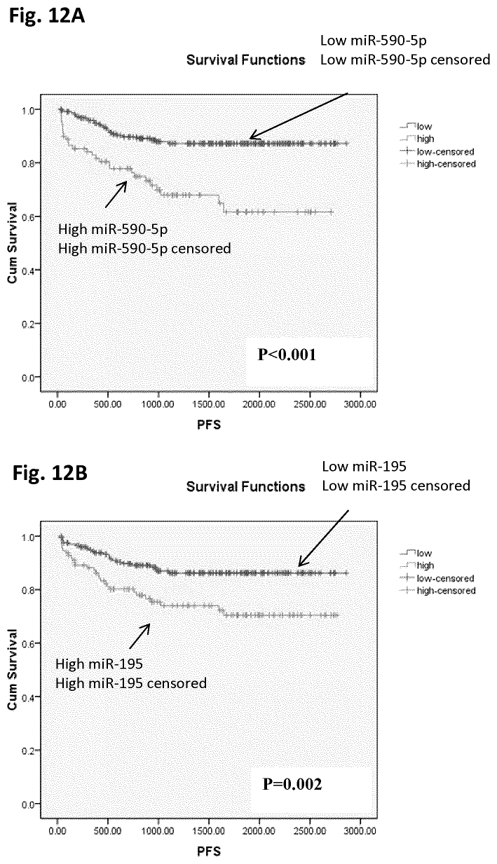

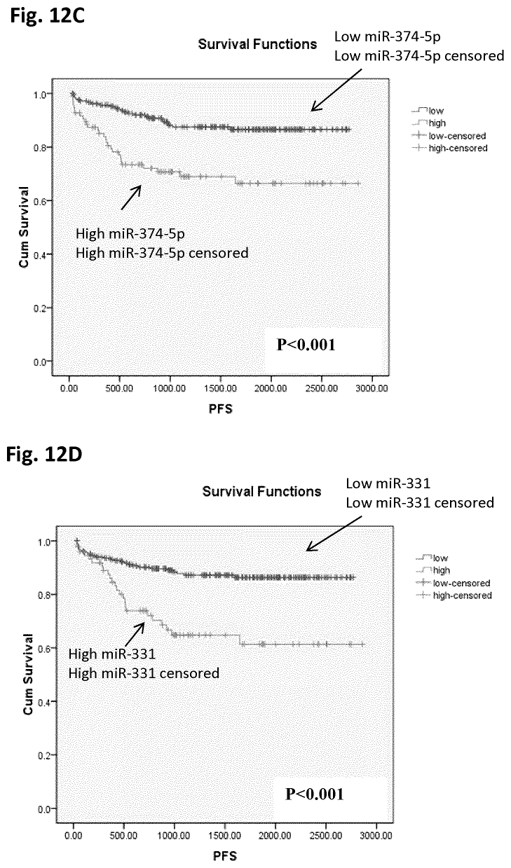

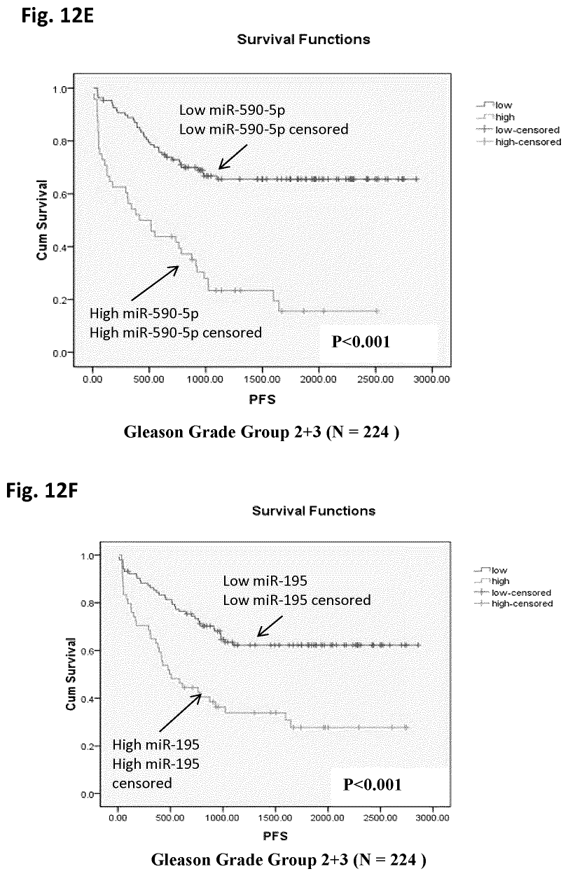

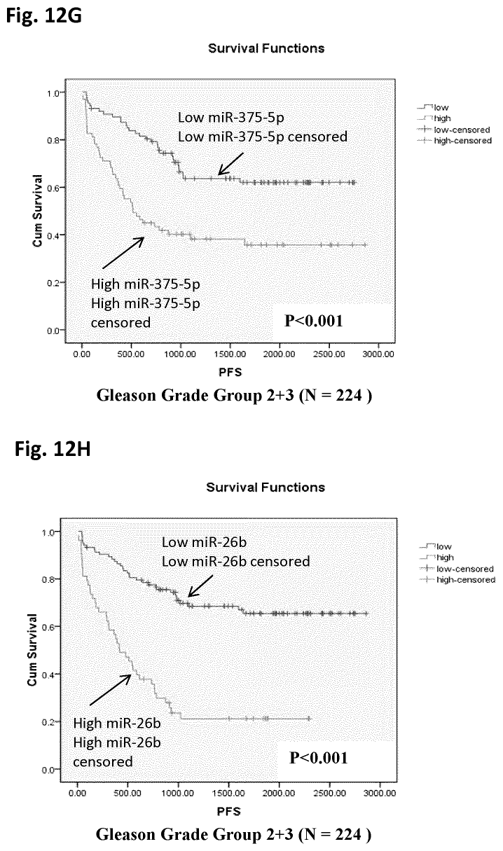

Similar to serum exosomal miRNAs, a number of miRNAs with urinary preoperative exosomal levels predictive of survival outcomes (the possibility of developing biochemical failure after prostatectomy) among specified grade groups were identified. These are miR-590-5p, miR-195, miR-374-5p, miR-26b and miR-331 (Table 7). Data showed that miR-590-5p was able to distinguish Gleason Grade Group 2 (3+4=7/10) into two subgroups (patients who will develop relapse vs. no relapse) (p=0.026) (FIG. 10). In terms of grade group 3, four exosomal miRNAs (miR-590-5p, miR-195, miR-374-5p and miR-26b) could stratify grade group 3 (4+3=7/10) into indolent group and aggressive group (p=0.011, 0.006, 0.009 and p<0.001, respectively) (FIG. 11). These findings were also valid for a combination of these groups. For example, miR-590-5p, miR-195, miR-374-5p and miR-331 were able to stratify patients in the combined group 1+2 (p<0.001, p=0.002, p<0.001 and p<0.001, respectively) into two distinct prognostic subgroups (FIG. 12A-D). Five exosomal miRNAs (miR-590-5p, miR-195, miR-374-5p, miR-26b and miR-331) were shown to be predictive of indolent patients from the combined group 2+3 (p<0.001, p<0.001, p<0.001, p<0.001 and p=0.001, respectively) (FIG. 12E-I).

TABLE 7
Ability of urinary exosomal miRNAs to predict survival in
specific Gleason Grade Groups (normalized by miR-29a).

| | Grade | | | | Grade | Grade |
|---|---|---|---|---|---|---|
| Urine | groups | Grade | Grade | Grade | groups | groups |
| miRNAs | 1-5 | group 1 | group 2 | group 3 | 1 + 2 | 2 + 3 |
| miR-590-5p | <0.001* | 0.556 | 0.026* | 0.011* | <0.001* | <0.001* |
| miR-195 | 0.001* | 0.294 | 0.076 | 0.006* | 0.002* | <0.001* |
| miR-374-5p | <0.001* | 0.406 | 0.061 | 0.009* | <0.001* | <0.001* |
| miR-26b | 0.002* | 0.744 | 0.307 | <0.001* | 0.475 | <0.001* |
| miR-331 | <0.001* | 0.813 | 0.092 | 0.304 | <0.001* | 0.001* |

*statistically significant value

The Survival Predictive Value of Urinary Exosomal miRNAs (Normalized by Creatinine)

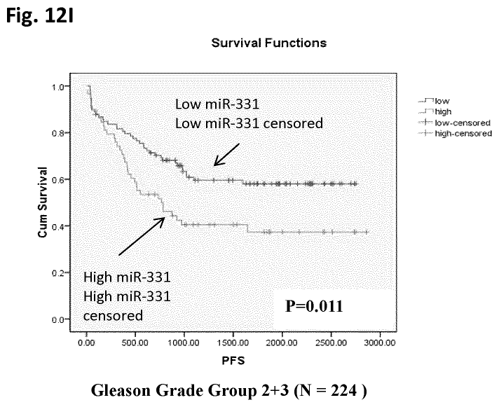

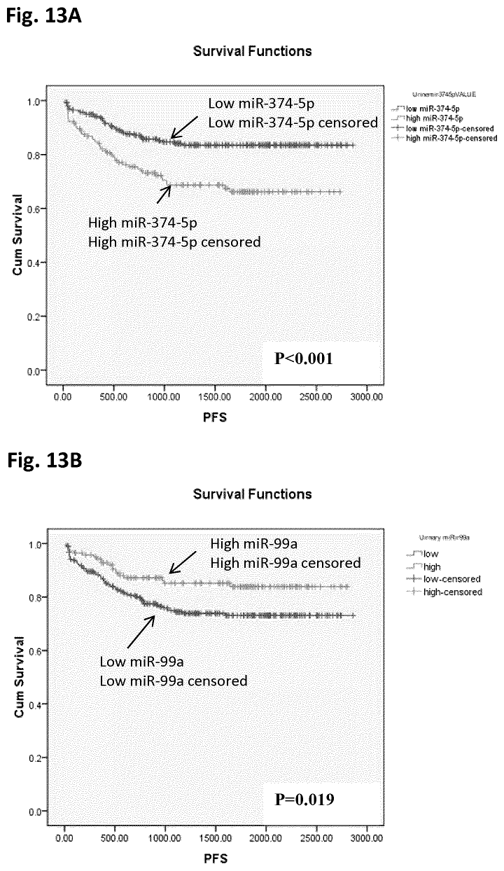

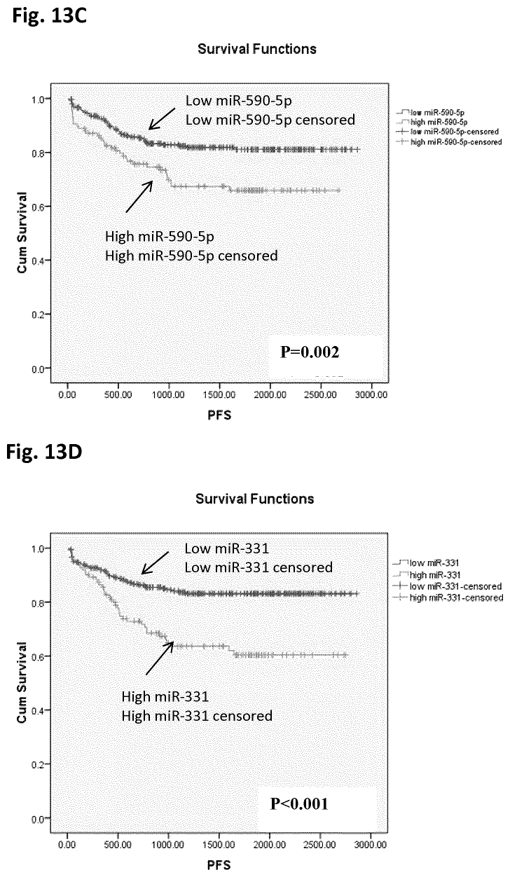

Creatinine was measured as a normalizer for urine exosomal miRNAs. The mean value of urinary creatinine was 4.62 mmol/L (SD=2.83). Using creatinine as a normalizer, Kaplan-Meier survival analysis showed that four urinary exosomal miRNAs were able to predict biochemical failure (miR-374-5p, miR-590-5p, miR-99a and miR-331) (FIG. 13) (p<0.001, p=0.002, p=0.019, and p<0.001, respectively). Cox regression analysis revealed that miR-374-5p and miR-99a were independent biomarkers to predict biochemical failure after adjusting for Gleason score and tumor volume (p<0.001 and p=0.03, respectively) (Table 8).

TABLE 8
Cox regression analysis of urinary exosomal
miRNAs (normalized by creatinine).

| | |
|---|---|
| Urine | 95.0% CI |

| Variables | P | HR | Low | High |
|---|---|---|---|---|
| Gleason | 0.009* | 2.09 | 1.21 | 3.64 |
| score | | | | |
| Tumor | <0.001* | 10.25 | 4.10 | 25.63 |
| volume | | | | |
| miR-374-5p | 0.03* | 2.11 | 1.08 | 4.13 |
| miR-99a | <0.001* | 0.34 | 0.19 | 0.60 |
| miR-590-5p | 0.195 | 1.47 | 0.82 | 2.62 |
| miR-331 | 0.413 | 1.28 | 0.71 | 2.32 |

*statistically significant value

Urinary Exosomal miRNAs Predict Survival in Gleason Grade Groups (Normalized by Creatinine)

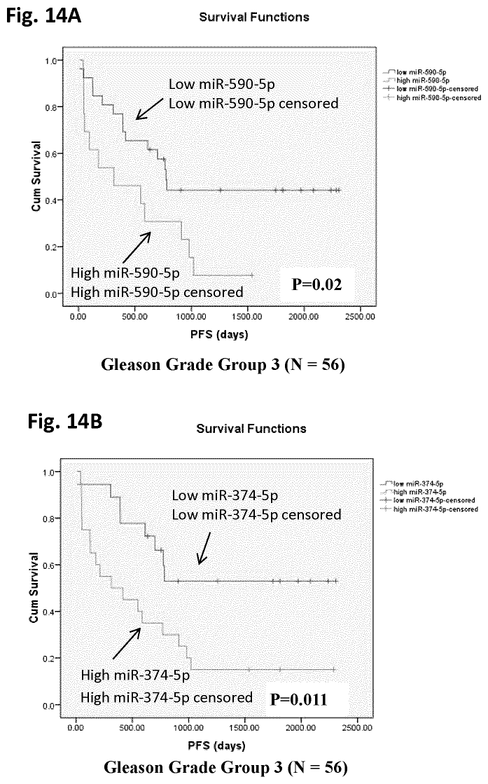

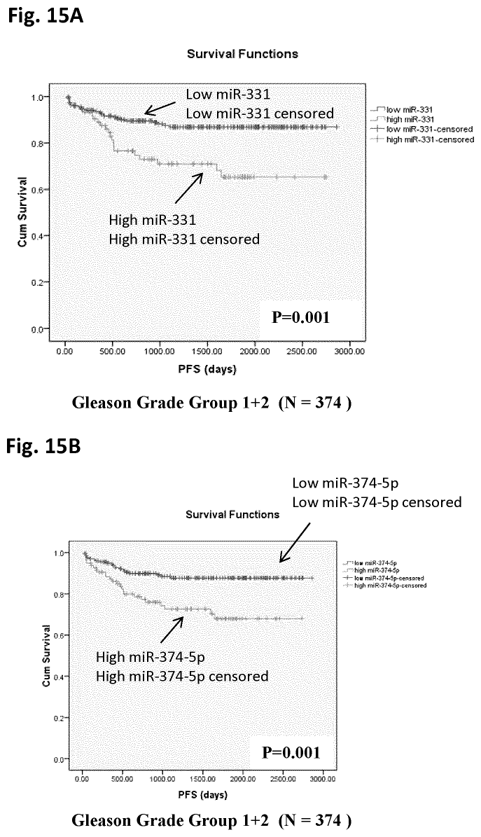

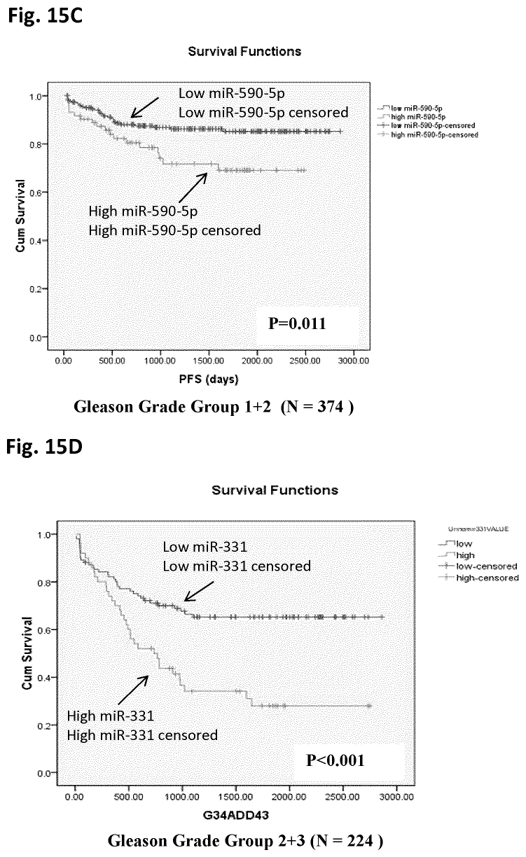

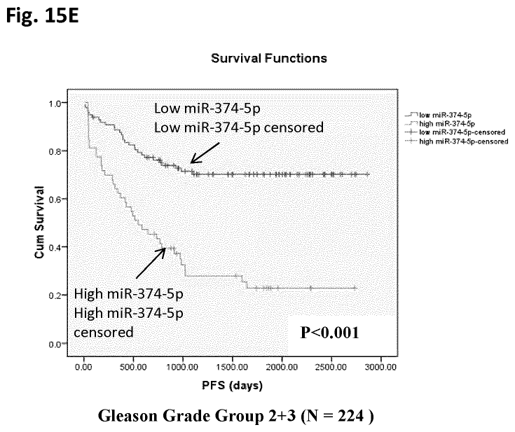

The data showed that urinary exosomal miRNAs are predictive of survival in specific Gleason Grade Groups (Table 9). A number of exosomal miRNAs were found to be useful as predictive biomarkers in specific groups. For Gleason Grade Group 3 (4+3=7/10), miR-374-5p and miR-590-5p were able to distinguish the indolent tumors from aggressive ones (p=0.011 and 0.020, respectively) (FIG. 14). miR-331, miR-374-5p and miR-590-5p can stratify indolent patients from combined Gleason Grade Groups 1+2 (p=0.001, 0.001 and 0.011, respectively). For the combined group of patients with Gleason Grade Groups 2+3, exosomal miRNAs miR-331 and miR-374-5p were also useful in distinguishing indolent patients from those suffering from aggressive disease (both p<0.001) (FIG. 15).

TABLE 9
Ability of urinary exosomal miRNAs to predict survival in
specific Gleason Grade Groups (normalized by creatinine).

| Urine | Grade | | | | Grade | Grade |
|---|---|---|---|---|---|---|
| exosomal | groups | Grade | Grade | Grade | groups | groups |
| miRNAs | 1-5 | group 1 | group 2 | group 3 | 1 + 2 | 2 + 3 |
| miR-331 | <0.001* | 0.330 | 0.139 | 0.07 | 0.001* | <0.001* |
| miR-374-5p | <0.001* | 0.115 | 0.35 | 0.011* | 0.001* | <0.001* |
| miR-590-5p | 0.001* | 0.781 | 0.647 | 0.020* | 0.011* | <0.001* |

*statistically significant value

Taken together, preoperative urinary exosomal miRNAs could stratify each of the Gleason Grade Groups in a more objective way. For each group, there is a subset of patients who have more aggressive disease despite having the same Gleason grade, and these patients may be referred to prostatectomy, whereas the rest of the patients have indolent disease and are potential candidates for active surveillance.

Development of Models Combining PSA, Exosomal Serum miRNAs and Urinary miRNAs for Assessment of Both Gleason Score and Tumor Volume in Prostatectomy

Accurate prediction of the Gleason Grade Group and tumor volume that are measured in prostatectomy specimen (rather than biopsy) is involved in making the objective decision of prostatectomy vs. active surveillance.

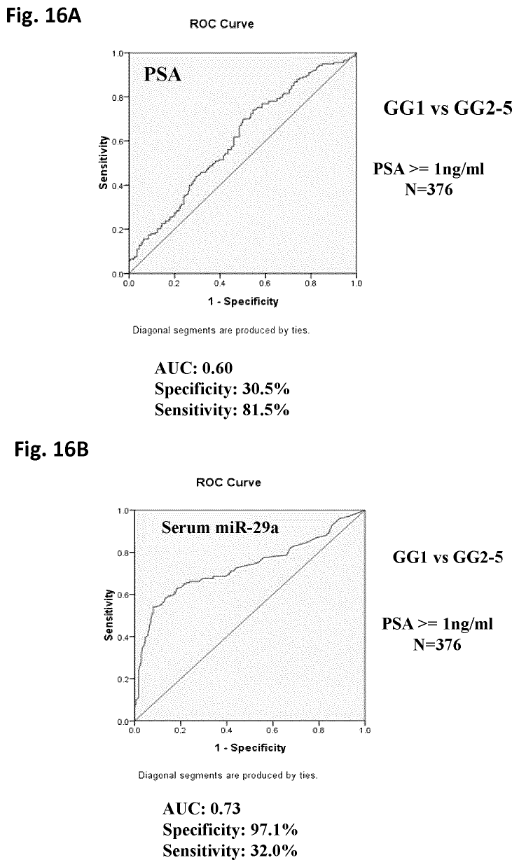

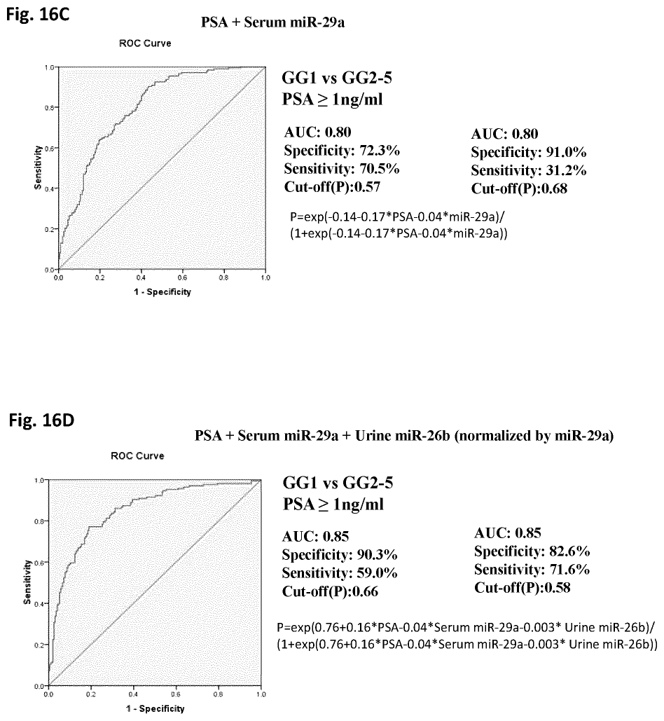

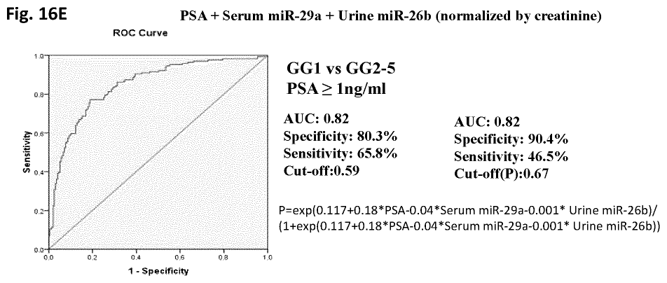

Further assessment of the patient cohort was performed focusing on the subgroup of patients with preoperative serum PSA value 1.0 ng/ml (excluding the patients with PSA <1 ng/ml with diagnosed cancer). In this subgroup of patients, preoperative serum PSA was not able to distinguish grade group 1 from higher ones (AUC=0.60, specificity=31% and sensitivity=82%, FIG. 16A). Meanwhile, the data showed that serum miR-29a was able to distinguish these two groups (AUC=0.73, specificity=97% and sensitivity=32%, FIG. 16B). In order to improve the model, serum PSA was combined with a number of exosomal miRNAs, as shown in Table 10. Combining serum PSA with serum miR-29a resulted in improved test performance (AUC=0.80, specificity=72% and sensitivity=71%, FIG. 16C). Probability (P) is generated according to the formula: P=exp(−0.14−0.17*PSA-0.04*miR-29a)/(1+exp(−0.14−0.17*PSA-0.04*miR-29a)). The probability (P) value was compared with the cut-off (P) value in ROC analysis. This model provides balanced specificity and sensitivity. Some clinicians prefer higher specificity, even at the expense of slightly increased false negative results since these patients will not be lost for follow-up by active surveillance. Adjusting the cut-off value can lead to an enhancing specificity (91%) with a lower sensitivity (31%) (FIG. 16C). The skilled person in the art can readily adjust the cut-off (P) to vary the specificity and sensitivity requirements.

TABLE 10
Combined models for the distinguishing of Gleason Grade Group 1
from higher grade groups (serum PSA value more than 1.0 ng/mL).

| Models | AUC | Specificity | Sensitivity | Cut-off |
|---|---|---|---|---|
| Serum miR-29a | 0.73 | 97% | 32% | 35.04 |
| | | | | Copies/ul |
| | | | | serum |
| PSA + Serum miR-29a | 0.80 | 91% | 31% | 0.68 |
| PSA + Serum miR-29a | 0.80 | 72% | 71% | 0.57 |
| PSA + Serum miR-29a + Urine | 0.85 | 90% | 59% | 0.66 |
| miR-26b (normalized | | | | |
| by miR-29a) | | | | |
| PSA + Serum miR-29a + Urine | 0.85 | 83% | 72% | 0.58 |
| miR-26b (normalized | | | | |
| by miR-29a) | | | | |
| PSA + Serum miR-29a + Urine | 0.82 | 90% | 47% | 0.67 |
| miR-26b (normalized | | | | |
| by creatinine) | | | | |
| PSA + Serum miR-29a + Urine | 0.82 | 80% | 66% | 0.59 |
| miR-26b (normalized | | | | |
| by creatinine) | | | | |

Adding urinary miR-26b (normalized by miR-29a) to the combined model described above generated better AUC (0.85), specificity (83%) and sensitivity (72%) than the two-exosomal miRNA model (normalized by miR-29a) (Table 10, FIG. 16D). Probability (P) is generated according to the formula: P=exp(0.76+0.16*PSA-0.04*Serum miR-29a-0.003* Urine miR-26b)/(1+exp(0.76+0.16*PSA-0.04*Serum miR-29a-0.003* Urine miR-26b)). When normalized using creatinine, the three-exosomal miRNA model generated AUC of 0.82, specificity of 80% and sensitivity of 66% (Table 10, FIG. 16E). Probability (P) is generated according to the formula: P=exp(0.117+0.18*PSA-0.04*Serum miR-29a-0.001* Urine miR-26b)/(1+exp(0.117+0.18*PSA-0.04*Serum miR-29a-0.001* Urine miR-26b)).

For distinguishing tumors with both grade group 1 and tumor volume <5%, it was determined that a model combining PSA, serum miR-29a and urinary miR-26b was highly efficient (Table 11, FIG. 17). When normalized using miR-29a, the three-exosomal miRNA model generated AUC of 0.83, specificity of 78% and sensitivity of 71% (FIG. 17A). Probability (P) is generated according to the formula: P=exp(0.52+0.22*PSA-0.03*Serum miR-29a-0.003* Urine miR-26b)/(1+exp(0.52+0.22*PSA-0.03*Serum miR-29a-0.003* Urine miR-26b)). When levels of urine exosomal miRNAs were normalized to creatinine, the model generated AUC of 0.82, specificity of 81% and sensitivity of 71% (FIG. 17B). Probability (P) is generated according to the formula: P=exp(−0.13+0.25*PSA-0.03*Serum miR-29a-0.002* Urine miR-26b)/(1+exp(−0.13+0.25*PSA-0.03*Serum miR-29a-0.002* Urine miR-26b)).

TABLE 11
Combined models for the distinguishing of indolent from
aggressive groups (serum PSA value > 1.0 ng/mL).

| Models | AUC | Specificity | Sensitivity | Cut-off |
|---|---|---|---|---|
| PSA + Serum miR-29a + Urine | 0.83 | 91% | 60% | 0.68 |
| miR-26b (normalized | | | | |
| by miR-29a) | | | | |
| PSA + Serum miR-29a + Urine | 0.83 | 78% | 71% | 0.62 |
| miR-26b (normalized | | | | |
| by miR-29a) | | | | |
| PSA + Serum miR-29a + Urine | 0.82 | 90% | 51% | 0.68 |
| miR-26b (normalized | | | | |
| by creatinine) | | | | |
| PSA + Serum miR-29a + Urine | 0.82 | 81% | 71% | 0.58 |
| miR-26b (normalized | | | | |
| by creatinine) | | | | |

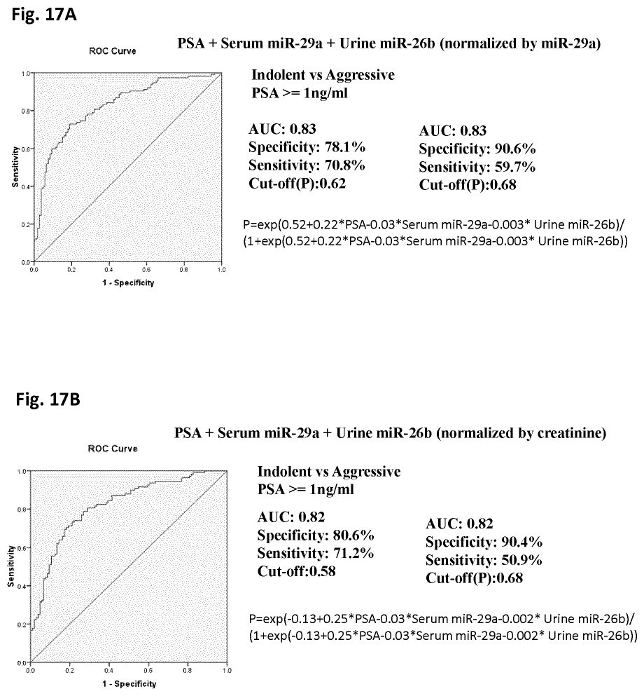

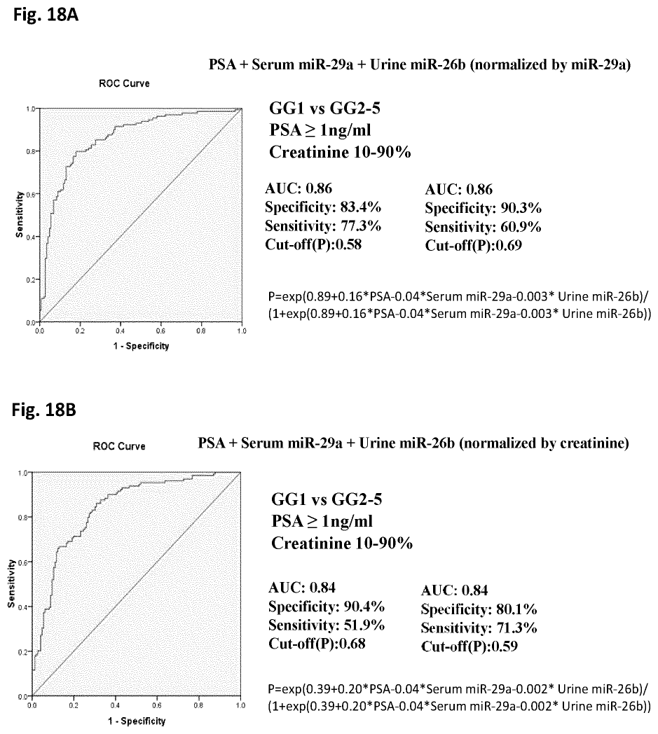

Analysis of the subgroup of patients with creatinine level between 10-90% (1.31-8.42 mmol/L) as well as PSA1 ng/ml showed that the combined model described above can distinguish low from high Gleason Grade Group tumors with both high specificity (83% or 80%) and high sensitivity (77% or 71%) (urinary exosomal miRNAs normalized by miR-29a or creatinine, respectively) (Table 12, FIG. 18). Probability (P) for which urine exosomal miRNAs are normalized by miR-29a is generated by the formula: P=exp(0.89+0.16*PSA-0.04*Serum miR-29a-0.003* Urine miR-26b)/(1+exp(0.89+0.16*PSA-0.04*Serum miR-29a-0.003* Urine miR-26b)). Probability (P) for which urine exosomal miRNAs are normalized by creatinine is generated by the formula: P=exp(0.39+0.20*PSA-0.04*Serum miR-29a-0.002* Urine miR-26b)/(1+exp(0.39+0.20*PSA-0.04*Serum miR-29a-0.002* Urine miR-26b)).

TABLE 12
Combined models for the distinguishing of Gleason
Grade Group 1 from higher grade groups (serum PSA
value > 1.0 ng/mL and creatinine range 10-90%).

| Models | AUC | Specificity | Sensitivity | Cut-off |
|---|---|---|---|---|
| PSA + Serum miR-29a + Urine | 0.86 | 90% | 61% | 0.69 |
| miR-26b (normalized | | | | |
| by miR-29a) | | | | |
| PSA + Serum miR-29a + Urine | 0.86 | 83% | 77% | 0.58 |
| miR-26b (normalized | | | | |
| by miR-29a) | | | | |
| PSA + Serum miR-29a + Urine | 0.84 | 90% | 52% | 0.68 |
| miR-26b (normalized | | | | |
| by creatinine) | | | | |
| PSA + Serum miR-29a + Urine | 0.84 | 80% | 71% | 0.59 |
| miR-26b (normalized | | | | |
| by creatinine) | | | | |

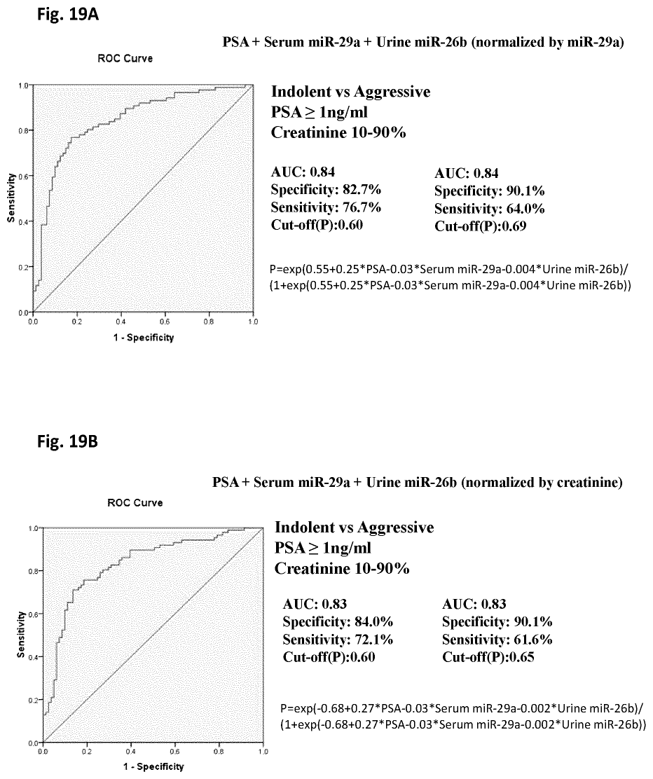

For the prediction of combined low group grade and low volume, the model also could reach specificity of 83% or 84% and sensitivity of 77% or 72% (using miR-29a or creatinine as normalizer for urinary exosomal miRNAs) (Table 13, FIG. 19). Probability (P) for which urine exosomal miRNAs are normalized by miR-29a is generated by the formula: P=exp(0.55+0.25*PSA-0.03*Serum miR-29a-0.004* Urine miR-26b)/(1+exp(0.55+0.25*PSA-0.03*Serum miR-29a-0.004* Urine miR-26b)). Probability (P) for which urine exosomal miRNAs are normalized by creatinine is generated by the formula: P=exp(−0.68+0.27*PSA-0.03*Serum miR-29a-0.002* Urine miR-26b)/(1+exp(−0.68+0.27*PSA-0.03*Serum miR-29a-0.002* Urine miR-26b)).

TABLE 13
Combined models for distinguishing indolent tumors from
aggressive tumors (serum PSA value > 1.0 ng/mL and
creatinine range 10-90%).

| Models | AUC | Specificity | Sensitivity | Cut-off |
|---|---|---|---|---|
| PSA + Serum miR-29a + Urine | 0.84 | 90% | 64% | 0.69 |
| miR-26b (normalized | | | | |
| by miR-29a) | | | | |
| PSA + Serum miR-29a + Urine | 0.84 | 83% | 77% | 0.60 |
| miR-26b (normalized | | | | |
| by miR-29a) | | | | |
| PSA + Serum miR-29a + Urine | 0.83 | 90% | 62% | 0.65 |
| miR-26b (normalized | | | | |
| by creatinine) | | | | |
| PSA + Serum miR-29a + Urine | 0.83 | 84% | 72% | 0.60 |
| miR-26b (normalized | | | | |
| by creatinine) | | | | |

### Risk Stratification Model A

A multiparametric biomarker improved performance (Tables 14-16). Combining PSA with 1) preoperative urine (Table 14) and 2) serum (Table 15) exosomal miRNAs (urine and serum combined in Table 16), a risk stratification model was developed to provide prediction of two factors critical (i.e. disease relapse and Gleason grade score) for treatment decision making. The model predicts probability of disease relapse (biochemical failure) before prostatectomy (c-index 0.89) (Table 16). The model predicts either disease relapse or high Gleason grade considered as high risk score. For continuous predictor such as age and PSA, the “unit” shown in Tables 14-16 is for one digit of the measurement, which is relating to weighing in generating a c-index.

TABLE 14
Urine miRNAs model. C-index for the model
combining PSA urine miRNA markers is 0.88.

| Covariate | HR (95% CI) | | Global p-value |
|---|---|---|---|
| Pre op Total PSA 100 unit | 0.22 | (0.15, 0.33) | <0.001 |
| miR-331 u 10 unit | 1.09 | (1, 1.19) | 0.054 |
| miR-99a u 10 unit | 0.51 | (0.33, 0.8) | 0.0036 |
| miR-374 5p u 10 unit | 1.2 | (1.11, 1.31) | <0.001 |
| miR-454 u 10 unit | 0.66 | (0.51, 0.85) | 0.0016 |
| miR-195 u 10 unit | 1.11 | (1.06, 1.16) | <0.001 |
| miR-29c u 10 unit | 0.88 | (0.82, 0.93) | <0.001 |
| miR-26b u 10 unit | 1.02 | (1.01, 1.04) | 0.007 |
| miR-590-5p u 10 unit | 1.29 | (1.09, 1.54) | 0.004 |

HR = hazard ratio.

TABLE 15
Serum miRNAs model. C-index for the model
combining PSA serum miRNA markers is 0.87.

| Covariate | HR (95% CI) | | Global p-value |
|---|---|---|---|
| Pre op Total PSA 100 unit | 0.17 | (0.11, 0.25) | <0.001 |
| miR-875 3p s 10 unit | 0.4 | (0.2, 0.79) | 0.0083 |
| miR-34a s 10 unit | 1.21 | (1.03, 1.42) | 0.022 |
| miR-331 s 10 unit | 1.43 | (1.23, 1.66) | <0.001 |
| miR-664a s 10 unit | 0.48 | (0.29, 0.8) | 0.0046 |
| miR-29a s 10 unit | 0.87 | (0.77, 0.97) | 0.016 |

HR = hazard ratio.

TABLE 16
Combined serum and urine miRNAs model with PSA.
C-index for the model combining serum and
urine miRNA markers with PSA is 0.89.

| Covariate | HR (95% CI) | | Global p-value |
|---|---|---|---|
| Pre op Total PSA 100 unit | 0.3 | (0.21, 0.43) | <0.001 |
| Scored Urine | 1.94 | (1.59, 2.36) | <0.001 |
| Scored Serum | 1.99 | (1.5, 2.65) | <0.001 |

HR = hazard ratio.

Risk Stratification Model B

A multiparametric biomarker improved performance (Tables 17-19). Combining PSA and age with 1) preoperative urine (Table 17) and 2) serum (Table 18) exosomal miRNAs (urine and serum combined in Table 19), a risk stratification model was developed to provide prediction of two factors critical (i.e. disease relapse and Gleason grade score) for treatment decision making. The model predicts probability of disease relapse (biochemical failure) before prostatectomy (c-index 0.89) (Table 16). It is also the first to predict the Gleason score of the entire tumor with higher accuracy (C-index 0.81, compared to 0.61 for PSA alone) (Table 19). For continuous predictor such as age and PSA, the “unit” shown in Tables 17-19 is for one digit of the measurement. For example, for age, one unit means one year; 10 units mean 10 years.

TABLE 17
Urine miRNAs model. AUC for the model combining
PSA, age and urine miRNA markers is 0.77.

| Covariate | OR (95% CI) | | Global p-value |
|---|---|---|---|
| Pre op Total PSA 100 unit | 0.86 | (0.65, 1.13) | 0.27 |
| AGE AT radical prostatectomy | 1.06 | (1.01, 1.11) | 0.01 |
| miR-331 u 10 unit | 1.22 | (1.04, 1.42) | 0.013 |
| miR-34a u 10 unit | 1.61 | (1.13, 2.3) | 0.0091 |
| miR-99a u 10 unit | 0.42 | (0.24, 0.74) | 0.0026 |
| miR-374 5p u 10 unit | 1.15 | (1.04, 1.28) | 0.0087 |
| miR-454 u 10 unit | 0.74 | (0.54, 1.02) | 0.063 |
| miR-133b expression Copies | 1.67 | (0.96, 2.89) | 0.069 |
| 20 ul u 10 unit | | | |

OR = odds ratio.

TABLE 18
Serum miRNAs model. AUC for the model combining
PSA, age and serum miRNA markers is 0.75.

| Covariate | OR (95% CI) | | Global p-value |
|---|---|---|---|
| Pre op Total PSA 100 unit | 0.73 | (0.56, 0.95) | 0.018 |
| AGE AT RP | 1.07 | (1.03, 1.12) | 0.0016 |
| miR-875 3p s 10 unit | 0.38 | (0.15, 0.97) | 0.042 |
| miR-34a s 10 unit | 1.39 | (1.13, 1.73) | 0.0023 |
| miR-664a s 10 unit | 0.25 | (0.1, 0.62) | 0.0026 |
| miR-19b s 10 unit | 4.13 | (1.25, 13.65) | 0.02 |

OR = odds ratio.

TABLE 19
Combined urine and serum miRNAs model. AUC
for the model combining PSA, age and combined
urine and serum miRNA markers is 0.81.

| Covariate | OR (95% CI) | Global p-value |
|---|---|---|
| Pre op Total PSA 100 unit | 0.97 (0.73, 1.29) | 0.84 |
| AGE AT RP | 1.06 (1.01, 1.11) | 0.012 |
| Scored Urine | 2.43 (1.72, 3.43) | <0.001 |
| Scored Serum | 2.53 (1.65, 3.87) | <0.001 |

OR = odds ratio.

### Discussion

The present disclosure has a number of advantages. This is the first non-invasive serum and urinary test for preoperative assessment of tumor aggressiveness (including the likelihood to develop biochemical failure, and the prostatectomy Gleason score and tumors volume). As a non-invasive test, presently disclosed biomarkers are much more accurate in reflecting the entire lesion compared to the biopsy specimen which is not always representative of the entire lesion area. Without wishing to be bound by theory, it is proposed that exosomal miRNA are secreted, thus reflecting different tumors grades and aggressive lesions and will overcome the problem of tumor heterogeneity.

In addition, the present inventors rely on exosomal miRNAs which are reliable molecules. There are many advantages in using miRNAs as unique biomarkers. These include their stability (they are resistant to degradation in formalin-fixed tissues and bio-fluids). Exosomal miRNAs are also actively secreted, making the measurement more consistent. Moreover, methods described herein focused on exosomal miRNAs, which are predicted to have less variability among the runs since exosomal secretion is a physiologically controlled process. The present disclosure is the first preoperative non-invasion test to aid treatment decision.

Recent reports have shown that assessing changes in miRNA expression in prostate cancer represent promising potential biomarkers that can aid in the diagnosis and assessment of prognosis of prostate cancer. miRNA expression profile has been screened in prostate cancer paraffin tissues and expression compared between patients with high vs. low Gleason grades and between those with and without biochemical failure^{15, 16}. In this Example, clinical utility of a number of exosomal miRNAs as serum/urine non-invasive biomarkers was examined.

Although PSA has been a useful prostate cancer biomarker for clinical applications, it has significant limitations in reflecting the aggressiveness of prostate lesion; patients have been over diagnosed and over-treated. Preoperative PSA value does not accurately predict disease prognosis, as shown in FIG. 1A. Operative Gleason score is an accurate index to reflect disease aggressiveness, but cannot aid in avoiding invasive prostatectomy. Tissue biopsies are invasive but less accuracy since it only samples part of the tumor.

Currently, there are a number of molecular markers that are commercially available for prognostic prediction in prostate cancer, including the Cell Cycle Progression score from Prolaris, Genomics Predictor Score (GPS)™ from Oncotype DX, Genomic Classifier (GC)™ from Decipher. All these rely on measuring RNA, in particular RNA mostly from formalin-fixed tissues, making these approaches inaccurate. In addition, most are only able to predict prognosis postoperatively on prostatectomy specimen, limiting their value as preoperative tests to guide the treatment decision.

The only kit that is recently available as a non-invasive test in prostate cancer is the PROGENSA PCA3™ Assay, which is used to aid in the decision for repeat biopsy in men 50 years of age or older who have had one or more previous negative prostate biopsies. For example, this kit helps to make the decision whether there is value of re-biopsy if a patient has a high PSA but a negative biopsy that might have been resulted from a hidden cancer that was not detected in the first instance, i.e. false negative.

In the methods described herein, for analysis, the highly specific and sensitive Droplet Digital PCR (ddPCR) may be used. This technology has unprecedented sensitivity in addition to the ability for absolute quantification, resulting in great reproducibility. Moreover, ddPCR overcomes most PCR problems, including the bias introduced during the pre-amplification. The skilled person readily recognizes suitable alternative techniques or instrumentations that provide sufficient specificity and sensitivity in quantifying miRNA levels.

While the present disclosure has been described with reference to what are presently considered to be the preferred example, it is to be understood that the disclosure is not limited to the disclosed example. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1 Al-Qatati A, Akrong C, Stevic I, Pantel K, Awe J, Saranchuk J et al. Plasma microRNA signature is associated with risk stratification in prostate cancer patients. International journal of cancer 2017.
2 Buyyounouski M K, Choyke P L, McKenney J K, Sartor O, Sandler H M, Amin M B et al. Prostate cancer—major changes in the American Joint Committee on Cancer eighth edition cancer staging manual. C A: a cancer journal for clinicians 2017; 67: 245-253.
3 Calin G A, Croce C M. MicroRNA signatures in human cancers. Nature reviews Cancer 2006; 6: 857-866.
4 Cooperberg M R, Cowan J E, Hilton J F, Reese A C, Zaid H B, Porten S P et al. Outcomes of active surveillance for men with intermediate-risk prostate cancer. Journal of clinical oncology: official journal of the American Society of Clinical Oncology 2011; 29: 228-234.
5 Cortez M A, Bueso-Ramos C, Ferdin J, Lopez-Berestein G, Sood A K, Calin G A. MicroRNAs in body fluids—the mix of hormones and biomarkers. Nature reviews Clinical oncology 2011; 8: 467-477.
6 Di Meo A, Bartlett J, Cheng Y, Pasic M D, Yousef G M. Liquid biopsy: a step forward towards precision medicine in urologic malignancies. Molecular cancer 2017; 16: 80.
7 Draisma G, Etzioni R, Tsodikov A, Mariotto A, Wever E, Gulati R et al. Lead time and overdiagnosis in prostate-specific antigen screening: importance of methods and context. Journal of the National Cancer Institute 2009; 101: 374-383.
8 Epstein J I, Zelefsky M J, Sjoberg D D, Nelson J B, Egevad L, Magi-Galluzzi C et al. A Contemporary Prostate Cancer Grading System: A Validated Alternative to the Gleason Score. European urology 2016; 69: 428-435.
9 Fendler A, Jung M, Stephan C, Honey R J, Stewart R J, Pace K T et al. miRNAs can predict prostate cancer biochemical relapse and are involved in tumor progression. International journal of oncology 2011; 39: 1183-1192.
10 Fendler A, Stephan C, Yousef G M, Kristiansen G, Jung K. The translational potential of microRNAs as biofluid markers of urological tumours. Nature reviews Urology 2016; 13: 734-752.
11 Fomicheva K A, Osip'yants Al, Knyazev E N, Samatov T R, Shkurnikov M Y. Detection of Potential Metastatic Prostate Cancer Circulating Biomarkers by Comparison of miRNA Profiles in DU145 Cells and Culture Medium. Bulletin of experimental biology and medicine 2017; 162: 792-796.
12 Garisto J D, Klotz L. Active Surveillance for Prostate Cancer: How to Do It Right. Oncology 2017; 31: 333-340, 345.
13 Klotz L, Zhang L, Lam A, Nam R, Mamedov A, Loblaw A. Clinical results of long-term follow-up of a large, active surveillance cohort with localized prostate cancer. Journal of clinical oncology: official journal of the American Society of Clinical Oncology 2010; 28: 126-131.
14 Lavi A, Cohen M. Prostate Cancer Early Detection Using Psa—Current Trends and Recent Updates. Harefuah 2017: 185-188.
15 Lichner Z, Fendler A, Saleh C, Nasser A N, Boles D, Al-Haddad S et al. MicroRNA signature helps distinguish early from late biochemical failure in prostate cancer. Clin Chem 2013; 59: 1595-1603.
16 Lichner Z, Ding Q, Samaan S, Saleh C, Nasser A, Al-Haddad S et al. miRNAs dysregulated in association with Gleason grade regulate extracellular matrix, cytoskeleton and androgen receptor pathways. J Pathol 2015; 237: 226-237.
17 Pierorazio P M, Walsh P C, Partin A W, Epstein J I. Prognostic Gleason Grade Grouping: data based on the modified Gleason scoring system. BJU international 2013; 111: 753-760.
18 Siegel R L, Miller K D, Jemal A. Cancer Statistics, 2017. C A: a cancer journal for clinicians 2017; 67: 7-30.
19 Tosoian J J, Trock B J, Landis P, Feng Z, Epstein J I, Partin A W et al. Active surveillance program for prostate cancer: an update of the Johns Hopkins experience. Journal of clinical oncology: official journal of the American Society of Clinical Oncology 2011; 29: 2185-2190.

1. A method for identifying low or high prostate cancer aggressiveness in a patient preoperative of prostatectomy with a known age and/or PSA level, comprising:
(a) (i) measuring exosomal miRNA levels in a biological sample to provide a biological sample exosomal miRNA profile comprising exosomal miRNA level of at least two, or at least three exosomal miRNA from miR-19a, miR-19b, miR-29a, miR-30c, miR-34a, miR-133a, miR-133b, miR-151-3p, miR-191, miR-331, miR-657, miR-664a and miR-875-3p, or
(ii) measuring exosomal miRNA levels in a biological sample to provide a biological sample exosomal miRNA profile comprising exosomal miRNA level of at least three exosomal miRNA from miR-19a, miR-19b, miR-26b, miR-29a, miR-29c, miR-30c, miR-34a, miR-99a, miR-133a, miR-133b, miR-151-3p, miR-191, miR-195, miR-331, miR-365, miR-374-5p, miR-378, miR-454, miR-590-5p, miR-657, miR-664a, and miR-875-3p; or
(iii) measuring the levels of at least two serum exosomal miRNAs selected from miR-29a, miR-30c, miR-151-3p, miR-191 and miR-664a, or
(iv) measuring the levels of at least two urine exosomal miRNAs selected from miR-19a, miR-195, miR-331, miR-374-5p and miR-590-5p; or
(v) measuring the levels of at least three serum exosomal miRNAs selected from miR-19a, miR-29a, miR-151-3p and miR-664a; or
(vi) measuring the levels of at least three urine exosomal miRNAs selected from miR-19a, miR-26b, miR-331, miR-374-5p and miR-590-5p; or
(vii) measuring the levels of at least two serum exosomal miRNAs selected from miR-29a, miR-133a, miR-151-3p and miR-657; or
(viii) measuring the levels of at least one serum exosomal miRNA selected from miR-29a, miR-133a, miR-151-3p and miR-657 and at least one urine exosomal miRNA selected from miR-19a, miR-26a, miR-331 and miR-590; and
(b) measuring the level of similarity of said biological sample exosomal miRNA profile to one or more control profiles, wherein (i) a high level of similarity of the sample exosomal miRNA profile to an active surveillance control profile; (ii) a low level of similarity to a prostatectomy control profile; and/or (iii) a higher level of similarity to an active surveillance control profile than to a prostatectomy control profile indicates low disease aggressiveness; or wherein (iv) a low level of similarity of the sample exosomal miRNA profile to an active surveillance control profile; (v) a high level of similarity to a prostatectomy control profile; and/or (vi) a lower level of similarity to an active surveillance control profile than to a prostatectomy control profile indicates high disease aggressiveness.

2. (canceled)

3. The method of claim 1 for identifying low or high prostate cancer aggressiveness in a patient preoperative of prostatectomy with a known age and/or PSA level, comprising the steps:
(a) providing at least one biological sample from a patient, wherein the biological sample is serum or urine;
(b) determining a biological sample profile comprising exosomal miRNA levels;
(c) measuring the level of similarity of said biological sample exosomal miRNA profile to one or more control profiles, wherein (i) a high level of similarity of the sample profile to an active surveillance control profile; (ii) a low level of similarity to a prostatectomy control profile; and/or (iii) a higher level of similarity to an active surveillance control profile than to a prostatectomy control profile indicates low disease aggressiveness; or wherein (iv) a low level of similarity of the sample profile to an active surveillance control profile; (v) a high level of similarity to a prostatectomy control profile; and/or (vi) a lower level of similarity to an active surveillance control profile than to a prostatectomy control profile indicates high disease aggressiveness;
(d) selecting said patient for active surveillance when said patient has low disease aggressiveness, or selecting said patient for prostatectomy when said patient has high disease aggressiveness; and
(e) repeating (a)-(d) in about one year when said patient is selected for active surveillance.

4. The method of claim 1, wherein the measuring of exosomal miRNA levels comprises using droplet digital PCR, digital molecular barcoding, or next-generation sequencing.

5. (canceled)

6. The method of claim 1, wherein a numerical score based on biological sample exosomal miRNA profile is calculated according to the following formula:
P=1/[1+exp.(−xβ)]=exp.(xβ)/[1+exp.(xβ)]
where xβ is standard linear form in multivariable logistic regression analysis,
wherein the numerical score is used in Receiver Operating Characteristic (ROC) analysis.

7. A method for predicting biochemical failure in a prostate cancer patient wherein
(A) the patient is preoperative of prostatectomy, the method comprising:
(a) (i) measuring exosomal miRNA levels in a biological sample to provide a biological sample exosomal miRNA profile comprising the levels of at least two serum exosomal miRNAs selected from miR-29a, miR-30c, miR-151-3p, miR-191 and miR-664a, or (ii) measuring exosomal miRNA levels in a biological sample to provide a biological sample exosomal miRNA profile comprising the levels of at least two urine exosomal miRNAs selected from miR-19a, miR-195, miR-331, miR-374-5p and miR-590-5p; and
(b) measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure; or
(B) the patient is preoperative of prostatectomy and has prostate cancer classified as Gleason Grade Group 1, 2, 3, or 4, the method comprising:
(a) (i) measuring exosomal miRNA levels in a biological sample to provide a biological sample exosomal miRNA profile comprising the levels of at least three serum exosomal miRNAs selected from miR-19a, miR-29a, miR-151-3p and miR-664a; or (ii) measuring exosomal miRNA levels in a biological sample to provide a biological sample exosomal miRNA profile comprising the levels of at least three urine exosomal miRNAs selected from miR-19a, miR-26b, miR-331, miR-374-5p and miR-590-5p; and
(b) measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure; or
(C) the patient is preoperative of prostatectomy and has prostate cancer classified as Gleason Grade Group 1, the method comprising:
(a) measuring exosomal miRNA levels in a biological sample to provide a biological sample exosomal miRNA profile comprising the level of (i) at least one of serum exosomal miRNAs miR-29a, miR-664a and miR-151-3p; (ii) serum exosomal miRNA miR-29a; (iii) serum exosomal miRNAs miR-657 and miR-151-3p, or (iv) serum exosomal miRNAs miR-133a and miR-151-3p; and
(b) measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure; or
(D) the patient is preoperative of prostatectomy and has prostate cancer classified as Gleason Grade Group 2, the method comprising:
(a) (i) measuring exosomal miRNA levels in a biological sample to provide a biological sample exosomal miRNA profile comprising the level of serum exosomal miRNAs miR-331; or (ii) measuring exosomal miRNA levels in a biological sample to provide a biological sample exosomal miRNA profile comprising the level of urinary exosomal miRNA miR-590-5p normalized by miR-29a; and
(b) measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure; or
(E) the patient is preoperative of prostatectomy and has prostate cancer classified as Gleason Grade Group 3, the method comprising:
(a) (i) measuring exosomal miRNA levels in a biological sample to provide a biological sample exosomal miRNA profile comprising the level of at least one of serum exosomal miRNAs miR-29a, miR-664a, and miR-151-3p; or (ii) measuring exosomal miRNA levels in a biological sample to provide a biological sample exosomal miRNA profile comprising the level of at least one of urinary exosomal miRNAs miR-590-5p, miR-195, miR-374-5p and miR-26b normalized by miR-29a; and
(b) measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure; or
(F) the patient is preoperative of prostatectomy with a known age and/or PSA level, the method comprising:
(a) (i) measuring exosomal miRNA levels in a biological sample to provide a biological sample exosomal miRNA profile comprising the levels of at least two serum exosomal miRNAs selected from miR-29a, miR-133a, miR-151-3p and miR-657; (ii) measuring exosomal miRNA levels in a biological sample to provide a biological sample exosomal miRNA profile comprising the levels of at least one serum exosomal miRNA selected from miR-29a, miR-133a, miR-151-3p and miR-657 and at least one urine exosomal miRNA selected from miR-19a, miR-26a, miR-331 and miR-590; (iii) measuring exosomal miRNA levels in a biological sample to provide a biological sample exosomal miRNA profile comprising the levels of at least three serum exosomal miRNA selected from miR-19a, miR-19b, miR-26b, miR-29a, miR-29c, miR-30c, miR-34a, miR-99a, miR-133a, miR-133b, miR-151-3p, miR-191, miR-195, miR-331, miR-365, miR-374-5p, miR-378, miR-454, miR-590-5p, miR-657, miR-664a, and miR-875-3p; or (iv) measuring exosomal miRNA levels in a biological sample to provide a biological sample exosomal miRNA profile comprising the levels of at least three urine exosomal miRNA selected from miR-19a, miR-19b, miR-26b, miR-29a, miR-29c, miR-30c, miR-34a, miR-99a, miR-133a, miR-133b, miR-151-3p, miR-191, miR-195, miR-331, miR-365, miR-374-5p, miR-378, miR-454, miR-590-5p, miR-657, miR-664a, and miR-875-3p; and
(b) measuring the level of similarity of said biological sample exosomal miRNA profile to one or more control profiles, wherein (i) a high level of similarity of the sample exosomal miRNA profile to an active surveillance control profile; (ii) a low level of similarity to a biochemical failure control profile; and/or (iii) a higher level of similarity to an active surveillance control profile than to a biochemical failure control profile predicts indolent disease; or wherein (iv) a low level of similarity of the sample exosomal miRNA profile to an active surveillance control profile; (v) a high level of similarity to a biochemical failure control profile; and/or (vi) a lower level of similarity to an active surveillance control profile than to a biochemical failure control profile predicts biochemical failure.

8. The method of claim 7, wherein predicting biochemical failure further comprises providing at least one biological sample from a patient, wherein the biological sample is serum or urine, and repeating steps (a) and (b) in about one year when said patient has indolent disease.

9. The method of claim 7, wherein the measuring of exosomal miRNA levels comprises using droplet digital PCR, digital molecular barcoding, or next-generation sequencing.

10. The method claim 7, wherein miRNA is measured as miRNA copies/mL sample.

11-30. (canceled)

31. A method for treating prostate cancer in a patient preoperative of prostatectomy with known age and/or PSA level, comprising:
(A) diagnosing said patient as a candidate for prostatectomy, comprising
(a) providing at least one biological sample from a patient, wherein the biological sample is serum or urine;
(b) (i) measuring said biological sample exosomal miRNA levels comprising exosomal miRNA level of at least one, at least two, or at least three exosomal miRNA from miR-19a, miR-19b, miR-29a, miR-30c, miR-34a, miR-133a, miR-133b, miR-151-3p, miR-191, miR-331, miR-657, miR-664a and miR-875-3p; or (ii) measuring said biological sample exosomal miRNA levels comprising exosomal miRNA level of at least three exosomal miRNA from miR-19a, miR-19b, miR-26b, miR-29a, miR-29c, miR-30c, miR-34a, miR-99a, miR-133a, miR-133b, miR-151-3p, miR-191, miR-195, miR-331, miR-365, miR-374-5p, miR-378, miR-454, miR-590-5p, miR-657, miR-664a, and miR-875-3p;
(c) determining said biological sample exosomal miRNA profile comprising miRNA levels of (b); and
(d) measuring the level of similarity of said biological sample exosomal miRNA profile to one or more control profiles, wherein (i) a low level of similarity of the sample profile to an active surveillance control profile; (ii) a high level of similarity to a prostatectomy control profile; and/or (iii) a lower level of similarity to an active surveillance control profile than to a prostatectomy control profile indicates high disease aggressiveness; or wherein (iv) a high level of similarity of the sample exosomal miRNA profile to an active surveillance control profile; (v) a low level of similarity to a prostatectomy control profile; and/or (vi) a higher level of similarity to an active surveillance control profile than to a prostatectomy control profile indicates low disease aggressiveness; and
(B) (i) removing the prostate when said patient has high disease aggressiveness, or
(ii) monitoring prostate cancer progression annually when said patient has low disease aggressiveness.

32. The method of claim 31, wherein the biological sample exosomal miRNA profile and the one or more control profiles comprise:
(a) the levels of at least two serum exosomal miRNAs selected from miR-29a, miR-30c, miR-151-3p, miR-191 and miR-664a, or at least two urine exosomal miRNAs selected from miR-19a, miR-195, miR-331, miR-374-5p and miR-590-5p;
(b) the levels of at least three serum exosomal miRNAs selected from miR-19a, miR-29a, miR-151-3p and miR-664a; or the levels of at least three urine exosomal miRNAs selected from miR-19a, miR-26b, miR-331, miR-374-5p and miR-590-5p; and/or
(c) the levels of at least two serum exosomal miRNAs selected from miR-29a, miR-133a, miR-151-3p and miR-657; or the levels of at least one serum exosomal miRNA selected from miR-29a, miR-133a, miR-151-3p and miR-657 and at least one urine exosomal miRNA selected from miR-19a, miR-26a, miR-331 and miR-590.

33. The method of claim 31, wherein the measuring of exosomal miRNA levels comprises using droplet digital PCR, digital molecular barcoding, or next-generation sequencing.

34. The method of claim 31, wherein miRNA is measured as miRNA copies/mL sample.

35. The method of claim 31, wherein a numerical score based on biological sample exosomal miRNA profile is calculated according to the following formula:
P=1/[1+exp.(−xβ)]=exp.(xβ)/[1+exp.(xβ)]
where xβ is standard linear form in multivariable logistic regression analysis,
wherein the numerical score is used in Receiver Operating Characteristic (ROC) analysis.

36-40. (canceled)

41. A kit for analyzing serum or urine sample to monitor prostate cancer progression in a patient comprising:
a probe that detects the presence of exosomal miRNA; and
instructions for use,
wherein the patient is preoperative of prostatectomy with a known age and/or PSA level,
wherein the probe is a set of exosomal miRNA-specific primers, and
wherein the set of exosomal miRNA-specific primers comprises primers targeting miR-19a, miR-19b, miR-26b, miR-29a, miR-29c, miR-30c, miR-34a, miR-99a, miR-133a, miR-133b, miR-151-3p, miR-191, miR-195, miR-331, miR-365, miR-374-5p, miR-378, miR-454, miR-590-5p, miR-657, miR-664a or miR-875-3p; or
at least two of miR-29a, miR-30c, miR-151-3p, miR-191 and miR-664a, or at least two of miR-19a, miR-195, miR-331, miR-374-5p and miR-590-5p; at least three of miR-19a, miR-29a, miR-151-3p and miR-664a, or
at least three of miR-19a, miR-26b, miR-331, miR-374-5p and miR-590-5p; at least two of miR-29a, miR-133a, miR-151-3p and miR-657; or
at least one of miR-19a, miR-133a, miR-151-3p and miR-657 and at least one of miR-19a, miR-26a, miR-331 and miR-590.

42-44. (canceled)

45. The kit of claim 41, wherein the set of exosomal miRNA-specific primers comprises primers targeting
at least two of miR-29a, miR-30c, miR-151-3p, miR-191 and miR-664a, or
at least two of miR-19a, miR-195, miR-331, miR-374-5p and miR-590-5p; or
at least three of miR-19a, miR-29a, miR-151-3p and miR-664a, or
at least three of miR-19a, miR-26b, miR-331, miR-374-5p and miR-590-5p; or
at least two of miR-29a, miR-133a, miR-151-3p and miR-657; or
at least one of miR-19a, miR-133a, miR-151-3p and miR-657 and at least one of miR-19a, miR-26a, miR-331 and miR-590.

46-47. (canceled)

48. The method of claim 1, comprising:
(A) (i) measuring exosomal miRNA levels in a biological sample to provide a biological sample exosomal miRNA profile comprising at least one, at least two, or at least three serum exosomal miRNA selected from miR-29a, miR-34a, miR-331, miR-664a, and miR-875-3p, and/or
(ii) measuring exosomal miRNA levels in a biological sample to provide a biological sample exosomal miRNA profile comprising at least one, at least two, or at least three urine exosomal miRNA selected from miR-26b, miR-29c, miR-99a, miR-195, miR-331, miR-374-5p, miR-454, and miR-590-5p; or
(B) (i) measuring exosomal miRNA levels in a biological sample to provide a biological sample exosomal miRNA profile comprising at least one, at least two, or at least three serum exosomal miRNA selected from miR-19b, miR-34a, miR-664a, and miR-875-3p, and/or
(ii) measuring exosomal miRNA levels in a biological sample to provide a biological sample exosomal miRNA profile comprising at least one, at least two, or at least three urine exosomal miRNA selected from miR-34a, miR-99a, miR-133b, miR-331, miR-374-5p, and miR-454.

49. The method of claim 7 part (F) comprises:
(A) (i) measuring exosomal miRNA levels in a biological sample to provide a biological sample exosomal miRNA profile comprising at least one, at least two, or at least three serum exosomal miRNA selected from miR-29a, miR-34a, miR-331, miR-664a, and miR-875-3p, and/or
(ii) measuring exosomal miRNA levels in a biological sample to provide a biological sample exosomal miRNA profile comprising at least one, at least two, or at least three urine exosomal miRNA selected from miR-26b, miR-29c, miR-99a, miR-195, miR-331, miR-374-5p, miR-454, and miR-590-5p; or
(B)(i) measuring exosomal miRNA levels in a biological sample to provide a biological sample exosomal miRNA profile comprising at least one, at least two, or at least three serum exosomal miRNA selected from miR-19b, miR-34a, miR-664a, and miR-875-3p, and/or
(ii) measuring exosomal miRNA levels in a biological sample to provide a biological sample exosomal miRNA profile comprising at least one, at least two, or at least three urine exosomal miRNA selected from miR-34a, miR-99a, miR-133b, miR-331, miR-374-5p, and miR-454.

50. The method of claim 31, comprising:
(A) (i) measuring exosomal miRNA levels in a biological sample to provide a biological sample exosomal miRNA profile comprising at least one, at least two, or at least three serum exosomal miRNA selected from miR-29a, miR-34a, miR-331, miR-664a, and miR-875-3p, and/or
(ii) measuring exosomal miRNA levels in a biological sample to provide a biological sample exosomal miRNA profile comprising at least one, at least two, or at least three urine exosomal miRNA selected from miR-26b, miR-29c, miR-99a, miR-195, miR-331, miR-374-5p, miR-454, and miR-590-5p; or
(B) (i) measuring exosomal miRNA levels in a biological sample to provide a biological sample exosomal miRNA profile comprising at least one, at least two, or at least three serum exosomal miRNA selected from miR-19b, miR-34a, miR-664a, and miR-875-3p, and/or
(ii) measuring exosomal miRNA levels in a biological sample to provide a biological sample exosomal miRNA profile comprising at least one, at least two, or at least three urine exosomal miRNA selected from miR-34a, miR-99a, miR-133b, miR-331, miR-374-5p, and miR-454.

51. The method of claim 7, wherein the method further comprises:
(c) identifying Gleason Grade or tumor volume of prostatectomy of said patient based on exosomal miRNA levels.

52. The method of claim 31, wherein the patient was previously misassigned as having aggressive disease or indolent disease.

53. The method of claim 31, further comprising selecting said patient for prostatectomy when said patient has high disease aggressiveness indicated by monitoring prostate cancer progression annually.
